(19) United States
(12) Patent Application Publication
(10) Pub. No.: US 2023/0250149 A1
(43) Pub. Date: Aug. 10, 2023

(54) ANTIGEN-SPECIFIC T CELL RECEPTORS AND CHIMERIC ANTIGEN RECEPTORS, AND METHODS OF USE IN IMMUNE SIGNALING MODULATION FOR CANCER IMMUNOTHERAPY

(71) Applicant: THE METHODIST HOSPITAL, Houston, TX (US)

(72) Inventors: Rongfu WANG, Temple City, CA (US); Yicheng WANG, Temple City, CA (US); Bingnan YIN, Alhambra, CA (US); Chen QIAN, Sherman Oaks, CA (US); Xin LIU, Alhambra, CA (US)

(21) Appl. No.: 18/002,969

(22) PCT Filed: Jun. 25, 2021
(86) PCT No.: PCT/US2021/039262

§ 371 Date: Dec. 22, 2022

Related U.S. Application Data
(60) Provisional application No. 63/044,150, filed on Jun. 25, 2020.

(51) Int. Cl. C07K 14/725; C07K 14/74; C12N 15/63; A61P 35/00

(52) U.S. Cl. CPC C07K 14/7051; C07K 14/70539; C12N 15/63; A61P 35/00; C07K 2317/24

(57) ABSTRACT

The present invention relates to T cell receptors (TCR) against cancer/testis antigens NY-ESO-1 and CT83 presented by multiple HLA molecules. The preferred TCRs of the invention deriving from human T cells demonstrates high affinity and antigen specificity in vitro and in vivo. The present invention also relates to the modulation of TCR-T CAR-T cell signaling and functional persistence in cancer immunotherapy.

### FIELD OF THE INVENTION

This invention relates to methods for the identification and functional validation of T cell receptors (TCRs) from tumor antigen specific T cells; modulation of TCR-T and chimeric antigen receptor (CAR)-T cells to increase and prolong T cell persistence and reduce T cell exhaustion by direct manipulation of TCR or CAR signaling domains and knockdown/knockout of negative signaling molecules; and methods of using the TCRs, TCR-T and CAR-T cells in the treatment of cancer. This invention also relates to the identified polypeptides comprising one or a plurality of alpha and beta chains of T-cell receptors (“TCRs”) specific to cancer antigens, e.g., NY-ESO-1 (“ESO-1 TCR″), CT83 (“CT83-TCR”), and viral antigens, e.g., human cytomegalovirus (HCMV) PP65 and (HCMV) IE1 TCRs, nucleic acids and recombinant vectors encoding the polypeptides, and cells comprising the nucleic acids or recombinant vectors.

### BACKGROUND OF THE INVENTION

The host immune system comprises innate and adaptive immunity to recognize and eliminate exogenous or endogenous antigens derived from pathogens or abnormal tissues, including cancer cells. Schreiber, R. D., Old, L. J. & Smyth, M. J., Cancer immunoediting: integrating immunity's roles in cancer suppression and promotion. Science 331, 1565-1570, doi:10.1126/science.1203486 (2011); Vesely, M. D., Kershaw, M. H., Schreiber, R. D. & Smyth, M. J., Natural innate and adaptive immunity to cancer. Annual review of immunology 29, 235-271, doi:10.1146/annurev-immunol-031210-101324 (2011). Various types of immune cells contribute to cancer cell recognition, suppression and rejection. Although tumor-reactive T lymphocytes (T cells) were demonstrated to play a direct role in tumor rejection, the clinical response to early cancer immunotherapies was limited to due to several factors, especially immune suppression. More recently, the identification of immune checkpoints has led to development of targeted immunotherapies. The depletion or suppression of immune suppression checkpoints, e.g, programmed cell death-1 protein (PD1) and its ligand PD-L1, cytotoxic T lymphocyte antigen-4 (CTLA-4) and other checkpoint inhibitors, has profoundly enhanced antitumor immunity and shown impressive and durable clinical responses in many types of cancer patients. Callahan, M. K., et al., Anti-CTLA-4 antibody therapy: immune monitoring during clinical development of a novel immunotherapy. Seminars in oncology 37, 473-484, doi:10.1053/j.seminonco1.2010.09.001 (2010); Chambers, C. A., Kuhns, M. S., Egen, J. G. & Allison, J. P., CTLA-4-mediated inhibition in regulation of T cell responses: mechanisms and manipulation in tumor immunotherapy. Annual review of immunology 19, 565-594, doi:10.1146/annurev.immunol.19.1.565 (2001); Zhu, Y., Yao, S. & Chen, L. Cell surface signaling molecules in the control of immune responses: a tide model, Immunity 34, 466-478, doi:10.1016/j.immuni.2011.04.008 (2011); Wang, H. Y. & Wang, R. F. Regulatory T cells and cancer. Current opinion in immunology 19, 217-223, doi:10.1016/j.coi.2007.02.004 (2007); Joyce, J. A. & Fearon, D. T. T cell exclusion, immune privilege, and the tumor microenvironment. Science 348, 74-80, doi:10.1126/science.aaa6204 (2015). Benefitting from the breakthrough in cancer immunotherapy, T cell-based immunotherapy has recently been successfully applied to treat human cancers which can include or exclude melanoma, renal cell carcinoma and lymphoma with varying degrees of tumor regression.

CD8+ and CD4+ T cells are the major components of T cell-based antitumor immunity. CD8+ T cells, also known as cytotoxic T lymphocytes (CTL), are capable of specifically recognizing a complex of an epitope bound to a class I molecule of a major histocompatibility complex (MHC—termed human leukocyte antigen, HLA in humans) via a T cell receptor (TCR) and killing a cell when the complex is presented on the cell surface. CD4+ T cells, mainly referred to as T helper cells (Th cells), are another type of T cell that plays an important role in the immune system. CD4+ T cells are capable of specifically recognizing a complex of an epitope bound to a class II molecule of an MHC via a TCR and releasing cytokines and regulating the immune system. CD4+ T cells are also essential in the activation of CD8+ T cells, B lymphocytes and other immune cells which can include or exclude macrophages.

To initiate a tumor-specific T cell response, tumor antigens are processed and degraded into peptides comprising 9-13 amino acids (referred to as an epitope) in the tumor cells or other antigen-processing cells (APC) via the proteasome pathway (for major histocompatibility (MHC) class I molecule binding) or endosome/lysosome pathway (for MHC class II molecule binding). The final products of such antigen processing bind to a certain type of MHC class I or II molecule of the APCs and are transported onto the cell surface, which may activate CD8+ or CD4+ T cells when the epitope-HLA complex specifically bind to a TCR on the T cell surface. CD8+ T cell cytotoxic activity can kill tumor cells directly. However, other work demonstrates that CD4+ T cells also play a role in antitumor immunity. Wang, R. F. & Rosenberg, S. A. Human tumor antigens for cancer vaccine development. Immunological reviews 170, 85-100 (1999); Wang, R. F. The role of MHC class II-restricted tumor antigens and CD4+ T cells in antitumor immunity. Trends in immunology 22, 269-276 (2001). Moreover, a subset of CD4+ T cells (CD4 CTL) possess cytotoxic activity and are directly involved in tumor cell killing in a HLA class II restricted fashion. Takeuchi, A. & Saito, T. CD4 CTL, a Cytotoxic Subset of CD4(+) T Cells, Their Differentiation and Function. Frontiers in immunology 8, 194, doi:10.3389/fimmu.2017.00194 (2017); Wang, R. F. & Wang, H. Y. Immune targets and neoantigens for cancer immunotherapy and precision medicine. Cell research 27, 11-37, doi:10.1038/cr.2016.155 (2017).

Chimeric antigen receptor (CAR) engineered T cells have produced durable clinical benefits for blood cancers, including leukemia and lymphoma. CD19-CAR-T products have been approved by U.S. Food Drug Administration (FDA) for the treatment of lymphoma and leukemia. June, C. H. & Sadelain, M. Chimeric Antigen Receptor Therapy. N Engl J Med 379, 64-73, doi:10.1056/NEJMra1706169 (2018). However, CAR-T cell therapy has not worked well for treating solid cancers. Furthermore, approximately 30-50% of CD19-CAR-T treated cancer patients, who achieve remission, will have disease relapse within 12 months of treatment. Shah, N. N. & Fry, T. J. Mechanisms of resistance to CAR T cell therapy. Nat Rev Clin Oncol 16, 372-385, doi:10.1038/s41571-019-0184-6 (2019); Park, J. H. et al. Long-Term Follow-up of CD19 CAR Therapy in Acute Lymphoblastic Leukemia. N Engl J Med 378, 449-459, doi:10.1056/NEJMoa1709919 (2018); Maude, S. L. et al. Tisagenlecleucel in Children and Young Adults with B-Cell Lymphoblastic Leukemia. N Engl J Med 378, 439-448, doi:10.1056/NEJMoa1709866 (2018).

### SUMMARY

In one aspect, this disclosure relates to methods for identifying and functionally validating TCRs from cancer antigen specific T cells. For example, in some aspects, this disclosure relates to methods for identifying and functionally validating TCRs from NY-ESO-1-specific, CT83-specific, human cytomegalovirus (HCMV)-pp65-specific and/or HCMV-IE-1-specific T cells.

In some aspects, this disclosure features methods of detecting and cloning TCRs from cancer antigen specific T-cells (as non-limiting examples, the cancer antigen specific T-cells can include or exclude any of NY-ESO-1-specific, CT83-specific, HCMV-pp65-specific and/or HCMV-IE-1-specific T cells) in a human subject, the method comprising: a) stimulating naïve T cells with cancer antigens, e.g., Class I or Class II HLA-restricted epitope complexes (as non-limiting examples, including or excluding any of Class II HLA-DP4-restricted NY-ESO-1 epitope complexes, Class I HLA-A2-restricted CT83 epitope complexes, Class I HLA-A2-restricted HCMV-pp65 epitope complexes, and/or Class I HLA-A2-restricted HCMV-IE-1 epitope complexes) in vitro; b) detecting T cell populations specific for cancer antigen epitopes (as a non-limiting example, including or excluding any of NY-ESO-1, CT83, HCMV-pp65 and/or HCMV-IE-1 epitopes) in vitro and in vivo; c) sorting cancer antigen epitope specific CD4+ or CD8+ T-cell populations (as non-limiting examples, including or excluding any of NY-ESO-1, CT83, HCMV-pp65, and/or HCMV-IE-1 epitope-specific CD4+ or CD8+ T-cell populations); d) isolating single T cells from the sorted T cells; e) obtaining T cell V(D)J sequences from the T cells by single-cell next-generation sequencing; f) synthesizing primers for TCR cloning based on the sequences; g) amplifying TCR variable regions of alpha and beta chains from pooled T cells stimulated by the cancer antigen(s) (as non-limiting examples, including or excluding any of NY-ESO-1 epitopes, CT83 epitopes, HCMV-pp65 epitopes and/or HCMV-IE-1 epitopes; and h) constructing amplified TCR variable regions of alpha and beta chains into vectors to form a complete TCR construct. In some aspects, the method further comprises the steps of; j) transducing cloned TCRs into naïve CD4+ or CD8+ T cells; i) measuring transduced T cell activity k) and screening transduced T cells for binding to, recognition of and/or activation by multiple targets in vitro and in vivo (for example, one or more peptides, one or more cells or cell lines, transfected cell lines, and/or one or more tumor cell lines). In one aspect, the TCRs from the cancer antigen specific T-cells identified by the methods in the preceding aspects or other aspects and embodiments described herein can bind and/or recognize any cancer antigen described herein, including any cancer antigen or fragment or epitope thereof, as described in any aspect or embodiment described herein, including any cancer antigen described in the discussion of “cancer antigen” and “tumor antigen.”

In one aspect, also disclosed herein are methods of identifying epitope-specific T cells and TCRs of any preceding aspect or any aspect or embodiment disclosed herein, wherein the one or more cell or cell lines (which can include or exclude, for example HEK293 cells, HEK293T cells, Cos-7 cells, 586-mel cells, 624-mel cells, MDA-MB-231 cells, MDA-MB-436 cells, E0771 cells, HTB-21 cells). In one aspect, the one or more tumor cell lines can include or exclude any cell line selected from the group consisting of B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers, small cell lung cancer, non-small cell lung cancer, neuroblastoma, glioblastoma, ovarian cancer, pancreatic cancer, prostate cancer, skin cancers, melanoma, basal cell carcinoma, squamous cell carcinoma, liver cancer, squamous cell carcinomas of the mouth, throat, larynx, and lung, cervical cancer, cervical carcinoma, breast cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, AIDS-related lymphomas, or AIDS-related sarcomas, and may be optionally selected from: HEK293 cells, HEK293T cells, Cos-7 cells, 586-mel cells, 624-mel cells, MDA-MB-231 cells, MDA-MB-436 cells, E0771 cells, HTB-21 cells.

In one aspect, also disclosed herein are methods of identifying epitope-specific T cells and TCRs of any preceding aspect or any aspect or embodiment disclosed herein, wherein the the one or more cell or cell lines are engineered to express an MHC Class I or Class II molecule.

Also disclosed herein are methods of identifying epitope-specific T cells and TCRs of any preceding aspect or any aspect or embodiment disclosed herein, wherein the measured T cell activity (which can include or exclude, for example, release of cytokines including, but not limited to IFN-α, TGF-β, lymphotoxin-α, IL-2, IL-4, IL-10, IL-17, or IL-25) is measured by any immunodetection method disclosed herein. In some embodiments, the T cell activity may be measured, for example, without limitation, by ELISA, chemiluminescence, by ELISPOT, Intracellular cytokine staining, or Chromium Release, or any other immunodetection disclosed herein.

In one aspect, also disclosed herein are methods of identifying epitope-specific T cells and TCRs of any preceding aspect or any aspect or embodiment disclosed herein, wherein the cancer is selected from and can include or exclude any of the group consisting of B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers, small cell lung cancer, non-small cell lung cancer, neuroblastoma, glioblastoma, ovarian cancer, pancreatic cancer, prostate cancer, skin cancers, melanoma, basal cell carcinoma, squamous cell carcinoma, liver cancer, squamous cell carcinomas of the mouth, throat, larynx, and lung, cervical cancer, cervical carcinoma, breast cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, AIDS-related lymphomas, or AIDS-related sarcomas.

In one aspect, also disclosed herein are cancer epitopes which can include or exclude epitopes from any cancer antigen or tumor antigen recognized or bound by cancer antigen specific T-cells and TCRs identified by the methods of detecting or identifying cancer antigen/epitope-specific T cells and TCRs of any preceding aspect or any aspect or embodiment disclosed herein.

In one aspects the cancer epitopes may include or exclude epitopes of NY-ESO-1 identified by the methods of detecting or identifying epitope-specific T cells and TCRs of any preceding aspect or any aspect or embodiment disclosed herein. For example, disclosed herein are a polypeptide comprising the amino acid sequence SLLMWITQCFLPVF (Seq ID NO: 1), and variants thereof, as disclosed herein.

In one aspect, also disclosed herein are cancer epitopes which can include or exclude epitopes of CT83 identified by the methods of detecting or identifying epitope-specific T cells and TCRs of any preceding aspect or any aspect or embodiment disclosed herein. In some aspects the epitopes may consist essentially of an identified epitope. The basic and essential property of an epitope is that it is a portion of the target protein that can be recognized or bound by a TCR in the context of either Class I or Class II MHC. For example, disclosed herein are a polypeptide comprising the amino acid sequence KLVELEHTL (Seq ID NO: 2), and variants thereof, as disclosed herein.

In one aspects the cancer epitopes may include or exclude epitopes of HCMV-pp65 identified by the methods of detecting or identifying epitope-specific T cells and TCRs of any preceding aspect or any aspect or embodiment disclosed herein. For example, disclosed herein are a polypeptide comprising the amino acid sequence NLVPMVATV (SEQ ID NO:26) and variants thereof, as disclosed herein.

In one aspects the cancer epitopes may include or exclude epitopes of HCMV-IE-1 identified by the methods of detecting or identifying epitope-specific T cells and TCRs of any preceding aspect or any aspect or embodiment disclosed herein. For example, disclosed herein are a polypeptide comprising the amino acid sequence VLEETSVML (SEQ ID NO:31) and variants thereof, as disclosed herein.

In one aspect, this disclosure relates to compositions comprising one or a plurality of alpha chains and/or beta chains of T-cell receptors (“TCRs”) specific to cancer antigens, identified or detected by the methods of identifying or detecting epitope-specific T cells and TCRs of any preceding aspect or any aspect or embodiment disclosed herein. In some aspects, the alpha chains and/or beta chains of the TCRs recognize and/or bind to cancer antigens, for example, without limitation, NY-ESO-1 (“ESO-1 TCR”) and/or CT83 (“CT83-TCR”). In some aspects, the the alpha chains and/or beta chains of the TCRs recognize and/or bind to cancer antigens of viral origin, for example, viral antigens expressed on human or mammalian cancer cells. In some aspects the the alpha chains and/or beta chains of the TCRs recognize and/or bind to cancer antigens, for example, without limitation, proteins from human cytomegalovirus (HCMV). In some aspects the the alpha chains and/or beta chains of the TCRs recognize and/or bind to cancer antigens are HCMV pp65 and HCMV IE-1 proteins or fragments or epitopes thereof. In some aspects the alpha chains and/or beta chains of the TCRs recognize and/or bind to HCMV pp65 (amino acids 495-503) and/or HCMV IE-1 (amino acids 316-324). In some aspects the the alpha chains and/or beta chains of the TCRs recognize and/or bind to cancer antigens which can include or exclude any of the cancer antigens disclosed above.

In some aspects the present disclosure relates to compositions comprising, for example, one or a plurality of alpha chain or region and/or one or a plurality of beta chain or region of T-cell receptors specific to cancer antigens. In some aspects, the present disclosure relates to compositions comprising, for example, one or a plurality of alpha chain/region or beta chain/region of T-cell receptors specific to a cancer antigen which can include or exclude any of NY-ESO-1 (ESO-1 TCR), CT83 (CT83-TCR), HCMV-pp65 (pp65-TCR) and/or HCMV-IE-1 (IEI-TCR) or any combination thereof. In some aspects, the compositions comprise, for example, at least one polypeptide comprising the alpha chain or region of T-cell receptors specific to NY-ESO-1 (ESO-1 TCR), CT83 (CT83-TCR), pp65 (pp65-TCR) or IE-1 (IE-1-TCR) and at least one polypeptide comprising the beta chain of a T-cell receptor specific to NY-ESO-1 (ESO-1 TCR) or CT83 (CT83-TCR) HCMV-pp65 (pp65-TCR) or HCMV-IE-1 (IE-1-TCR). In some aspects, the compositions comprise, for example, one polypeptide comprising the alpha chain or region of a T-cell receptos specific to NY-ESO-1 (ESO-1 TCR), CT83 (CT83-TCR), pp65 (pp65-TCR) or IE-1 (IE-1-TCR) and one polypeptide comprising the beta chain or region of a T-cell receptor specific to NY-ESO-1 (ESO-1 TCR), CT83 (CT83-TCR), HCMV-pp65 (pp65-TCR) or HCMV-IE-1 (IE-1-TCR), respectively.

In one aspect, also disclosed herein are alpha variable region of cancer antigen specific TCRs detected or identified by the methods detecting or identifying epitope-specific T cells and TCRs of any preceding aspect or any aspect or embodiment disclosed herein. In one aspect, the alpha variable region can include or exclude the alpha variable region of DP4-ESO-1 TCR, the alpha variable region of A2-CT83 TCR, the alpha variable region of A2-pp65 TCR, and/or the alpha region of A2-IE-1-TCR detected or identified by the methods detecting or identifying epitope-specific T cells and TCRs of any preceding aspect or any aspect or embodiment disclosed herein, and/or for use with any variable region sequence or epitope specific sequence identified herein. For example, disclosed herein are a polypeptide comprising the amino acid sequence METVLQVLLGILGFQAAWVSSQELEQSPQSLIVQEGKNLTINCTSSKTLYGLYWYKQ KYGEGLIFLMMLQKGGEEKSHEKITAKLDEKKQQSSLHITASQPSHAGIYLCGADIVD YGQNFVFGPGTRLSVLPY (Seq ID NO: 3) (alpha variable region of DP4-ESO-1 TCR). As another example, disclosed herein are a polypeptide comprising the amino acid sequence MKTFAGFSFLFLWLQLDCMSRGEDVEQSLFLSVREGDSSVINCTYTDSSSTYLYWYK QEPGAGLQLLTYIFSNMDMKQDQRLTVLLNKKDKHLSLRIADTQTGDS AIYFCAEKS GYSGAGSYQLTFGKGTKLSVIPN (SEQ ID NO: 5) (alpha variable region of A2-CT83 TCR). As another example, disclosed herein are a polypeptide comprising the amino acid sequence MEKNPLAAPLLILWFHLDCVSSILNVEQSPQSLHVQEGDSTNFTCSFPSSNFYALHWY RWETAKSPEALFVMTLNGDEKKKGRISATLNTKEGYSYLYIKGSQPEDSATYLCARN TGNQFYFGTGTSLTVIPN (SEQ ID NO:29) (alpha variable region of A2-pp65 TCR. As another example disclosed herein are a polypeptide comprising the amino acid sequence MLLITSMLVLWMQLSQVNGQQVMQIPQYQHVQEGEDFTTYCNSSTTLSNIQWYKQ RPGGHPVFLIQLVKSGEVKKQKRLTFQFGEAKKNSSLHITATQTTDVGTYFCAGHIY GGSQGNLIFGKGTKLSVKPN (SEQ ID NO:32) (alpha variable region of A2-IE-1-TCR). Also disclosed herein are fragments or variants of any polypeptide or polypeptide fragment in any preceding aspect or any embodiment disclosed herein which bind to the antigen with the same specificity as the reference (full length and unmodified) receptor. In some aspects, the variant comprises a conservative amino acid substitution as disclosed further herein. Substitutions to any of the alpha variable regions disclosed herein may include or exclude substitutions in one or more of the 6 CDRs of the TCRs.

In one aspect, also disclosed herein are beta variable region of cancer antigen specific TCRs identified by the methods identifying epitope-specific T cells and TCRs of any preceding aspect, and/or for use with any variable region sequence or epitope specific sequence identified herein. In one aspect, the beta variable region can include or exclude the beta variable region of DP4-ESO-1 TCR, the beta variable region of A2-CT83 TCR, the beta variable region of A2-pp65 TCR, and/or the beta region of A2-IE-1-TCR detected or identified by the methods detecting or identifying epitope-specific T cells and TCRs of any preceding aspect or any aspect or embodiment disclosed herein, and/or for use with any variable region sequence or epitope specific sequence identified herein. In one aspect, also disclosed herein are beta variable region of cancer specific TCRs DP4-ESO-1 TCR identified by the methods identifying epitope-specific T cells and TCRs of any preceding aspect, and/or for use with any variable region sequence or epitope specific sequence identified herein. For example, disclosed herein are a polypeptide comprising the amino acid sequence MLCSLLALLLGTFFGVRSQTIHQWPATLVQPVGSPLSLECTVEGTSNPNLYWYRQAA GRGLQLLFYSVGIGQISSEVPQNLSASRPQDRQFILSSKKLLLSDSGFYLCAWRRRGY EQYFGPGTRLTVTE (Seq ID NO: 4). In one aspect, also disclosed herein are beta variable region of A2-CT83 TCR detected or identified by the methods of detecting or identifying epitope-specific T cells and TCRs of any preceding aspect, and/or for use with any variable region sequence or epitope specific sequence identified herein. For example, disclosed herein are a polypeptide comprising the amino acid sequence MLSLLLLLLGLGSVFSAVISQKPSRDICQRGTSLTIQCQVDSQVTMMFWYRQQPGQS LTLIATANQGSEATYESGFVIDKFPISRPNLTFSTLTVSNMSPEDSSIYLCSVQDSEAFF GQGTRLTVVE (Seq ID NO: 6). In one aspect, also disclosed herein are beta variable region of A2-pp65 TCR detected or identified by the methods of detecting or identifying epitope-specific T cells and TCRs of any preceding aspect, and/or for use with any variable region sequence or epitope specific sequence identified herein. For example, disclosed herein are a polypeptide comprising the amino acid sequence MSIGLLCCAALSLLWAGPVNAGVTQTPKFQVLKTGQSMTLQCAQDMNHEYMSWY RQDPGMGLRLIHYSVGAGITDQGEVPNGYNVSRSTTEDFPLRLLSAAPSQTSVYFCAS SPITGTGDYGYTFGSGTRLTVVE (SEQ ID NO:30). In one aspect, also disclosed herein are beta variable region of A2-IE-1 TCR detected or identified by the methods of detecting or identifying epitope-specific T cells and TCRs of any preceding aspect, and/or for use with any variable region sequence or epitope specific sequence identified herein. For example, disclosed herein are a polypeptide comprising the amino acid sequence MGSRLLCWVLLCLLGAGPVKAGVTQTPRYLIKTRGQQVTLSCSPISGHRSVSWYQQ TPGQGLQFLFEYFSETQRNKGNFPGRFSGRQFSNSRSEMNVSTLELGDSALYLCASSH HQGPLETQYFGPGTRLLVLE (SEQ ID NO:33). Also disclosed herein are variants of any polypeptide or polypeptide fragment in any preceding aspect or any embodiment disclosed herein. In some aspects, the variant comprises a conservative amino acid substitution as disclosed further herein. Substitutions to any of the beta variable regions disclosed herein may include or exclude substitutions in one or more of the 6 CDRs of the TCRs.

Also disclosed herein are variants of any polypeptide or polypeptide fragment disclosed in any preceding aspect or any embodiment disclosed herein wherein the variant comprises a conservative amino acid substitution. Substitutions may include or exclude substitutions in one or more of the 6 CDRs of the TCRs.

Also disclosed herein are variants of any polypeptide or polypeptide fragment disclosed herein wherein the variant comprises a conservative amino acid substitution. Substitutions may include or exclude substitutions in one or more of the 6 CDRs of the TCRs.

In one aspect, this disclosure includes chimeric TCRs comprising a TCR variable region fused to a modified human constant region, or to a non-human constant region which can be modified or unmodified. In some aspects the chimeric TCR comprises a cancer antigen specific TCR variable region fused to a non-human, e.g., murine TCR constant region. In some aspects the TCR variable region comprises an alpha and a beta chain variable region fused to an alpha and a beta TCR constant region, respectively and can include any alpha and/or beta chain of any other aspect of this disclosure. In some aspects, fusion of the TCR variable region to a modified or non-human constant region reduces mispairing between the chimeric TCR and an endogenous TCR. In some aspects the chimeric TCR comprises a variable region that can include or exclude any one of a CT83 TCR variable region, a NY-ESO-1 TCR variable region, a pp65 TCR variable region and/or a IE-1 TCR variable region fused to a non-human, e.g., murine TCR constant region. Among other aspects, the chimeric TCR reduces mispairing between the chimeric TCR and the endogenous TCR of the transduced T-cell. For example, in some aspects, a chimeric CT83 TCR reduces mispairing between the chimeric CT83 TCR(MC) and endogenous TCR(HC). In other aspects, for example, a chimeric NY-ESO-1 TCR reduces mispairing between the chimeric NY-ESO-1 (MC) and endogenous TCR(HC). In other aspects, for example, a chimeric pp65 TCR reduces mispairing between the chimeric pp65 (MC) and endogenous TCR(HC). In other aspects, for example, a chimeric IE-1 TCR reduces mispairing between the chimeric IE-1 (MC) and endogenous TCR(HC).

In one aspect, this disclosure includes chimeric TCRs comprising a TCR variable region fused to a modified human constant region, or non-human constant region which can be unmodified or modified. In some aspects, the chimeric TCR comprises a cancer antigen specific TCR variable region fused to a non-human, e.g., murine TCR constant region. In some aspects the TCR variable region comprises an alpha and a beta chain variable region fused to an alpha and, a beta TCR constant region, respectively and can include any alpha and/or beta chain of any other aspect o this disclosure. In some aspects, the chimeric TCR comprises a TCR variable region that includes or excludes any of a CT83 TCR variable region, a NY-ESO-1 TCR variable region, a pp65 TCR variable region, or a IE-1 TCR fused to a non-human, e.g., murine TCR constant region. Among other aspects, the chimeric TCR reduces mispairing between the chimeric TCR and the endogenous TCR of the transduced T-cell. For example, in some aspects, a chimeric CT83 TCR reduces mispairing between the chimeric CT83 TCR(MC) and endogenous TCR(HC). In other aspects, for example, a chimeric NY-ESO-1 TCR reduces mispairing between the chimeric NY-ESO-1 (MC) and endogenous TCR(HC) or reduces mispairing. In other aspects, for example, a chimeric pp65 TCR reduces mispairing between the chimeric pp65 (MC) and endogenous TCR(HC) or reduces mispairing. In other aspects, for example, a chimeric IE-1 TCR reduces mispairing between the chimeric IE-1 (MC) and endogenous TCR(HC) or reduces mispairing.

Also featured are nucleic acids encoding any of the above-mentioned epitopes, receptor chains and/or polypeptides or any other polypeptide disclosed in any aspect or embodiment herein; recombinant nucleic acids containing the above-mentioned nucleic acids; vectors or constructs comprising the above-mentioned recombinant nucleic acids; and cells into which one or a plurality of the above-mentioned nucleic acids or vectors is transduced.

In one aspect, also disclosed herein are nucleic acids encoding a polypeptide comprising the epitope of any preceding aspect.

In one aspect, also disclosed herein are nucleic acids encoding a polypeptide TCR alpha and/or beta variable regions or chains of any preceding aspect or any embodiment disclosed herein. In one aspect the nucleic acids of this disclosure encode any one of SEQ ID NOs: 3-6, 10-11, 12-15, 20-25, 27-30, 32, or 33. In one aspect the nucleic acid has the sequence of any one of SEQ ID Nos: 41-50, which encode the TCR variable regions noted below:

| | Alpha variable | | Beta variable | |
|---|---|---|---|---|
| TCR | Amino acid | DNA | Amino acid | DNA |
| DP4-ESO-1 TCR | Seq ID NO: 3 | Seq ID NO: 41 | Seq ID NO: 4 | Seq ID NO: 42 |
| A2-CT83 TCR | Seq ID NO: 5 | Seq ID NO: 43 | Seq ID NO: 6 | Seq ID NO: 44 |
| HCMV pp65 TCR | Seq ID NO: 27, | Seq ID NO: 45, | Seq ID NO: 28, | Seq ID NO: 46, |
| | Seq ID NO: 29 | Seq ID NO: 47 | Seq ID NO: 30 | Seq ID NO: 48 |
| HCMV IE1 TCR | Seq ID NO: 32 | Seq ID NO: 49 | Seq ID NO: 33 | Seq ID NO: 50 |

In one aspect, the nucleic acid sequences may have one or more codon substitutions which do not alter the sequence of the encoded polypeptide.

In some aspects, this disclosure also features nucleic acids encoding any of the TCR regions or chains of this disclosure. In some aspects, the nucleic acids may further comprise a signaling component. In some aspects, the signaling component can include or exclude ZAP327 (SEQ ID NO:17) or ZAP 300 (SEQ ID NO:16) or another signaling component derived from a ZAP70 kinase domain. In some aspects the signaling component confers increased persistence and/or anti-tumor activity on cells engineered with these nucleic acids.

Also disclosed herein are compositions comprising a therapeutically effective amount of one or more of the TCR alpha or beta variable region of any preceding aspect or any aspect or embodiment disclosed herein. In some aspects, the compositions may also comprise any signaling component of any preceding aspect, and for example can include or exclude ZAP327 (SEQ ID NO:17) or ZAP 300 (SEQ ID NO:16) or another signaling component derived from a ZAP70 kinase domain.

Also disclosed herein are compositions comprising a therapeutically effective amount of one or more TCR T-cells; wherein the TCR T cell has been engineered to express any of the nucleic acids of any preceding aspect or any aspect or embodiment disclosed herein. In some aspects, the TCR T cell may be engineered to express, for example, a receptor (which can include or exclude, for example, a T cell receptor) that recognizes one or more of the cancer antigens or neoantigens of any preceding aspect or any aspect or embodiment disclosed herein. In some aspects, the TCR T-cells may express one or a plurality of the TCR alpha and/or one or a plurality of beta variable regions of any preceding aspect or any aspect or embodiment disclosed herein of this disclosure. In some aspects these TCR T-cells can also include or exclude ZAP327 (SEQ ID NO:17) or ZAP 300 (SEQ ID NO:16) or another signaling component derived from a ZAP70 kinase domain. In some aspects, the engineered TCR T cells comprising and/or expressing the signaling component exhibit surprisingly increased persistence and/or anti-tumor activity.

In one aspect, the engineered or transduced T-cells do not express an endogenous TCR(α/β). For example, in some aspects, prior to transduction with the cancer antigen specific TCR construct, the endogenous TCR(α/β) is knocked out using CRISPR technology, e.g., CRISPR/Cas9 technology (Legut, M., Dolton, G., Mian, A. A., Ottmann, O. G. & Sewell, A. K. CRISPR-mediated TCR replacement generates superior anticancer transgenic T cells. Blood 131, 311-322 (2018)), or CRISPR/Cas12a technology.

In one aspect, also disclosed herein are nucleic acids encoding an siRNA, e.g., shRNA for knocking down a gene for the enhancement of antitumor activity of TCR-transduced T cells in vivo. The nucleic acid sequences of shRNA stem target immune system negative signaling molecules, for example, checkpoint proteins and/or immune suppressor proteins. In some aspects, the shRNA targets may include, but are not limit to, programmed cell death protein (PD1), (Seq ID NO: 7), von Hippel-Lindau tumor suppressor (VHL) (Seq ID NO: 8), and/or protein phosphatase 2 regulatory subunit B delta (PPP2R2D) (Seq ID NO: 9). In some aspects, the PPP2R2D mRNA sequences which can be targeted can include or exclude portions or all of: PPP2R2D transcript variant 1 (SEQ ID NO: 18); or PPP2R2D, transcript variant 3 (SEQ ID NO: 19). In some embodiments, nucleic acids encoding an antisense RNA or DNA may also be used for knocking down a gene or reducing expression of a gene encoding a negative signaling molecule. In some aspects any of the nucleic acids of any of the preceding aspects or embodiments described herein may also comprise any of these nucleic acids encoding an siRNA/shRNA.

In one aspect, also disclosed herein are methods of stimulating an immunological response against a cancer or treating, inhibiting, and/or preventing a cancer, the method comprising administering to a subject a composition comprising a therapeutically effective amount of an epitope or TCR alpha or beta variable region of any preceding aspect or any aspect or embodiment disclosed herein and/or identified by the method of detecting or identifying epitope-specific T cells and TCRs of any preceding aspect or any aspect or embodiment disclosed herein.

In one aspect, this disclosure also features chimeric antigen receptors and T cells expressing a CAR. In some aspects, the CAR construct comprises an antigen recognition moiety (e.g., a single chain variable fragment (ScFv)), a transmembrane domain, and an intracellular T-cell activation moiety (consisting of CD28 or 4-1BB costimulatory signaling domain fused with a signaling domain, for example, a ZAP300 (Seq ID NO: 16) or ZAP327 (SEQ ID NO:17) signaling domain or other signaling domain derived from ZAP 70. In some aspects, the CARs may include or exclude, for example, antigen recognition moieties, e.g., ScFvs, that specifically bind to CD19, BCMA, B7-H3, Mesothelin or HER-2.

In another aspect, the one or a plurality of the TCR alpha and/or beta variable regions of any preceding aspect or any aspect or embodiment disclosed herein may also further comprise a ZAP300 or ZAP327 moiety or other signaling moiety derived from a ZAP70 kinase domain.

In one aspect this disclosure also relates to methods of using any TCR-T or CAR-T cells of any preceding aspect or any aspect or embodiment disclosed herein in the treatment of cancer.

Although T cell recognition of a target antigen is HLA-restricted, chimeric antigen receptor (CAR)-T cell recognition of a target is not HLA-dependent. As disclosed herein, the modulation of TCR-T cell and CAR-T signaling and function in vivo is critically important to prolong T cell persistence (and reduce T cell exhaustion) by direct regulation of TCR or CAR signaling domains or knockdown/knockout of negative signaling molecules which can include or exclude PD-1, VHL, PPP2R2D and epigenetic factors which can include or exclude JMJD3 and LSD1.

In one aspect, this disclosure also features methods and strategies to prolong TCR-T and CAR-T cell persistence by direct manipulation of TCR or CAR signaling domains or by knockdown/knockout of negative signaling molecules. In some aspects, this disclosure also feature methods to enhance CAR-T and TCR cell persistence by expression of chemokine receptor and shRNA knockout in TCR or CAR constructs. In some aspects the negative signaling molecules are, for example, indoleamine (2,3)-dioxygenase (IDO) (including isoforms IDO1 and IDO2), OX40, CTLA-4 (programmed cytotoxic T-lymphocyte antigen 4), PD-1 (programmed death 1), PD-L1 (programmed death ligand 1), PD-L2, lymphocyte activation gene 3 (LAG3), and B7 homolog 3 (B7-H3). In certain aspects, the negative signaling molecules are, for example, PD-1, VHL, PPP2R2D and epigenetic factors which can include or exclude JMJD3 and LSD1. In some aspects, treatment with any of the engineered TCR T cells of this disclosure, where the T cells comprise and/or express a signaling component and/or having negative signaling molecule knockdown surprisingly reduces relapse/cancer recurrence following initial treatment and initial reduction of the cancer/tumor burden..

In one aspect, this disclosure also features methods to enhance T cell trafficking into tumor cells in vivo by forced expression of chemokine receptors. In some aspects, expression of chemokine receptors is forced by fusing any of the CAR or TCR constructs of any of the preceding aspects or embodiments described herein withi a chemokine receptor. In some aspects the chemokine receptor is CCR5, CXCR3, and/or CCR2. In some aspects the chemokine receptor is CCR5.

In some aspects expression of the chemokine receptor and shRNA knockout can be used in any of the TCR or CAR construct in any of the preceding aspects or embodiments described herein.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the generation and characterization of single T cell clones from an HLA-DP4 presented NY-ESO-1 reactive T cell line. T cell clones were isolated from an HLA-DP4 presented NY-ESO-1 reactive cell line. After expansion of each T cell clone, the antigen recognition was screened using a peptide containing amino acids 157-170 of NY-ESO-1 (SEQ ID NO:1), presented by HLA-DP4-positive APCs.

FIG. 2 shows the map of construction of DP4-ESO-1 TCR from one T cell clone into pMSGV vector.

FIGS. 3A and 3B show the transduction of DP4-ESO-1 TCR in naïve CD4+ T cells. FIG. 3A shows DP4-ESO-1 TCR transduction efficiency in naïve CD4+ T cells was measured by TCR-specific antibody staining and flow cytometry. Naïve CD4+ T cells were transduced with retroviral supernatant produced from different DP4-ESO-1 TCR PG-13 clones. After the transduction, the expression of DP4-ESO-1 TCR was tested and the average transduction efficiency was 60-70%. FIG. 3B shows the functional test of DP4-ESO-1 TCR-transduced CD4+ T cells.

FIGS. 4A, 4B and 4C show the function characterization of DP4-ESO-1 TCR-T cells. FIG. 4A shows the T cell recognition against peptide. The DP4-ESO-1 TCR-T cells recognized NY-ESO-1 157-170 peptide presented by HLA-DP4+ cells. FIG. 4B shows the T cell recognition against naturally processed NY-ESO-1. The DP4-ESO-1 TCR-T cells recognized 293T cells transfected with full-length NY-ESO-1, HLA-DPA1 and HLA-DP4. FIG. 4C shows DP4-ESO-1 TCR functioned only in CD4+ T cells. Only CD4+ T cells, compared to CD8+ T cells, transduced by the DP4-ESO-1 TCR recognized NY-ESO-1 157-170, indicating the TCR function was limited in CD4+ T cells.

FIGS. 5A, 5B and 5C show the improvement of anti-tumor function in vivo by DP4-ESO-1 TCR when combined with A2-ESO-1 TCR against MDA-MB-231/DP4/ESO. FIG. 5A shows the migration of injected A2-ESO-1 TCR (labeled with luciferase) transduced CD8+ T cells in vivo was tracked by luciferase imaging. FIG. 5B shows the growth of MDA-MB-231/DP4/ESO in vivo treated by different groups of T cells were monitored. FIG. 5C shows the comparison of tumor sizes treated by different groups of T cells when mice were sacrificed.

FIGS. 6A, 6B, 6C and 6D show the generation and characterization of HLA-A2-restricted CT83-specific T cells. FIG. 6A shows in vitro peptide-stimulated T cells were generated and tested for their ability to recognize 293T/CT83 PEP90-98 (a peptide containing amino acids 90-98 of CT83 (SEQ ID NO:2), compared with 293T/control peptide. FIG. 6B shows A2-CT83-specific T cells recognized 293T cells transfected with Ii-CT83, CT83-GFP plasmid DNA or pulsed with CT83 PEPS-98 (a positive control), but did not recognize control 293T cells. FIG. 6C shows A2-CT83-specific T cells were tested for their ability to recognize human breast cancer cell line MDA-MB-231 (expressing HLA-A2 and CT83), but not MDA-MB-436 (HL-A2− CT83+). FIG. 6D shows the recognition of MDA-MB-231 cells could be blocked by anti-MHC I, but not anti-MHC II antibody.

FIGS. 7A and 7B show the vaccination with CT83-PEP90-98 inhibits breast cancer cells. FIG. 7A shows a schematic presentation of experimental design and schedule. FIG. 7B shows the tumor-bearing mice were treated by vaccination (i.v.) with TAT-CT83 PEP90-98-CMI nanoparticles, but not by TAT-CT83 PEP66-74-CMI. (CMI stands for CpG, MPLA and poly(I:C)). ** P value <0.01.

FIGS. 8A, 8B, 8C, 8D and 8E show the construction and characterization of HLA-A2-restricted CT83-specific TCR. FIG. 8A shows a flow chart of TCR sequencing, cloning and construction from the FACS-purified A2-CT83-specific T cell population using 10×single-cell barcoding technology and next-generation sequencing. FIG. 8B shows the functional analysis of A2-CT83 TCR-transduced T cells and vector transduced PBMCs against 293T, 293T/CT83-GFP, Cos-7 and Cos-7/Ii-CT83, Cos-7-A2/Ii-CT83 cells. FIG. 8C shows A2-CT83 TCR-T cells specifically recognized 293T pulsed with CT83 PEP90-98, compared with 293T cells pulsed with other CT83 peptides. FIG. 8D shows A2-CT83 TCR-T cells specifically recognized MDA-MB-231 cells (expressing CT83 and HLA-A2), but did not recognize cells which only expressed one of the polypeptides: MCF7 (A2+ CT83−), HTB-2 (A2+ CT83−) or MDA-MB-436 (A2− CT83+). FIG. 8E shows that A2-CT83 TCR-T cells were capable of recognizing CT83+ and HLA-A2+ lung cancer cells (HOP92/A2, NCI-H358/A and NCI-H838/A2), but not CT83+ HLA-A2− tumor cells (HOP92, NCI-H358 and NCI-838). These results demonstrate that A2-CT83 TCR is capable of specifically recognizing the naturally-processed CT83 epitope presented by HLA-A2 molecules.

FIGS. 9A, 9B and 9C show the antitumor activity of A2-CT83 TCR-T cells in vivo. FIG. 9A shows the diagram of the schedule of administration (injection) of NCI-H838 tumor cell cells and A2-CT83 TCR-T cells in a NSG mouse model. FIGS. 9B and 9C show the inhibition of tumor growth by A2-CT83 TCR-T cells in vivo. By contrast, tumor-bearing mice treated with control T cells developed large tumor masses. These studies suggest that A2-CT83 TCR-T cells have potent antitumor activity in vivo.

FIGS. 10A and 10B show the generation and characterization of HLA-A2-restricted HCMV (pp65 and IE-1 proteins)-specific T cell clones. FIG. 10A shows that seven T cell clones reactive to pp65 (495-503) and five T cell clones reactive to IE-1 (316-324) were picked and screened with peptide-pulsed T2 cell and expanded in vitro. FIG. 10B shows the characterization of HLA restriction of pp65 T cell clone #3 and IE-1 T cell clone #5. Both T cell clones could specifically recognize the HLA-A2-presented pp65 and IE-1 antigens in Cos-7 cells, respectively.

FIGS. 11A, 11B, 11C, 11D and 11E show the identification and cloning and characterization of HLA-A2-restricted pp65 and IE-1-specific TCRs and their function of TCR-transduced human T cells. FIG. 11A shows the transduction efficiency of A2-pp65 TCR and A2-IE-1 TCR in human CD8+ T cells isolated from HCMV seronegative donors. FIG. 11B shows A2-pp65 TCR-transduced and A2-IE-1 TCR-transduced T cells specifically recognized glioblastoma cells expressing HLA-A2 and HCMV antigens (pp65 or IE-1) or glioblastoma infected by HCMV. FIG. 11C shows dose-dependent recognition of A2-pp65 TCR-transduced and A2-IE-1 TCR-transduced T cells against T2 cells pulsed with pp65 (495-503) or IE-1(316-324) peptides, respectively. FIG. 11D shows that A2-pp65 TCR-transduced and A2-IE-1 TCR-transduced T cells specifically killed glioblastoma cells expressing HLA-A2 and HCMV antigens (pp65 or IE-1) or glioblastoma infected by HCMV. FIG. 11E shows dose-dependent cytotoxicity of HCMV/AD169-infected U87 tumor cells by A2-pp65 TCR- and A2-IE-1 TCR-transduced T cells.

FIGS. 12A, 12B, 12C and 12D show the antitumor activity of A2-pp65 TCR-T cells in vivo. A transplanted tumor model was established in immunodeficient mice using U87 cells expressing pp65 or IE-I, along with luciferase. Tumor cells were grown in SCID/Beige for 3 days and then treated by adoptive transfer of A2-pp65 TCR, A2-IE-1 TCR or control TCR transduced human T cells by i.v. injection of 2×10⁶ T cells per mouse. FIG. 12A shows the diagram of the procedure of in vivo functional analysis of A2-pp65 TCR-T cells. FIG. 12B show the migration of A2-pp65 TCR-T cells after injection into the tumor-bearing mice. FIG. 12C shows that A2-pp65 TCR-T cells specifically inhibited the tumor growth of U87 expressing pp65 tumors in vivo. FIG. 12D shows that tumor weight was dramatically decreased after the treatment with A2-pp65 TCR-T cells, suggesting the potential antitumor activity of pp65 TCR-T cells in the treatment of glioblastoma.

FIGS. 13A, 13B, 13C and 13D show the antitumor activity of A2-IE-1 TCR-T cells in vivo. FIG. 13A shows the diagram of the procedure of in vivo functional analysis of A2-IE-1 TCR-T cells. FIG. 13B show the migration of A2-IE-1 TCR-T cells after injection into the tumor-bearing mice. FIG. 13C shows that A2-IE-1 TCR-T cells specifically inhibited the tumor growth of U87 expressing IE-1 tumors in vivo. FIG. 13D show the tumor weight was dramatically reduced after the treatment with A2-IE-1 TCR-T cells, suggesting the potential antitumor activity of A2-IE-1 TCR-T cells in the treatment of glioblastoma.

FIGS. 14A, 14B, 14C, 14D, 14E, 14F and 14G show the enhancement of A2-ESO-1 TCR-T cell surface expression and function in human T cells by mouse constant sequence. FIG. 14A shows the schematic diagram of conventional and modified A2-ESO-1 TCR constructs of the present disclosure. FIG. 14B shows that a modified TCR of the present disclosure has higher transduction efficiency than conventional TCR construct. FIG. 14C shows that TCR cell surface expression was detected by FACS. FIG. 14D shows that TCR cell surface expression was detected by confocal microscopy. FIG. 14E shows the results of an LDH assay to detect the target cell killing ability of conventional and modified TCR-T cells. FIG. 14F shows that cytokine secretion was detected by ELISA after co-cultured with A2-ESO-1 positive breast cancer cells. FIG. 14G shows the long-term tumor cell killing ability of representative compositions of the present disclosure was detected by in-vitro co-culture.

FIGS. 15A, 15B, 15C and 15D show that modified A2-ESO-1 specific TCR-T cells have better therapeutic efficiency in a pre-clinical breast cancer model. FIG. 15A shows the schematic diagram of an animal experiment. 1×10⁶ cells of NY-ESO-1 positive breast cancer line MDA-MB-231 (ESO-1+) were subcutaneously injected to NSG fat pad. A2-ESO-1 TCR-T/A2-ESO-1 TCR-M-T cells were intravenously injected to tumor bearing mice and followed with 3 doses of IL-2. FIG. 15B shows the tracking of tumor growth. FIG. 15C shows the tumor images after sacrifice. FIG. 15D shows the tumor weight after sacrifice.

FIGS. 16A, 16B and 16C show the in vitro tumor killing of A2-ESO-1 TCR with amino acid substitutions and murine TCR constant sequences. FIG. 16A shows that five substitutions of A2-ESO-1 TCRs and original A2-ESO-1 TCR that were transduced in human T cells and tested for their ability to recognize tumor cells with or without HLA-A2 and NY-ESO-1 expression. FIG. 16B shows the cytotoxicity of substituted A2-ESO-1 TCR-T cells against tumor cells with or without HLA-A2 and NY-ESO-1 expression. S2 and S5 of A2-ESO-1 TCR transduced T cells showed higher cytolysis activity. FIG. 16C shows the human TCR constant regions of S2 and S5 of A2-ESO-1 TCR and original A2-ESO-1 were substituted with murine TCR constant regions. S2 of A2-ESO-1 TCR with murine TCR constant region sequence showed potent T cell response.

FIGS. 17A, 17B and 17C show the antitumor activity of A2-CT83 TCR-M-T cells (murine constant regions). FIG. 17A shows the transduction efficiency of A2-CT83 TCR-M in human T cells. FIG. 17B shows A2-CT83 TCR-M-T cells specifically recognized MDA-MB-231 and NCI-H1563 cells (expressing CT83 and HLA-A2), but not CAMA-1 cells (A2+ CT83−). FIG. 17C shows the cytotoxicity of A2-CT83 TCR-M-T cells with MDA-MB-231 and NCI-H1563 cells. These results indicate that A2-CT83 TCR-M T cells are potent and specific against tumor cells, with reduced TCR mispairing.

FIGS. 18A, 18B, 18C, 18D and 18E show novel CAR-T constructs fused with ZAP300 and ZAP327 derived from ZAP70 and their functional comparison with conventional CAT-T construct containing a CD3-t signaling domain. FIG. 18A shows the schematic presentation of conventional CD19-CD28-CD3z (1928z), and novel constructs comprising CD19-CD28-ZAP300 (1928ZAP300) and anti-CD19-CD28-ZAP327 (1928ZAP327). FIG. 18B shows the T cell transduction efficiency of three kinds of CAR in human T cells. FIGS. 18C and 18D show the antigen-specific recognition and tumor cell lysis after CAR-T cells cocultured with Raji tumor cells. FIG. 18E shows the in vivo antitumor activity of three kinds of CAR-T cells (1928z, 1928ZAP300 and 1928ZAP327). Importantly, 1928ZAP300 and 1928ZAP327 CAR-T cells outperformed 1928z CAR-T cells in vivo experiments, and markedly prolonged overall mouse survival in a Raji lymphoma tumor model. These studies suggest that replacing CD3ζ chain with Zap70 kinase domains (ZAP300 and ZAP327) markedly enhances antitumor activity in vivo.

FIGS. 19A, 19B, 19C, 19D and 19E show that novel 4-1BB-contain CAR-T constructs fused with ZAP300 and ZAP327 derived from ZAP70 produced low amounts of cytokines but more potent antitumor immunity. FIG. 19A shows the schematic construction of 19bbz and 19bbZAP327. FIG. 19B shows that 19bbZAP327 CAR-T cells produced significantly lower amounts of cytokines compared with conventional 19bbz CAR-T cells after stimulation with tumor cells. FIG. 19C shows specific lysis of tumor cells by 19bbZAP327 CAR-T cells. FIGS. 19D and 19E show that 19bbZAP327 CAR-T cells had superior antitumor activity in vivo and markedly prolonged mouse survival, suggesting that 19bbZAP327 CAR-T cells improve the safety and antitumor immunity, compared with conventional 19bbz CAR-T cells.

FIGS. 20A, 20B and 20C show that ZAP327 signaling domain promotes T cell memory function and persistence in vivo. FIG. 20A shows higher in vivo persistence of 1928ZAP327 CAR-T cells in bone marrows and spleens of the T cell transferred mice. FIG. 20B shows higher percentages of central memory 1928ZAP327 CAR-T cells, compared with 1928z CAR-T cells. FIG. 20C shows that 1928ZAP327 CAR-T cells expressed lower amount of PD-1 (an exhaustion marker) than 1928z CAR-T cells, suggesting that ZAP327 signaling domain reduces T cell exhaustion.

FIGS. 21A and 21B show the modulation of TCR-T cell function in vivo by knocking down the expression of metabolic genes PD1, VHL, PPP2R2D. FIG. 21A shows the transduction efficiency of A2-ESO-1 TCR constructs with or without PD1, VHL or PPP2R2D shRNA respectively. FIG. 21B shows the MDA-MB-231/A2/NY-ESO-1 bearing mice were injected with A2-ESO-1 TCR-T cells with or without PD1, VHL or PPP2R2D knockdown respectively. Upper: Average tumor growth in each group. Middle: Mice survival curve in each group. Lower: Tumor growth in each mouse of each group.

FIG. 22 shows the enhancement of enhanced CD44+CD62L− memory T cell population by Jmjd3 conditional knockout (cKO) in CD4+ T cells, compared with wild-type (WT) cells.

FIGS. 23A, 23B, 23C, 24D, 25E and 25F show the enhancement of T cell survival and persistence in vivo and in vitro by Jmjd3 cKO T cells. FIGS. 23A and 23B show that CD4+ T cells from Jmjd3 cKO 2d2 transgenic mice stimulated in vivo with MOG peptide plus complete Freund's adjuvant markedly enhanced clinical scores in an EAE mouse model. FIG. 23C shows higher numbers of Jmjd3 cKO T cells, compared with wildtype 2d2 cells after T cell transfer. FIGS. 23D, 23E and 23F show higher numbers of Jmjd3 cKO T cells, compared with wildtype 2d2 cells after T cell transfer using T cells stimulated with MOG peptide in vitro.

FIGS. 24A, 24B and 24C show that Jmjd3 KO enhances T cell survival and persistence by reducing T cell apoptosis. FIG. 24A shows that Jmjd3 cKO T cells reduced apoptosis-related protein levels after stimulation with anti-CD3 and CD28 antibodies. FIG. 24B shows Jmjd3 cKO T cells had much lower levels of T cell apoptosis after stimulation. FIG. 24C shows Jmjd3 cKO T cells had very low level of cleaved Caspase 3, compared with WT T cells.

FIGS. 25A, 25B, 25C and 25D show the enhancement of CAR-T cell survival and persistence in vivo by Jmjd3 knockdown (KD). FIG. 25A shows experiment design using Raji tumor cells for monitoring luciferase-labeled T cell survival. FIGS. 25B and 25C show that CAR-T cells with Jmjd3 KD (1928z-shJMJD3) had strong proliferation at day 4 after T cell transfer into Raji tumor-bearing NSG mice, but maintained high levels of T cells, compared to 1928z-control shRNA. FIG. 25D shows that 1928z-shJMJD3 CAR-T cells markedly inhibited tumor growth and prolonged mouse survival, compared to 1928z-control shRNA CAR-T cells with control shRNA.

FIGS. 26A, 26B and 26C show the enhancement of T cell trafficking into tumor cells in vivo by forced expression of chemokine receptors. FIG. 26A shows the diagram of construction of 1928z CAR fused with CCR5. FIGS. 26B and 26C show that 1928z-CCR5 CAR-T cells markedly inhibited the growth of MDA-MB-231/CD19 tumor cells in vivo, suggesting that forced chemokine receptor expression enhances T cell trafficking into tumor cells.

FIG. 27 illustrates a strategy to enhance both T cell trafficking and T cell persistence by expression of chemokine receptor and shRNA KD in TCR or CAR constructs.

### DETAILED DESCRIPTION

### A. Definitions

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed. It is also understood that whenever a series of values are disclosed, that any range falling between any two of the recited values is also understood by one of skill in the art,

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

As used herein, the term “antibody” includes both polyclonal and monoclonal antibodies; primatized (e.g., humanized); murine; mouse-human; mouse-primate; and chimeric; and may be an intact molecule, a fragment thereof (which can include or exclude scFv, Fv, Fd, Fab, Fab′ and F(ab)′2 fragments), or multimers or aggregates of intact molecules and/or fragments; and may occur in nature or be produced, e.g., by immunization, synthesis or genetic engineering; an “antibody fragment,” as used herein, refers to fragments, derived from or related to an antibody, which bind antigen and which in some embodiments may be derivatized to exhibit structural features that facilitate clearance and uptake, e.g., by the incorporation of galactose residues. “Antibodies” includes, e.g., F(ab), F(ab)′2, scFv, light chain variable region (VL), heavy chain variable region (VH), and combinations thereof.

Checkpoint inhibiting agents are agents that target checkpoint proteins or a derivative thereof, and may be referred to as “checkpoint inhibitors.” Checkpoint inhibitors can include or exclude proteins, polypeptides, amino acid residues, and monoclonal or polyclonal antibodies. The polyvalent vaccine can include or be administered along with one or more checkpoint inhibitor. The checkpoint inhibitors can bind, for example, to ligands or proteins that are found on any of the family of T cell regulators, such CD28/CTLA-4. Targets of checkpoint inhibitors include, but are not limited to, receptors or co-receptors (e.g., CTLA-4; CD8) expressed on immune system effector or regulator cells (e.g., T cells); proteins expressed on the surface of antigen-presenting cells (e.g., expressed on the surface of activated T cells, including PD-1, PD-2, PD-L1 PD-L2, 4-1BB, and OX40); metabolic enzymes or metabolic enzymes that are expressed by both tumor and tumor-infiltrating cells (e.g., indoleamine (IDO), including isoforms, such as IDO1 and IDO2); proteins that belong to the immunoglobulin superfamily (e.g., lymphocyte-activation gene 3, also known as LAG3); proteins that belong to the B7 superfamily (e.g., B7-H3 or homologs thereof). B7 proteins can be found on both activated antigen presenting cells and T cells.

As used herein, the term “separation”, includes any means of substantially purifying one component from another (e.g., by filtration, magnetic attraction, etc.).

As used herein, the term “isolation” or “isolating”, includes any means of sorting one species of a type of genus from another species of a type of genus.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The terms “preventing” or “inhibiting” the development of a cancer or cancer cells” as used herein, refers to the occurrence of the cancer being prevented or the onset of the cancer being delayed.

The term “treating” or “reducing the presence of a cancer or cancer cells” as used herein, means that the cancer growth is inhibited, which is reflected by, e.g., tumor volume or numbers of malignant cells. Tumor volume may be determined by various known procedures, e.g., measuring the observed image and comparing the average cross-sectional diameter of the tumor with a calibration line (e.g., as done in ImageJ).

“Preventing or inhibiting the development of an infectious disease” as used herein, means the occurrence of the infectious disease is prevented or the onset of the infectious disease is delayed, or the spread of an existing infection is reversed.

As used herein, the term “activation”, refers to the state of a cell following sufficient cell surface moiety ligation to induce a noticeable biochemical or morphological change. Within the context of T-cells, such activation, refers to the state of a T-cell that has been sufficiently stimulated to induce cellular proliferation. Activation of a T-cell may also induce cytokine production and performance of regulatory or cytolytic effector functions. Within the context of other cells, this term infers either up or down regulation of a particular physico-chemical process.

As used herein, the terms “cancer antigen” or “tumor antigen” encompasses tissue-specific differentiation antigens, tumor-specific shared antigens, and mutated tumor-specific and unique antigens, and any portion, peptide, or polypeptide of those antigens capable of eliciting an immune response of CD4+ or CD8+ T cells. Tumor antigens or cancer antigens that are recognized by CD8+ or CD4+ T cells can be classified into several categories (Wang, R. F. & Wang, H. Y. Immune targets and neoantigens for cancer immunotherapy and precision medicine. Cell research 27, 11-37, doi:10.1038/cr.2016.155 (2017)): 1) The tissue-specific differentiation antigens, which include MART-1 (Kawakami, Y. et al. Identification of the immunodominant peptides of the MART-1 human melanoma antigen recognized by the majority of HLA-A2-restricted tumor infiltrating lymphocytes. J. experimental medicine 180, 347-352 (1994); Schneider, J., Brichard, V., Boon, T., Meyer zum Buschenfelde, K. H. & Wolfel, T. Overlapping peptides of melanocyte differentiation antigen Melan-A/MART-1 recognized by autologous cytolytic T lymphocytes in association with HLA-B45.1 and HLA-A2.1. International journal of cancer 75, 451-458 (1998), TRP-1/gp75 (Wang, R. F., Parkhurst, M. R., Kawakami, Y., Robbins, P. F. & Rosenberg, S. A. Utilization of an alternative open reading frame of a normal gene in generating a novel human cancer antigen. J. experimental medicine 183, 1131-1140 (1996)), TRP-2 (Wang, R. F., Appella, E., Kawakami, Y., Kang, X. & Rosenberg, S. A. Identification of TRP-2 as a human tumor antigen recognized by cytotoxic T lymphocytes. J. experimental medicine 184, 2207-2216 (1996); Parkhurst, M. R. et al. Identification of a shared HLA-A*0201-restricted T-cell epitope from the melanoma antigen tyrosinase-related protein 2 (TRP2). Cancer research 58, 4895-4901 (1998); Sun, Y. et al. Identification of a new HLA-A(*)0201-restricted T-cell epitope from the tyrosinase-related protein 2 (TRP2) melanoma antigen. International journal of cancer 87, 399-404 (2000)), and gp100 (Kawakami, Y. et al. Recognition of multiple epitopes in the human melanoma antigen gp100 by tumor-infiltrating T lymphocytes associated with in vivo tumor regression. J. immunology 154, 3961-3968 (1995); Bakker, A. B. et al. Melanocyte lineage-specific antigen gp100 is recognized by melanoma-derived tumor-infiltrating lymphocytes. J. experimental medicine 179, 1005-1009 (1994); Skipper, J. C. et al. Shared epitopes for HLA-A3-restricted melanoma-reactive human CTL include a naturally processed epitope from Pmel-17/gp100. J. Immunol. 157, 5027-5033 (1996); Tsai, V. et al. Identification of subdominant CTL epitopes of the GP100 melanoma-associated tumor antigen by primary in vitro immunization with peptide-pulsed dendritic cells. J. Immunol. 158, 1796-1802 (1997)) have higher expression in cancer cells compared with normal cells; 2) Tumor-specific shared antigens which can include or exclude MAGE-A1 (Traversari, C. et al. A nonapeptide encoded by human gene MAGE-1 is recognized on HLA-A1 by cytolytic T lymphocytes directed against tumor antigen MZ2-E. J. experimental medicine 176, 1453-1457 (1992); Fujie, T. et al. A MAGE-1-encoded HLA-A24-binding synthetic peptide induces specific anti-tumor cytotoxic T lymphocytes. International journal of cancer 80, 169-172 (1999)). and NY-ESO-1 (Jager, E. et al. Simultaneous humoral and cellular immune response against cancer-testis antigen NY-ESO-1: definition of human histocompatibility leukocyte antigen (HLA)-A2-binding peptide epitopes. J. experimental medicine 187, 265-270 (1998); Rimoldi, D. et al. Efficient simultaneous presentation of NY-ESO-1/LAGE-1 primary and nonprimary open reading frame-derived CTL epitopes in melanoma. J. Immunol. 165, 7253-7261 (2000); Valmori, D. et al. Naturally occurring human lymphocyte antigen-A2 restricted CD8+ T-cell response to the cancer testis antigen NY-ESO-1 in melanoma patients. Cancer research 60, 4499-4506 (2000); Wang, R.-F., Johnston, S. L., Zeng, G., Schwartzentruber, D. J. & Rosenberg, S. A. A breast and melanoma-shared tumor antigen: T cell responses to antigenic peptides translated from different open reading frames. J. Immunol. 161, 3596-3606 (1998)) are expressed in cancer and testis, but not in other normal tissues. These antigens are also called cancer-testis (CT) antigens; 3) Tumor-specific and unique antigens that are mutated antigens, including CDK4 (Wolfel, T. et al. A p16INK4a-insensitive CDK4 mutant targeted by cytolytic T lymphocytes in a human melanoma. Science 269, 1281-1284 (1995)), catenin (Robbins, P. F. et al. A mutated beta-catenin gene encodes a melanoma-specific antigen recognized by tumor infiltrating lymphocytes. J. experimental medicine 183, 1185-1192 (1996)), and caspase-8 (Mandruzzato, S., Brasseur, F., Andry, G., Boon, T. & van der Bruggen, P. A CASP-8 mutation recognized by cytolytic T lymphocytes on a human head and neck carcinoma. J. experimental medicine 186, 785-793 (1997)) antigens; and 4) Overexpressed tumor antigens that are overexpressed in cancer cells compared with in normal cells.

Two cancer-testis (CT) antigens, NY-ESO-1, encoded by CTAG1B gene, and CT83 (also known as KK-LC-1), encoded by CT83 gene, are widely expressed in a variety of tumors including lung cancer and breast cancer. Both CD8+ T cells and antibodies have been shown to recognize NY-ESO-1, and a clinical response of 50-80% using NY-ESO-1-specific TCR has been demonstrated in several solid cancers, including melanoma, sarcoma and myeloma. Despite the importance of CD4+ T cells (HLA-DP4 is the most frequent HLA II molecule expressing in general human population, accounting for 70% positive), HLA-DP4-restricted NY-ESO-1 specific TCRs have not been tested in a clinical setting. The inventors previously identified HLA-DR4- and HLA-DP4-restricted NY-ESO-1 epitopes (Zeng, G. et al. Identification of CD4+ T cell epitopes from NY-ESO-1 presented by HLA-DR molecules. J. Immunol. 165, 1153-1159 (2000); Zeng, G., Wang, X., Robbins, P. F., Rosenberg, S. A. & Wang, R.-F. CD4+ T cell recognition of MHC class II-restricted epitopes from NY-ESO-1 presented by a prevalent HLA-DP4 allele: association with NY-ESO-1 antibody production. Proc. Natl. Acad. Sci. U.S.A. 98, 3964-3969 (2001)), and showed that an HLA-DP4-NY-ESO-1 peptide overlapped with an HLA-A2-restricted NY-ESO-1 peptide. Zeng, G. et al. Generation of NY-ESO-1-specific CD4+ and CD8+ T cells by a single peptide with dual MHC class I and class II specificities: a new strategy for vaccine design. Cancer Res. 62, 3630-3635. (2002).

As disclosed herein, HLA-DP4 restricted NY-ESO-1 CD4+ T cells and TCRs were generated, and tested for whether the combination of DP4-ESO-1 TCR-engineered T cells with A2-ESO-1 TCR-engineered T cells could generate stronger antitumor immunity than either alone.

In addition to the CT antigen NY-ESO-1, CT83 is highly expressed in 60-70% of breast cancer, particularly in TNBC, which is in consistent with previous reports. Fukuyama, T. et al. Identification of a new cancer/germline gene, KK-LC-1, encoding an antigen recognized by autologous CTL induced on human lung adenocarcinoma. Cancer research 66, 4922-4928, doi:10.1158/0008-5472.CAN-05-3840 (2006); Paret, C. et al. CXorf61 is a target for T cell based immunotherapy of triple-negative breast cancer. Oncotarget 6, 25356-25367, doi:10.18632/oncotarget.4516 (2015). However, relatively little was known about its immunogenicity, T cell epitopes and the cognate TCRs for tumor recognition by T cells. As disclosed herein antigen-specific CD4+ and CD8+ T cells were generated, and used to identify HLA-A2-restricted CT83-specific TCR (A2-CT83 TCR) to determine if CT83 could serve as an attractive target for TCR-T cell immunotherapy.

It has been demonstrated that CAR-T and TCR-T cell persistence is closely correlated with patient survival. Thus, modulation of TCR-T and CAR-T-cell signaling may enhance T cell persistence and reduce T cell exhaustion through direct regulation of CAR or TCR signaling and knockdown or knockout of negative signaling molecules which can include or exclude PD1, VHL, PPP2R2D and epigenetic factors which can include or exclude Jmjd3 and LSD1.

In some embodiments, the immunogenetic peptides and epitopes contained within the tumor antigens of the present disclosure are derived from the NY-ESO-1 protein and CT83 protein, which are both expressed widely in various types of cancer, including but not limiting to breast cancer, lung cancer, prostate cancer, etc. The tumor antigens of the present invention are expressed with significantly high levels in tumor cells and testis, compared to low levels in normal cells.

In some embodiments, the “tumor antigen” or “cancer antigen” is the NY-ESO-1, CT83 protein, HCMV pp65 protein and/or HCMV 1E-1 protein and any portion, peptide, or polypeptide of the NY-ESO-1, CT83 protein, HCMV pp65 protein and/or HCMV 1E-1 protein capable of eliciting an immune response of CD4+ or CD8+ T cells, including the full-length NY-ESO-1 and CT83 proteins.

The “immunogenetic peptides and epitope” as the term is used herein, encompasses any epitope or fragment of NY-ESO-1, CT83, HCMV pp65 and/or HCMV IE-1 proteins which act as tumor antigens.

“Fragment” or “portion” as the term is used herein means any segment of a protein or gene, having at least 5 or 6 amino acids in the case of a protein fragment and at least 15-18 nucleotides in the case of a gene.

In one embodiment, tumor antigen-specific T cell line of the invention comprises all CD4+ or CD8+ T lymphocytes generated that immunologically recognize the tumor antigen presented by antigen-presenting cells that are HLA-DP4 or HLA-A2 positive.

The “presented” as the term is used herein, encompasses the procedure of transfection of the DNA encoding the full-length or any portion of the tumor antigen into antigen-presenting cells or loading of the peptide of the full-length or any portion of the tumor antigen onto antigen-presenting cells.

The “antigen-presenting cell” as the term is used herein, encompasses any natural or artificial cell line or cell that express a certain kind of HLA molecule of interest on the cell surface. As used herein, the term “antigen” refers to any molecule 1) capable of being specifically recognized, either in its entirety or fragments thereof, and bound by the “idotypic” portion (antigen-binding region) of a mAb or its derviative; 2) containing peptide sequences which can be bound by MHC and then, in the context of MHC presentation, can specifically engage its cognate T cell antigen receptor.

The “HLA-DP4 positive” as the term is used herein, encompasses any natural or artificial cell line or cell that express HLA class II molecules DPA1 and DPB1*04 including all its subtypes on the cell surface.

The “HLA-A2 positive” as the term is used herein, encompasses any natural or artificial cell line or cell that express HLA class I molecule A*02 including all its subtypes on the cell surface.

In one embodiment of the present invention, at least two T cell receptors are derived from the antigen-specific CD4+ or CD8+ T cell line. The full-length alpha chain and beta chain of the TCR are cloned separately. The “full length” as the term is used herein, encompasses an alpha chain variable region fused with a human alpha chain constant region (as a non-limiting example, TRAC, IQNPDPAVYQLRDSKSSDKSVCLFTDFDSQTNVSQSKDSDVYITDKTVLDMRSMDFK SNSAVAWSNKSDFACANAFNNSIIPEDTFFPSPESSCDVKLVEKSFETDTNLNFQNLS VIGFRILLLKVAGFNLLMTLRLWSS) (Seq ID NO: 10), or murine alpha chain constant region (trac) (SEQ ID NO:13)) or a beta chain variable region fused with human beta chain constant region type2 (TRBC2, DLKNVFPPEVAVFEPSEAEISHTQKATLVCLATGFYPDHVELSWWVNGKEVHSGVST DPQPLKEQPALNDSRYCLSSRLRVSATFWQNPRNHFRCQVQFYGLSENDEWTQDRA KPVTQIVSAEAWGRADCGFTSESYQQGVLSATILYEILLGKATLYAVLVSALVLMAM VKRKDSRG) (Seq ID NO: 12), or a human beta chain constant region type 1 (TRBC1, DLNKVFPPEVAVFEPSEAEISHTQKATLVCLATGFFPDHVELSWWVNGKEVHSGVST DPQPLKEQPALNDSRYCLSSRLRVSATFWQNPRNHFRCQVQFYGLSENDEWTQDRA KPVTQIVSAEAWGRADCGFTSVSYQQGVLSATILYEILLGKATLYAVLVSALVLMA MVKRKDF) (SEQ ID NO:11), or murine beta chain constant region type 1 (trbc1) (SEQ ID NO:14), or murine beta chain constant region type 2 (trbc2) (SEQ ID NO:15). In some embodiments, the constant region may have a sequence having 85, 86, 87, 88, 89, 90, 91 92 93, 94, 95, 96, 97, 98, or 99% sequence identity to SEQ ID NO:10, SEQ ID NO:11, SEQ ID NO:12, SEQ ID NO:13, SEQ ID NO:14, or SEQ ID NO:15)). The constant region may also contain 1, 2, 3, 4, 5, 6, 7, 8, 9, or 10 substitutions. In some embodiments the substitutions will be conservative substitutions.

In some embodiments, the chimeric TCR is a CT83 specific TCR having a chimeric alpha chain comprising a HLA-A2-restricted CT83 TCR alpha chain variable domain fused with murine alpha constant domain comprising SEQ ID NO:20 or a variant thereof having 85, 86, 87, 88, 89, 90, 91 92 93, 94, 95, 96, 97, 98, or 99% sequence identity to SEQ ID NO:20 and/or 1, 2, 3, 4, 5, 6, 7, 8, 9, or 10 substitutions to SEQ ID NO:20, which may be conservative substitutions; and a chimeric beta chain comprising a HLA-A2-restricted CT83 TCR beta chain variable domain fused with murine Beta constant domain 2 having SEQ ID NO:21 or a variant thereof having 85, 86, 87, 88, 89, 90, 91 92 93, 94, 95, 96, 97, 98, or 99% sequence identity to SEQ ID NO:21 and/or 1, 2, 3, 4, 5, 6, 7, 8, 9, or 10 substitutions to SEQ ID NO:21, which may be conservative substitutions,

In some embodiments, the chimeric TCR is a NY-ESO-1 specific TCR having a chimeric alpha chain comprising a polypeptide selected from a HLA-A2-restricted NY-ESO-1 TCR (S2) alpha chain variable domain fused with murine alpha constant domain selected from a polypeptide having SEQ ID NO: 22 or a variant thereof having 85, 86, 87, 88, 89, 90, 91 92 93, 94, 95, 96, 97, 98, or 99% sequence identity to SEQ ID NO:22 and/or 1, 2, 3, 4, 5, 6, 7, 8, 9, or 10 substitutions to SEQ ID NO:22, which may be conservative substitutions, and a HLA-A2-restricted NY-ESO-1 TCR (S5) alpha chain variable domain fused with a murine alpha constant domain having SEQ ID NO:24 or a variant thereof having 85, 86, 87, 88, 89, 90, 91 92 93, 94, 95, 96, 97, 98, or 99% sequence identity to SEQ ID NO:24 and/or 1, 2, 3, 4, 5, 6, 7, 8, 9, or 10 substitutions to SEQ ID NO:24, which may be conservative substitutions; and a chimeric beta chain selected from a HLA-A2-restricted NY-ESO-1 TCR (S2) (G50A, A51E) Beta chain variable domain fused with murine Beta constant domain 2 comprising SEQ ID NO:23 or a variant thereof having 85, 86, 87, 88, 89, 90, 91 92 93, 94, 95, 96, 97, 98, or 99% sequence identity to SEQ ID NO:23 and/or 1, 2, 3, 4, 5, 6, 7, 8, 9, or 10 substitutions to SEQ ID NO:23, which may be conservative substitutions, and a HLA-A2-restricted NY-ESO-1 TCR (S5) (G50A, A51E, A97L) Beta chain variable domain fused with murine Beta constant domain 2 having SEQ ID NO:25 or a variant thereof having 85, 86, 87, 88, 89, 90, 91 92 93, 94, 95, 96, 97, 98, or 99% sequence identity to SEQ ID NO:25 and/or 1, 2, 3, 4, 5, 6, 7, 8, 9, or 10 substitutions to SEQ ID NO:25.

In some embodiments, the chimeric TCR comprises a sequence having 85, 86, 87, 88, 89, 90, 91 92 93, 94, 95, 96, 97, 98, or 99% sequence identity to SEQ ID NO:10, SEQ ID NO:11, SEQ ID NO:12, SEQ ID NO:13, SEQ ID NO:14, or SEQ ID NO:15)). The constant region may also contain 1, 2, 3, 4, 5, 6, 7, 8, 9, or 10 substitutions. In some embodiments the substitutions will be conservative substitutions.

The term “proliferation” as used herein, means to grow or multiply by producing new cells.

The terms “purify” or “pure” refer to a molecule isolated from other reaction or cellular components. The term “substantially pure” or “substantially purified” refers to a molecule which is 85, 86, 87, 88, 89, 90, 91, 9, 93, 94, 95, 96, 97, 98, 99, or 100% pure.

In yet another embodiment of the present invention, epitopes of the tumor antigen that interact specifically with the CD4+ T cell line or the TCR to elicit T cell immune response include or exclude but are not limited to NY-ESO-1 PEP161-180 (WITQCFLPVFLAQPPSGQRR, Seq ID NO:34), NY-ESO-1 PEP156-175 (LSLLMWITQCFLPVFLAQPP, Seq ID NO:35), NY-ESO-1 PEP157-170 (SLLMWITQCFLPVF, Seq ID NO:1), which are peptides/portions of NY-ESO-1 protein, containing the specified amino acids of the NY-ESO-1 protein (e.g., PEP 161-180 comprises amino acids 161-180 of the NY-ESO-1 protein). In some embodiments, the epitopes include variants comprising 1, 2 or 3 conservative substitutions. NY-ESO-1 PEP161-180 (Seq ID NO:34) has been identified as a peptide with high affinity to HLA-DP4. Zeng, G. et al. Identification of CD4+ T cell epitopes from NY-ESO-1 presented by HLA-DR molecules. J. Immunol. 165, 1153-1159 (2000). The usage of this peptide has generated peptide-stimulated CD4+ T cells that specifically recognize HLA-DP4 presented NY-ESO-1. Zeng, G., Wang, X., Robbins, P. F., Rosenberg, S. A. & Wang, R. F. CD4(+) T cell recognition of MHC class II-restricted epitopes from NY-ESO-1 presented by a prevalent HLA DP4 allele: association with NY-ESO-1 antibody production. Proc. Natl Acad Sci USA 98, 3964-3969, doi:10.1073/pnas.061507398 (2001). NY-ESO-1 PEP157-170 (Seq ID NO:1) has been identified as the shortest functional epitope which maintains an unweakened immune response compared to full length NY-ESO-1.⁴² Id. (Zeng et al. PNAS doi:10.1073/pnas.061507398 (2001)).

In yet another embodiment of the present invention, epitopes of the tumor antigen that interact specifically with the CD8+ T cell line or the TCR to elicit T cell immune response can include or exclude but are not limited to CT83 PEP90-98 (KLVELEHTL, Seq ID NO:2), CT83 PEP6-14 (LLASSILCA, Seq ID NO:36), CT83 PEP4-12 (YLLLASSIL, Seq ID NO37:), CT83 PEP79-87 (RILVNLSMV, Seq ID NO:38), CT83 PEP10-31 (SILCALIVFWKYRRFQRNTGEM, Seq ID NO:39), CT83 PEP66-76 (ILNNFPHSIAR, Seq ID NO:40), which are peptides/portions of the CT83 protein, containing the specified amino acids of the CT83 protein. In some embodiments, the epitopes include variants comprising 1, 2 or 3 conservative substitutions.

In yet another embodiment of the present invention, epitopes of the tumor antigen that interact specifically with the CD4+ T cell line or the TCR to elicit T cell immune response include or exclude but are not limited to pp65 peptide (495-503) (NLVPMVATV, SEQ ID NO:26). pp65 is a HCMV protein and an antigen expressed by glioblastoma cells. In some embodiments, the epitopes include variants comprising 1, 2 or 3 conservative substitutions.

In yet another embodiment of the present invention, epitopes of the tumor antigen that interact specifically with the CD4+ T cell line or the TCR to elicit T cell immune response include or exclude but are not limited to IE-1 peptide 316-324 (VLEETSVML, SEQ ID NO:31). IE-1 is a HCMV protein and an antigen expressed by glioblastoma cells. In some embodiments, the epitopes include variants comprising 1, 2 or 3 conservative substitutions.

The term “self-cleaving peptide” includes but is not limited to the P2A sequence (RAKRSGSGATNFSLLKQAGDVEENPGP, Seq ID NO:51) that is located between two proteins and can be self-cleaved to separate the two proteins. Ryan, M. D., King, A. M. & Thomas, G. P. Cleavage of foot-and-mouth disease virus polyprotein is mediated by residues located within a 19 amino acid sequence. J. general virology 72 (Pt 11), 2727-2732, doi:10.1099/0022-1317-72-11-2727 (1991).

As used herein, the term “stimulation”, refers to a primary response induced by ligation of a cell surface moiety. For example, in the context of receptors, such stimulation entails the ligation of a receptor and a subsequent signal transduction event. With respect to stimulation of a T-cell, such stimulation refers to the ligation of a T-cell surface moiety that in one embodiment subsequently induces a signal transduction event, which can include or exclude binding the TCR/CD3 complex. Further, the stimulation event may activate a cell and upregulate or downregulate expression or secretion of a molecule, which can include or exclude downregulation of TGF-β. Thus, ligation of cell surface moieties, even in the absence of a direct signal transduction event, may result in the reorganization of cytoskeletal structures, or in the coalescing of cell surface moieties, each of which could serve to enhance, modify, or alter subsequent cell responses.

As used herein, the term “vector” includes but is not limited to pMSGV, pMSCV, pFU3W, or any other vector which functions as a carrier of the inserted DNA into live cells.

In yet another embodiment of the present invention, vectors for the insertion of cDNA encoding TCR alpha chain and/or TCR beta chain are provided. In some embodiments the translational product of the vector includes at least one alpha chain variable region combined with a least one alpha constant region and/or at least one beta chain variable region combined with at least one beta constant region, which are linked by a self-cleaving peptide. The vectors serve for the delivery of the insertion delivery into naïve T cells by viral transduction.

The term “viral transduction” as used herein, encompasses a procedure that produces recombinant viruses including but not limiting to retroviruses, lentiviruses, adeno-associated viruses, or other suitable viruses in host cells and uses these recombinant viruses containing the gene encoding the TCR in their genome to infect, transfect, or transduce target cells. The gene encoding the TCR is integrated to the genome of target cells and is stably expressed and replicated within proliferated cells. The term “target cell” includes but is not limited to CD4+ T cells, CD8+ T cells, tumor cells etc.

In one embodiment, the invention also provides host cells transfected or transduced with a vector comprising DNA encoding the TCR regions or chains, according to any preceding aspect or any aspect or embodiment disclosed herein, for example, the TCR alpha chain variable region combined with an alpha constant region and a beta chain variable region combined with a beta constant region, which are linked by P2A sequence (SEQ ID NO:51) for viral production and TCR delivery into naïve T cells.

In one embodiment, the TCRs are chimeric TCRs comprising a TCR variable region fused to a modified or non-human constant region. In some embodiments the chimeric TCR comprises a cancer antigen specific TCR variable region of any of the embodiments disclosed herein, fused to a non-human, e.g., murine TCR constant region. For example, the chimeric TCR may comprise a variable region that may include or exclude a CT83 TCR variable region, a NY-ESO-1 TCR variable region, a pp65 TCR variable region, or a IE-1 TCR fused to a non-human, e.g., murine TCR constant region. Among other features, the chimeric TCR reduces mispairing between the chimeric TCR and the endogenous TCR of the transduced T-cell. For example, a chimeric CT83 TCR(MC) reduce mispairing between the chimeric CT83 TCR(MC) and the endogenous TCR(HC) in the transduced cell. For example, a chimeric CT83 TCR reduces mispairing between the chimeric CT83 TCR(MC) and endogenous TCR(HC), or a chimeric NY-ESO-1 TCR reduces mispairing between the chimeric NY-ESO-1 (MC) and endogenous TCR(HC) or reduces mispairing. As another example, a chimeric pp65 TCR reduces mispairing between the chimeric pp65 (MC) and endogenous TCR(HC) or reduces mispairing. And as another example, a chimeric IE-1 TCR reduces mispairing between the chimeric IE-1 (MC) and endogenous TCR(HC) or reduces mispairing.

The term “host cell” includes but is not limited to cells from the PG-13 cell line, Phoenix-Eco cell line, Phoenix-Ampho cell line, 293GP cell line, or other suitable cell line which can intracellularly assemble the viral genome, package viruses with the capsule proteins, and secret mature viruses extracellularly.

In yet another embodiment of the present invention, the sequences of shRNAs or antisense RNAs or DNAs that specifically knock down metabolic genes to enhance the antitumor activity of TCR or CAR-based therapy in vivo are provided in the form of a stem of 21 base pairs. The TCR may be engineered together with the shRNAs by knocking down target genes to improve the T cell trafficking and persistence in vivo and enhance the antitumor activity. The term “knock down” or “ ” as referred to herein means to reduce expression of a gene, for example, by causing degradation of mRNA or by blocking RNA expression to decrease the protein expression of target genes. The term “metabolic gene” as referred to herein includes but is not limited to PD1, VHL and PPP2R2D.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

The present invention encompasses CD4+ or CD8+ T lymphocytes immunologically recognizing a tumor antigen with the restriction of one HLA class II or class I molecules. The present invention further encompasses at least one T cell receptor derived from the CD4+ T lymphocytes or CD8+ T lymphocytes mentioned above. The T cell receptors are capable of being delivered into naïve CD4+ or CD8+ T lymphocytes that have no immune response to the tumor antigens mentioned above, and changing the function of those CD4+ or CD8+ T lymphocytes to recognize and react with the tumor antigen mentioned above specifically. This reaction between the tumor antigens and the transduced T cell receptor causes the T lymphocytes to respond against, and assist in preventing, eliminating or reducing the human cancers with the tumor antigens mentioned above.

### B. Method of TCR Identification

### Immunoassays and Fluorochromes

The steps of various useful immunodetection methods have been described in the scientific literature, which can include or exclude, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (which can include or exclude the disclosed biomarkers) with an antibody to the molecule of interest or contacting an antibody to a molecule of interest (which can include or exclude antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, which can include or exclude a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (which can include or exclude the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, which can include or exclude any radioactive, fluorescent, biological or enzymatic tags or any other known label.

As used herein, a label can include a fluorescent dye, a member of a binding pair, which can include or exclude biotin/streptavidin, a metal (e.g., gold), or an epitope tag that can specifically interact with a molecule that can be detected, which can include or exclude by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the application as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 3SO™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson-; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow SGF; GeneBlazer; (CCF2); GFP (S65T); GFP red shifted (rsGFP); GFP wild type′ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; Sybr Green; Thiazole orange (interchelating dyes); semiconductor nanoparticles which can include or exclude quantum dots; or caged fluorophore (which can be activated with light or other electromagnetic energy source), or a combination thereof.

A modifier unit which can include or exclude a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the apset include, but are not limited to, Tc-99m, Re-186, Ga-68, Re-188, Y-90, Sm-153, Bi-212, Cu-67, Cu-64, and Cu-62. Radiolabeling techniques which can include or exclude these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques which can include or exclude positron emission tomography (PET) or single photon emission computerized tomography (SPECT).

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety which can include or exclude an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, which can include or exclude a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, which can include or exclude the biotin/avidin pair. In this mode, the detecting antibody or detecting molecule should include the other member of the pair.

Other modes of indirect labeling include the detection of primary immune complexes by a two-step approach. For example, a molecule (which can be referred to as a first binding agent), which can include or exclude an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-genarating molecule or moiety, allowing detection of the tertiary immune complexes thus formed. This system can provide for signal amplification.

Immunoassays that involve the detection of as substance, which can include or exclude a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, which can include or exclude size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

Provided that the concentrations are sufficient, the molecular complexes ([Ab-Ag]n) generated by antibody-antigen interaction are visible to the naked eye, but smaller amounts may also be detected and measured due to their ability to scatter a beam of light. The formation of complexes indicates that both reactants are present, and in immunoprecipitation assays a constant concentration of a reagent antibody is used to measure specific antigen ([Ab-Ag]n), and reagent antigens are used to detect specific antibody ([Ab-Ag]n). If the reagent species is previously coated onto cells (as in hemagglutination assay) or very small particles (as in latex agglutination assay), “clumping” of the coated particles is visible at much lower concentrations. A variety of assays based on these elementary principles are in common use, including Ouchterlony immunodiffusion assay, rocket immunoelectrophoresis, and immunoturbidometric and nephelometric assays. The main limitations of which can include or excludesays are restricted sensitivity (lower detection limits) in comparison to assays employing labels and, in some cases, the fact that very high concentrations of analyte can actually inhibit complex formation, necessitating safeguards that make the procedures more complex. Some of these Group 1 assays date right back to the discovery of antibodies and none of them have an actual “label” (e.g. Ag-enz). Other kinds of immunoassays that are label free depend on immunosensors, and a variety of instruments that can directly detect antibody-antigen interactions are now commercially available. Most depend on generating an evanescent wave on a sensor surface with immobilized ligand, which allows continuous monitoring of binding to the ligand. Immunosensors allow the easy investigation of kinetic interactions and, with the advent of lower-cost specialized instruments, may in the future find wide application in immunoanalysis.

The use of immunoassays to detect a specific protein can involve the separation of the proteins by electrophoresis. Electrophoresis is the migration of charged molecules in solution in response to an electric field. Their rate of migration depends on the strength of the field; on the net charge, size and shape of the molecules and also on the ionic strength, viscosity and temperature of the medium in which the molecules are moving. As an analytical tool, electrophoresis is simple, rapid and highly sensitive. It is used analytically to study the properties of a single charged species, and as a separation technique.

Generally the sample is run in a support matrix which can include or exclude paper, cellulose acetate, starch gel, agarose or polyacrylamide gel. The matrix inhibits convective mixing caused by heating and provides a record of the electrophoretic run: at the end of the run, the matrix can be stained and used for scanning, autoradiography or storage. In addition, the most commonly used support matrices—agarose and polyacrylamide—provide a means of separating molecules by size, in that they are porous gels. A porous gel may act as a sieve by retarding, or in some cases completely obstructing, the movement of large macromolecules while allowing smaller molecules to migrate freely. Because dilute agarose gels are generally more rigid and easy to handle than polyacrylamide of the same concentration, agarose is used to separate larger macromolecules which can include or exclude nucleic acids, large proteins and protein complexes. Polyacrylamide, which is easy to handle and to make at higher concentrations, is used to separate most proteins and small oligonucleotides that require a small gel pore size for retardation.

Proteins are amphoteric compounds; their net charge therefore is determined by the pH of the medium in which they are suspended. In a solution with a pH above its isoelectric point, a protein has a net negative charge and migrates towards the anode in an electrical field. Below its isoelectric point, the protein is positively charged and migrates towards the cathode. The net charge carried by a protein is in addition independent of its size—i.e., the charge carried per unit mass (or length, given proteins and nucleic acids are linear macromolecules) of molecule differs from protein to protein. At a given pH therefore, and under non-denaturing conditions, the electrophoretic separation of proteins is determined by both size and charge of the molecules.

Sodium dodecyl sulphate (SDS) is an anionic detergent which denatures proteins by “wrapping around” the polypeptide backbone—and SDS binds to proteins fairly specifically in a mass ratio of 1.4:1. In so doing, SDS confers a negative charge to the polypeptide in proportion to its length. Further, it is usually necessary to reduce disulfide bridges in proteins (denature) before they adopt the random-coil configuration necessary for separation by size; this is done with 2-mercaptoethanol or dithiothreitol (DTT). In denaturing SDS-PAGE separations therefore, migration is determined not by intrinsic electrical charge of the polypeptide, but by molecular weight.

Determination of molecular weight is done by SDS-PAGE of proteins of known molecular weight along with the protein to be characterized. A linear relationship exists between the logarithm of the molecular weight of an SDS-denatured polypeptide, or native nucleic acid, and its Rf. The Rf is calculated as the ratio of the distance migrated by the molecule to that migrated by a marker dye-front. A simple way of determining relative molecular weight by electrophoresis (Mr) is to plot a standard curve of distance migrated vs. log 10 MW for known samples, and read off the logMr of the sample after measuring distance migrated on the same gel.

In two-dimensional electrophoresis, proteins are fractionated first on the basis of one physical property, and, in a second step, on the basis of another. For example, isoelectric focusing can be used for the first dimension, conveniently carried out in a tube gel, and SDS electrophoresis in a slab gel can be used for the second dimension. One example of a procedure is that of O'Farrell, P. H., High Resolution Two-dimensional Electrophoresis of Proteins, J. Biol. Chem. 250:4007-4021 (1975), herein incorporated by reference in its entirety for its teaching regarding two-dimensional electrophoresis methods. Other examples include but are not limited to, those found in Anderson, L and Anderson, NG, High resolution two-dimensional electrophoresis of human plasma proteins, Proc. Natl. Acad. Sci. 74:5421-5425 (1977), Ornstein, L., Disc electrophoresis, L. Ann. N.Y. Acad. Sci. 121:321349 (1964), each of which is herein incorporated by reference in its entirety for teachings regarding electrophoresis methods. Laemmli, U.K., Cleavage of structural proteins during the assembly of the head of bacteriophage T4, Nature 227:680 (1970), which is herein incorporated by reference in its entirety for teachings regarding electrophoresis methods, discloses a discontinuous system for resolving proteins denatured with SDS. The leading ion in the Laemmli buffer system is chloride, and the trailing ion is glycine. Accordingly, the resolving gel and the stacking gel are made up in Tris-HCl buffers (of different concentration and pH), while the tank buffer is Tris-glycine. All buffers contain 0.1% SDS.

One example of an immunoassay that uses electrophoresis that is contemplated in the current methods is Western blot analysis. Western blotting or immunoblotting allows the determination of the molecular mass of a protein and the measurement of relative amounts of the protein present in different samples. Detection methods include chemiluminescence and chromogenic detection. Standard methods for Western blot analysis can be found in, for example, D. M. Bollag et al., Protein Methods (2d edition 1996) and E. Harlow & D. Lane, Antibodies, a Laboratory Manual (1988), U.S. Pat. No. 4,452,901, each of which is herein incorporated by reference in their entirety for teachings regarding Western blot methods. Generally, proteins are separated by gel electrophoresis, usually SDS-PAGE. The proteins are transferred to a sheet of special blotting paper, e.g., nitrocellulose, though other types of paper, or membranes, can be used. The proteins retain the same pattern of separation they had on the gel. The blot is incubated with a generic protein (which can include or exclude milk proteins) to bind to any remaining sticky places on the nitrocellulose. An antibody is then added to the solution which is able to bind to its specific protein.

The attachment of specific antibodies to specific immobilized antigens can be readily visualized by indirect enzyme immunoassay techniques, usually using a chromogenic substrate (e.g., alkaline phosphatase or horseradish peroxidase) or chemiluminescent substrates. Other possibilities for probing include the use of fluorescent or radioisotope labels (e.g., fluorescein, ¹²⁵I). Probes for the detection of antibody binding can be conjugated anti-immunoglobulins, conjugated staphylococcal Protein A (binds IgG), or probes to biotinylated primary antibodies (e.g., conjugated avidin/streptavidin).

The power of the technique lies in the simultaneous detection of a specific protein by means of its antigenicity, and its molecular mass. Proteins are first separated by mass in the SDS-PAGE, then specifically detected in the immunoassay step. Thus, protein standards (ladders) can be run simultaneously in order to approximate molecular mass of the protein of interest in a heterogeneous sample.

The gel shift assay or electrophoretic mobility shift assay (EMSA) can be used to detect the interactions between DNA binding proteins and their cognate DNA recognition sequences, in both a qualitative and quantitative manner. Exemplary techniques are described in Ornstein L., Disc electrophoresis-I: Background and theory, Ann. NY Acad. Sci. 121:321-349 (1964), and Matsudiara, PT and DR Burgess, SDS microslab linear gradient polyacrylamide gel electrophoresis, Anal. Biochem. 87:386-396 (1987), each of which is herein incorporated by reference in its entirety for teachings regarding gel-shift assays.

In a general gel-shift assay, purified proteins or crude cell extracts can be incubated with a labeled (e.g., ³²P-radiolabeled) DNA or RNA probe, followed by separation of the complexes from the free probe through a nondenaturing polyacrylamide gel. The complexes migrate more slowly through the gel than unbound probe. Depending on the activity of the binding protein, a labeled probe can be either double-stranded or single-stranded. For the detection of DNA binding proteins which can include or exclude transcription factors, either purified or partially purified proteins, or nuclear cell extracts can be used. For detection of RNA binding proteins, either purified or partially purified proteins, or nuclear or cytoplasmic cell extracts can be used. The specificity of the DNA or RNA binding protein for the putative binding site is established by competition experiments using DNA or RNA fragments or oligonucleotides containing a binding site for the protein of interest, or other unrelated sequence. The differences in the nature and intensity of the complex formed in the presence of specific and nonspecific competitor allows identification of specific interactions.

Gel shift methods can include using, for example, colloidal forms of COOMASSIE (Imperial Chemicals Industries, Ltd) blue stain to detect proteins in gels which can include or exclude polyacrylamide electrophoresis gels. Such methods are described, for example, in Neuhoff et al., Electrophoresis 6:427-448 (1985), and Neuhoff et al., Electrophoresis 9:255-262 (1988), each of which is herein incorporated by reference in its entirety for teachings regarding gel shift methods. In addition to the conventional protein assay methods referenced above, a combination cleaning and protein staining composition is described in U.S. Pat. No. 5,424,000, herein incorporated by reference in its entirety for its teaching regarding gel shift methods. The solutions can include phosphoric, sulfuric, and nitric acids, and Acid Violet dye.

Radioimmune Precipitation Assay (RIPA) is a sensitive assay using radiolabeled antigens to detect specific antibodies in serum. The antigens are allowed to react with the serum and then precipitated using a special reagent which can include or exclude, for example, protein A sepharose beads. The bound radiolabeled immunoprecipitate is then commonly analyzed by gel electrophoresis. Radioimmunoprecipitation assay (RIPA) is often used as a confirmatory test for diagnosing the presence of HIV antibodies. RIPA is also referred to in the art as Farr Assay, Precipitin Assay, Radioimmune Precipitin Assay; Radioimmunoprecipitation Analysis; Radioimmunoprecipitation Analysis, and Radioimmunoprecipitation Analysis.

While the above immunoassays that utilize electrophoresis to separate and detect the specific proteins of interest allow for evaluation of protein size, they are not very sensitive for evaluating protein concentration. However, also contemplated are immunoassays wherein the protein or antibody specific for the protein is bound to a solid support (e.g., tube, well, bead, or cell) to capture the antibody or protein of interest, respectively, from a sample, combined with a method of detecting the protein or antibody specific for the protein on the support. Examples of such immunoassays include Radioimmunoassay (RIA), Enzyme-Linked Immunosorbent Assay (ELISA), Flow cytometry, protein array, multiplexed bead assay, and magnetic capture.

Radioimmunoassay (RIA) is a classic quantitative assay for detection of antigen-antibody reactions using a radioactively labeled substance (radioligand), either directly or indirectly, to measure the binding of the unlabeled substance to a specific antibody or other receptor system. Radioimmunoassay is used, for example, to test hormone levels in the blood without the need to use a bioassay. Non-immunogenic substances (e.g., haptens) can also be measured if coupled to larger carrier proteins (e.g., bovine gamma-globulin or human serum albumin) capable of inducing antibody formation. RIA involves mixing a radioactive antigen (because of the ease with which iodine atoms can be introduced into tyrosine residues in a protein, the radioactive isotopes ¹²⁵I or ¹³¹I are often used) with antibody to that antigen. The antibody is generally linked to a solid support, which can include or exclude a tube or beads. Unlabeled or “cold” antigen is then adding in known quantities and measuring the amount of labeled antigen displaced. Initially, the radioactive antigen is bound to the antibodies. When cold antigen is added, the two compete for antibody binding sites—and at higher concentrations of cold antigen, more binds to the antibody, displacing the radioactive variant. The bound antigens are separated from the unbound ones in solution and the radioactivity of each used to plot a binding curve. The technique is both extremely sensitive, and specific.

Enzyme-Linked Immunosorbent Assay (ELISA), or more generically termed EIA (Enzyme ImmunoAssay), is an immunoassay that can detect an antibody specific for a protein. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase.

Variations of ELISA techniques are known to those of skill in the art. In one variation, antibodies that can bind to proteins can be immobilized onto a selected surface exhibiting protein affinity, which can include or exclude a well in a polystyrene microtiter plate. Then, a test composition suspected of containing a marker antigen can be added to the wells. After binding and washing to remove non-specifically bound immunocomplexes, the bound antigen can be detected. Detection can be achieved by the addition of a second antibody specific for the target protein, which is linked to a detectable label. This type of ELISA is a simple “sandwich ELISA.” Detection also can be achieved by the addition of a second antibody, followed by the addition of a third antibody that has binding affinity for the second antibody, with the third antibody being linked to a detectable label.

Another variation is a competition ELISA. In competition ELISA's, test samples compete for binding with known amounts of labeled antigens or antibodies. The amount of reactive species in the sample can be determined by mixing the sample with the known labeled species before or during incubation with coated wells. The presence of reactive species in the sample acts to reduce the amount of labeled species available for binding to the well and thus reduces the ultimate signal.

Regardless of the format employed, ELISAs have certain features in common, which can include or exclude coating, incubating or binding, washing to remove non-specifically bound species, and detecting the bound immunecomplexes. Antigen or antibodies can be linked to a solid support, which can include or exclude in the form of plate, beads, dipstick, membrane or column matrix, and the sample to be analyzed applied to the immobilized antigen or antibody. In coating a plate with either antigen or antibody, one will generally incubate the wells of the plate with a solution of the antigen or antibody, either overnight or for a specified period of hours. The wells of the plate can then be washed to remove incompletely adsorbed material. Any remaining available surfaces of the wells can then be “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means rather than a direct procedure can also be used. Thus, after binding of a protein or antibody to the well, coating with a non-reactive material to reduce background, and washing to remove unbound material, the immobilizing surface is contacted with the control clinical or biological sample to be tested under conditions effective to allow immunecomplex (antigen/antibody) formation. Detection of the immunecomplex then requires a labeled secondary binding agent or a secondary binding agent in conjunction with a labeled third binding agent.

Enzyme-Linked Immunospot Assay (ELISpot) is an immunoassay that can detect an antibody specific for a protein or antigen. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase. In this assay a nitrocellulose microtiter plate is coated with antigen. The test sample is exposed to the antigen and then reacted similarly to an ELISA assay. Detection differs from a traditional ELISA in that detection is determined by the enumeration of spots on the nitrocellulose plate. The presence of a spot indicates that the sample reacted to the antigen. The spots can be counted and the number of cells in the sample specific for the antigen determined.

“Under conditions effective to allow immunecomplex (antigen/antibody) formation” means that the conditions include diluting the antigens and antibodies with solutions which can include or exclude BSA, bovine gamma globulin (BGG) and phosphate buffered saline (PBS)/Tween so as to reduce non-specific binding and to promote a reasonable signal to noise ratio.

The suitable conditions also mean that the incubation is at a temperature and for a period of time sufficient to allow effective binding. Incubation steps can typically be from about 1 minute to twelve hours, at temperatures of about 20° to 30° C., or can be incubated overnight at about 0° C. to about 10° C.

Following all incubation steps in an ELISA, the contacted surface can be washed so as to remove non-complexed material. A washing procedure can include washing with a solution which can include or exclude PBS/Tween or borate buffer. Following the formation of specific immunecomplexes between the test sample and the originally bound material, and subsequent washing, the occurrence of even minute amounts of immunecomplexes can be determined.

To provide a detecting means, the second or third antibody can have an associated label to allow detection, as described above. This can be an enzyme that can generate color development upon incubating with an appropriate chromogenic substrate. Thus, for example, one can contact and incubate the first or second immunecomplex with a labeled antibody for a period of time and under conditions that favor the development of further immunecomplex formation (e.g., incubation for 2 hours at room temperature in a PBS-containing solution which can include or exclude PBS-Tween).

After incubation with the labeled antibody, and subsequent to washing to remove unbound material, the amount of label can be quantified, e.g., by incubation with a chromogenic substrate which can include or exclude urea and bromocresol purple or 2,2′-azido-di-(3-ethyl-benzthiazoline-6-sulfonic acid [ABTS] and H₂O₂, in the case of peroxidase as the enzyme label. Quantitation can then be achieved by measuring the degree of color generation, e.g., using a visible spectra spectrophotometer.

Protein arrays are solid-phase ligand binding assay systems using immobilized proteins on surfaces which include glass, membranes, microtiter wells, mass spectrometer plates, and beads or other particles. The assays are highly parallel (multiplexed) and often miniaturized (microarrays, protein chips). Their advantages include being rapid and automatable, capable of high sensitivity, economical on reagents, and giving an abundance of data for a single experiment. Bioinformatics support is important; the data handling demands sophisticated software and data comparison analysis. However, the software can be adapted from that used for DNA arrays, as can much of the hardware and detection systems.

One of the chief formats is the capture array, in which ligand-binding reagents, which are usually antibodies but can also be alternative protein scaffolds, peptides or nucleic acid aptamers, are used to detect target molecules in mixtures which can include or exclude plasma or tissue extracts. In diagnostics, capture arrays can be used to carry out multiple immunoassays in parallel, both testing for several analytes in individual sera for example and testing many serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens which can include or exclude protein-protein, protein-DNA, protein-drug, receptor-ligand, enzyme-substrate, etc. The capture reagents themselves are selected and screened against many proteins, which can also be done in a multiplex array format against multiple protein targets.

For construction of arrays, sources of proteins include cell-based expression systems for recombinant proteins, purification from natural sources, production in vitro by cell-free translation systems, and synthetic methods for peptides. Many of these methods can be automated for high throughput production. For capture arrays and protein function analysis, it is important that proteins should be correctly folded and functional; this is not always the case, e.g., where recombinant proteins are extracted from bacteria under denaturing conditions. Nevertheless, arrays of denatured proteins are useful in screening antibodies for cross-reactivity, identifying autoantibodies and selecting ligand binding proteins.

Protein arrays have been designed as a miniaturization of familiar immunoassay methods which can include or exclude ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics (Gyros, Monmouth Junction, N.J.) and specialised chip designs, which can include or exclude engineered microchannels in a plate (e.g., The Living Chip™, Biotrove, Woburn, Mass.) and tiny 3D posts on a silicon surface (Zyomyx, Hayward Calif.). Particles in suspension can also be used as the basis of arrays, providing they are coded for identification; systems include colour coding for microbeads (Luminex, Austin, Tex.; Bio-Rad Laboratories) and semiconductor nanocrystals (e.g., QDots™, Quantum Dot, Hayward, Calif.), and barcoding for beads (UltraPlex™, SmartBead Technologies Ltd, Babraham, Cambridge, UK) and multimetal microrods (e.g., Nanobarcodes™ particles, Nanoplex Technologies, Mountain View, Calif.). Beads can also be assembled into planar arrays on semiconductor chips (LEAPS technology, BioArray Solutions, Warren, N.J.).

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents. In the Versalinx™ system (Prolinx, Bothell, Wash.) reversible covalent coupling is achieved by interaction between the protein derivatised with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures which can include or exclude HydroGel™ (PerkinElmer, Wellesley, Mass.), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilized on a surface which can include or exclude titanium dioxide (Zyomyx) or tantalum pentoxide (Zeptosens, Witterswil, Switzerland).

Array fabrication methods include robotic contact printing, ink-jetting, piezoelectric spotting and photolithography. A number of commercial arrayers are available [e.g., Packard Biosciences] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85sq microns, equivalent to 25 million spots per sq cm, at the limit for optical detection; their readout methods are fluorescence and atomic force microscopy (AFM).

Fluorescence labeling and detection methods are widely used. The same instrumentation as used for reading DNA microarrays is applicable to protein arrays. For differential display, capture (e.g., antibody) arrays can be probed with fluorescently labeled proteins from two different cell states, in which cell lysates are directly conjugated with different fluorophores (e.g., Cy-3, Cy-5) and mixed, such that the color acts as a readout for changes in target abundance. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Intrinsic Bioprobes, Tempe, Ariz.), rolling circle DNA amplification (Molecular Staging, New Haven Conn.), mass spectrometry (Intrinsic Bioprobes; Ciphergen, Fremont, Calif.), resonance light scattering (Genicon Sciences, San Diego, Calif.) and atomic force microscopy [BioForce Laboratories].

Capture arrays form the basis of diagnostic chips and arrays for expression profiling. They employ high affinity capture reagents, which can include or exclude conventional antibodies, single domains, engineered scaffolds, peptides or nucleic acid aptamers, to bind and detect specific target ligands in high throughput manner.

Antibody arrays have the required properties of specificity and acceptable background, and some are available commercially (BD Biosciences, San Jose, Calif.; Clontech, Mountain View, Calif.; BioRad; Sigma, St. Louis, Mo.). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; BioInvent, Lund, Sweden; Affitech, Walnut Creek, Calif.; Biosite, San Diego, Calif.). In addition to the conventional antibodies, Fab and scFv fragments, single V-domains from camelids or engineered human equivalents (Domantis, Waltham, Mass.) may also be useful in arrays.

The term “scaffold” refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staph. aureus protein A (Affibody, Bromma, Sweden), ‘Trinectins’ based on fibronectins (Phylos, Lexington, Mass.) and ‘Anticalins’ based on the lipocalin structure (Pieris Proteolab, Freising-Weihenstephan, Germany). These can be used on capture arrays in a similar fashion to antibodies and may have advantages of robustness and ease of production.

Nonprotein capture molecules, notably the single-stranded nucleic acid aptamers which bind protein ligands with high specificity and affinity, are also used in arrays (SomaLogic, Boulder, Colo.). Aptamers are selected from libraries of oligonucleotides by the Selex™ procedure and their interaction with protein can be enhanced by covalent attachment, through incorporation of brominated deoxyuridine and UV-activated crosslinking (photoaptamers). Photocrosslinking to ligand reduces the crossreactivity of aptamers due to the specific steric requirements. Aptamers have the advantages of ease of production by automated oligonucleotide synthesis and the stability and robustness of DNA; on photoaptamer arrays, universal fluorescent protein stains can be used to detect binding.

Protein analytes binding to antibody arrays may be detected directly or via a secondary antibody in a sandwich assay. Direct labelling is used for comparison of different samples with different colors. Where pairs of antibodies directed at the same protein ligand are available, sandwich immunoassays provide high specificity and sensitivity and are therefore the method of choice for low abundance proteins which can include or exclude cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately; serial dilution of the sample or use of antibodies of different affinities are solutions to this problem. Proteins of interest are frequently those in low concentration in body fluids and extracts, requiring detection in the pg range or lower, which can include or exclude cytokines or the low expression products in cells.

An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (ProteinPrint™, Aspira Biosystems, Burlingame, Calif.).

Another methodology which can be used diagnostically and in expression profiling is the ProteinChip® array (Ciphergen, Fremont, Calif.), in which solid phase chromatographic surfaces bind proteins with similar characteristics of charge or hydrophobicity from mixtures which can include or exclude plasma or tumour extracts, and SELDI-TOF mass spectrometry is used to detection the retained proteins.

Large-scale functional chips have been constructed by immobilizing large numbers of purified proteins and used to assay a wide range of biochemical functions, which can include or exclude protein interactions with other proteins, drug-target interactions, enzyme-substrates, etc. Generally they require an expression library, cloned into E. coli, yeast or similar from which the expressed proteins are then purified, e.g., via a His tag, and immobilized. Cell free protein transcription/translation is a viable alternative for synthesis of proteins which do not express well in bacterial or other in vivo systems.

For detecting protein-protein interactions, protein arrays can be in vitro alternatives to the cell-based yeast two-hybrid system and may be useful where the latter is deficient, which can include or exclude interactions involving secreted proteins or proteins with disulphide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilised on a microarray. Large-scale ‘proteome chips’ promise to be very useful in identification of functional interactions, drug screening, etc. (Proteometrix, Branford, Conn.).

As a two-dimensional display of individual elements, a protein array can be used to screen phage or ribosome display libraries, in order to select specific binding partners, including antibodies, synthetic scaffolds, peptides and aptamers. In this way, ‘library against library’ screening can be carried out. Screening of drug candidates in combinatorial chemical libraries against an array of protein targets identified from genome projects is another application of the approach.

A multiplexed bead assay, which can include or exclude, for example, the BD™ Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e., through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

Accordingly, in one aspect, disclosed herein are methods of identifying T cell receptors disclosed herein, wherein the T cell activity (which can include or exclude, for example, release of cytokines including, but not limited to IFN-γ, TGF-β, lymphotoxin-α, IL-2, IL-4, IL-10, IL-17, or IL-25) is measured by any immunodetection disclosed herein, for example, without limitation, by ELISA, ELISpot, Intracellular cytokine staining, or Chromium Release.

It is understood and herein contemplated that the disclosed T cell receptors (for example, without limitation, T cell receptors that bind cancer antigens that can include or exclude any of DP4-ESO-1 TCR, A2-CT83 TCR, A2-pp65-TCR and/or A2-IE-1-TCR) can be used to treat any cancer that express certain types of MHC molecules and antigens, including, but not limited to B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers, small cell lung cancer, non-small cell lung cancer, neuroblastoma, glioblastoma, ovarian cancer, pancreatic cancer, prostate cancer, skin cancers, melanoma, basal cell carcinoma, squamous cell carcinoma, liver cancer, squamous cell carcinomas of the mouth, throat, larynx, and lung, cervical cancer, cervical carcinoma, breast cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, AIDS-related lymphomas, or AIDS-related sarcomas. Therefore, in one aspect, also disclosed herein are methods of identifying TCR disclosed herein, wherein the cancer is selected from the group consisting of B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers, small cell lung cancer, non-small cell lung cancer, neuroblastoma, glioblastoma, ovarian cancer, pancreatic cancer, prostate cancer, skin cancers, melanoma, basal cell carcinoma, squamous cell carcinoma, liver cancer, squamous cell carcinomas of the mouth, throat, larynx, and lung, cervical cancer, cervical carcinoma, breast cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, AIDS-related lymphomas, or AIDS-related sarcomas.

### C. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular TCR is disclosed and discussed and a number of modifications that can be made to a number of molecules including the TCR are discussed, specifically contemplated is each and every combination and permutation of TCR and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

In one embodiment, the methods disclosed herein detect or identify TCRs that can be used in compositions for the treatment of cancer (either as a therapeutic treatment or prophylactic treatment) as well as in preparing TCR T cells that can be used to treat cancer. Thus, in one embodiment, also disclosed are TCR T cells engineered to express a receptor (which can include or exclude, for example, a T cell receptor) that can recognize antigens disclosed herein.

In one embodiment, this disclosure also feature methods to enhance CAR-T and TCR cell persistence by expression of chemokine receptor and shRNA KO in TCR or CAR constructs. In one embodiment, the TCR T cell specific for one antigen (for example, NY-ESO-1, CT83, pp65 and/or IE-1) disclosed herein can be further engineered to knockout or knockdown a negative signaling molecule, for example without limitation, programmed cell death protein (PD1), von Hippel-Lindau tumor suppressor (VHL), and/or protein phosphatase 2 regulatory subunit Bdelta (PPP2R2D) to enhance their function which can include or exclude cytotoxic activity and persistence or survival in vivo after adoptive transfer to a cancer patient.

In some embodiments the negative signaling molecules are, for example, indoleamine (2,3)-dioxygenase (IDO) (including isoforms IDO1 and IDO2), OX40, CTLA-4 (programmed cytotoxic T-lymphocyte antigen 4), PD-1 (programmed death 1), PD-L1 (programmed death ligand 1), PD-L2, lymphocyte activation gene 3 (LAG3), and B7 homolog 3 (B7-H3). In certain aspects, the negative signaling molecules are, for example, PD-1, VHL, PPP2R2D and epigenetic factors which can include or exclude JMJD3 and LSD1.

In one embodiment, this disclosure also features methods to enhance T cell trafficking into tumors and/or the location of tumor cells in vivo by forced expression of chemokine receptors. In some embodiments, expression of chemokine receptors is forced by fusing a CAR or TCR construct withi a chemokine. In some embodiments, the chemokine receptor is CCR5, CCR2 of CXCR3. In some embodiments, the chemokine receptor is CCR5.

It is understood and herein contemplated that once the sequence of a TCR is identified, the skilled artisan would have full knowledge of the nucleic acids that would encode said amino acid TCR and it would be well within the skill set of the skilled artisan to make said nucleic acid constructs. Thus, in one aspect, also disclosed herein are nucleic acids encoding a polypeptide for any TCR disclosed herein.

### Identity/Homology

It is understood that one way to define any known variants and derivatives or those that might arise, of the disclosed genes and proteins herein is through defining the variants and derivatives in terms of identity or homology and/or identify to specific known sequences. For example, Seq ID NO: 3 sets forth a particular sequence of a TCR alpha chain variable region. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology or identity to the stated sequence. Those of skill in the art readily understand how to determine the homology or identity of two proteins or nucleic acids, which can include or exclude genes. For example, the homology or identity can be calculated after aligning the two sequences so that the homology is at its highest level.

Another way of calculating homology or identity can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology or identity can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

### Nucleic Acids

There are a variety of molecules disclosed herein that are nucleic acid based, including for example the nucleic acids that encode, for example SEQ ID NO: 1, or any of the nucleic acids disclosed herein or fragments thereof, as well as various functional nucleic acids. In some embodiments, the nucleic acids included herein can include or exclude cDNA encoding TCR alpha chain and/or TCR beta chain. As used herein the term “cDNA” refers to a nucleic acid which joins exon-exon, or exon-only coding sequences. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment. In some embodiments the nucleotide analogs comprise one or more modifications to one or more base, sugar, or phosphate moieties in the nucleic acid as disclosed further herein.

### Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 2-methylcytosine (2-me-C),5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, which can include or exclude peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties which can include or exclude a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

### Primers and Probes

Disclosed are compositions including primers and probes, which are capable of interacting with the disclosed nucleic acids, which can include or exclude the tumor antigens, epitopes and TCRs disclosed herein. In certain embodiments the primers are used to support DNA amplification reactions. Typically, the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, which can include or exclude PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically, the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, which can include or exclude DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In certain embodiments this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

### Peptides

### Protein Variants

As discussed herein there are numerous variants of the TCRs that are known and herein contemplated. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, which can include or exclude those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Substitutions may include or exclude substitutions in one or more of the six TCR CDR regions. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2
Amino Acid Substitutions
Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations which can include or exclude, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology/identity to the stated sequence. Those of skill in the art readily understand how to determine the homology/identity of two proteins. For example, the homology/identity can be calculated after aligning the two sequences so that the homology/identiy is at its highest level.

Another way of calculating homolog/identity can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology/identity can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

The description of conservative mutations and homology/identity can be combined together in any combination, which can include or exclude embodiments that have at least 70% homology/identity to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site-specific way.

Molecules can be produced that resemble peptides, but which are not connected via a natural peptide linkage. For example, linkages for amino acids or amino acid analogs can include CH₂NH—, —CH₂S—, —CH₂—CH₂—CH═CH—(cis and trans), —COCH₂—CH(OH)CH₂—, and —CHH₂SO—(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, which can include or exclude b-alanine, g-aminobutyric acid, and the like.

Amino acid analogs and analogs and peptide analogs often have enhanced or desirable properties, which can include or exclude, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

### Pharmaceutical Carriers/Delivery of Pharmaceutical Products

In one aspect, disclosed herein are compositions comprising a therapeutically effective amount of one or more TCR T-cells; wherein TCR T cell has been engineered to express a receptor for one tumor antigen disclosed herein. In one aspect, the TCR T cell specific for one tumor antigen disclosed herein can be further engineered to knockout or knockdown programmed cell death protein (PD1), von Hippel-Lindau tumor suppressor (VHL), and/or protein phosphatase 2 regulatory subunit Bdelta (PPP2R2D) to enhance their function which can include or exclude cytotoxic activity and persistence or survival in vivo after adoptive transfer to a cancer patient.

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e. g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e. g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles which can include or exclude “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, which can include or exclude nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations which can include or exclude semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions which can include or exclude sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients which can include or exclude antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils which can include or exclude olive oil, and injectable organic esters which can include or exclude ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (which can include or exclude those based on Ringer's dextrose), and the like. Preservatives and other additives may also be present which can include or exclude, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids which can include or exclude hydrochloric acid, hydrobromic acid, perchloric acid, nitric acid, thiocyanic acid, sulfuric acid, and phosphoric acid, and organic acids which can include or exclude formic acid, acetic acid, propionic acid, glycolic acid, lactic acid, pyruvic acid, oxalic acid, malonic acid, succinic acid, maleic acid, and fumaric acid, or by reaction with an inorganic base which can include or exclude sodium hydroxide, ammonium hydroxide, potassium hydroxide, and organic bases which can include or exclude mono-, di-, trialkyl and aryl amines and substituted ethanolamines.

### Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, which can include or exclude unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above. It can generally be stated that a pharmaceutical composition comprising the subject engineered T cells, may be administered at a dosage of 10⁴ to 10⁷ engineered T cells/kg body weight, preferably 10⁵ to 10⁶ engineered T cells/kg body weight, including all integer values within those ranges. Engineered T cells compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

### D. Methods of Using the Compositions

### Method of Treating Cancer

The disclosed compositions can be used to treat any disease where uncontrolled cellular proliferation occurs which can include or exclude cancers. Accordingly, in one aspect, disclosed herein are methods of stimulating an immunological response against a cancer or treating, inhibiting, and/or preventing a cancer comprising administering to a subject a composition comprising a therapeutically effective amount of T cells engineered with antigen-specific TCRs disclosed herein (for example, they may include or exclude any of SEQ ID NO: 3, SEQ ID NO: 4, SEQ ID NO: 5, SEQ ID NO: 6, SEQ ID NO:20, SEQ ID NO:21, SEQ ID NO:22, SEQ ID NO:23, SEQ ID NO:24, SEQ ID NO:25, SEQ ID NO:27, SEQ ID NO:28, SEQ ID NO:29, SEQ ID NO:30, SEQ ID NO:32, or SEQ ID NO:33).

The term “therapeutically effective” refers to the amount of the composition used is of sufficient quantity to ameliorate one or more causes or symptoms of a disease or disorder. Such amelioration only requires a reduction or alteration, not necessarily elimination. The precise amount of the compositions of the present invention to be administered can be determined by a physician with consideration of individual differences in age, weight, tumor size, extent of infection or metastasis, and condition of the patient.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

A non-limiting list of different types of cancers that can be treated by the disclosed methods is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumors, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers which can include or exclude small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, and/or rectal cancers.

### E. Examples

### Example 1: Identification, Cloning, Construction and Application of DP4-ESO-1 TCR

### Generation of HLA-DP4-Restricted NY-ESO-1 Specific T Cells and Clones

In this invention, HLA-DP4 restricted TCRs were cloned from T cell clones that have specific recognition against the HLA-DP4 presented NY-ESO-1 peptide. To obtain peptide-reactive T cells, in vitro sensitization was carried out. NY-ESO-1₁₆₁₋₁₈₀, a peptide containing the HLA-DP4 restricted epitope, was synthesized with >95% purity. The peptide was pulsed on a HLA-DP4+ cell line, 1088EBV-B cells as APCs and cocultured with human PBMCs in 96-well plates for 21 days. During the procedure, the T cell population specific to NY-ESO-1₁₆₁₋₁₈₀ might be expanded under the pressure of continuous peptide stimulation, while non-specific T cells and other types of immune cells might be exhausted. After 21 days of stimulation, the T cells population in different wells were harvested for the further characterization.

The recognition of the stimulated T cells from different wells against NY-ESO-1₁₆₁₋₁₈₀ was firstly examined. T cells were cocultured with mock 1088EBV-B cells and the same APCs pulsed with the target peptide and then the activation of T cells was determined by the cytokines release in the supernatant. T cell populations from a few wells showed quite high activity against the peptide. These T cells populations were collected for depletion of CD8+ T cells. After CD8+ T cells were depleted, the CD4+ T cell line was designated DP4 ESO-reactive T cells. The T cell line was verified to recognize HLA-DP restricted NY-ESO-1 epitopes, but not HLA-DR restricted. Meanwhile the specific peptide recognition of DP4 ESO-1-reactive T cells was only blocked by antibodies against all the HLA class II molecules and HLA-DP molecules, indicating the recognition was HLA-DP restricted excluding other class I and class II molecules. The attempt to further identify the DP4 ESO-reactive T cell epitope was also performed. The shortest truncate which maintained high T cell recognition and activity was then limited to NY-ESO-1₁₅₇₋₁₇₀.

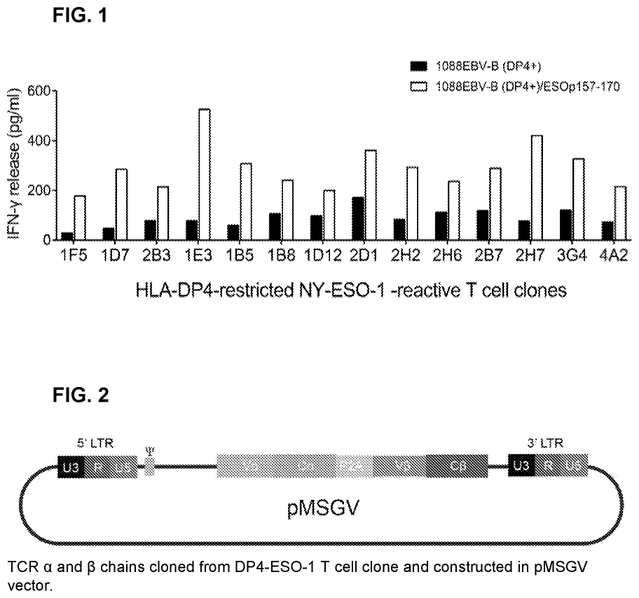

To perform the molecular cloning of DP4 restricted NY-ESO-1 TCR from DP4 ESO-reactive T cell line, single T cell clones were generated from the population. DP4 ESO-reactive T cell line was serially diluted and seeded in 96-well plates at the ratio of 0.3 cells/well. Single live T cells in the wells were cultured for expansion for 14 days. The expanded T cell clones were assayed with 1088EBV-B (HLA-DP4+) presented peptide NY-ESO-1₁₅₇₋₁₇₀ and the recognition of these clones against the epitope was determined by measurement of cytokine release. The assay result was partially shown in FIG. 1, demonstrating several DP4-ESO-1-reactive T cell clones reacted with the peptide NY-ESO-1₁₅₇₋₁₇₀ compared to mock APCs. These peptide-reactive T cell clones were cultured for expansion for another 14 days. However, quite a few DP4-ESO-1-reactive T cell clones survived after the expansion while a portion of T cells clones were exhausted and stopped growing, indicating the lifespan of these T cell clones differed. Surviving T cell clones were further used for TCR cloning.

### Molecular Cloning of DP4-ESO-1 TCR

With live T cell clones, mRNA was extracted from 1×10⁶ T cells. Reverse transcription was performed with mRNA to generate templates for the following three rounds of nested PCR. During each round of PCR, the Complementarity-determining region 3 (CDR3) of TCR alpha and beta chains were amplified separately with different primer sets containing primers targeting all types of TRAV or TRBV. After the recovery of PCR products from the 3rd round, the subtypes of TCR alpha and beta chain were identified by Sanger-sequencing the PCR products. Then the full length of TCR alpha and beta chains were amplified according to the identified TCR subtypes (TRAV34, TRBV30). The amplified full-length TCR (TRAV-TRAJ-TRAC, TRBV-TRBD-TRBJ-TRBC) were cloned into MSGV retroviral vector, which were linked by P2A sequence (FIG. 2).

### Transduction of DP4-TCR into Naïve T Cells for In Vitro and In Vivo Function

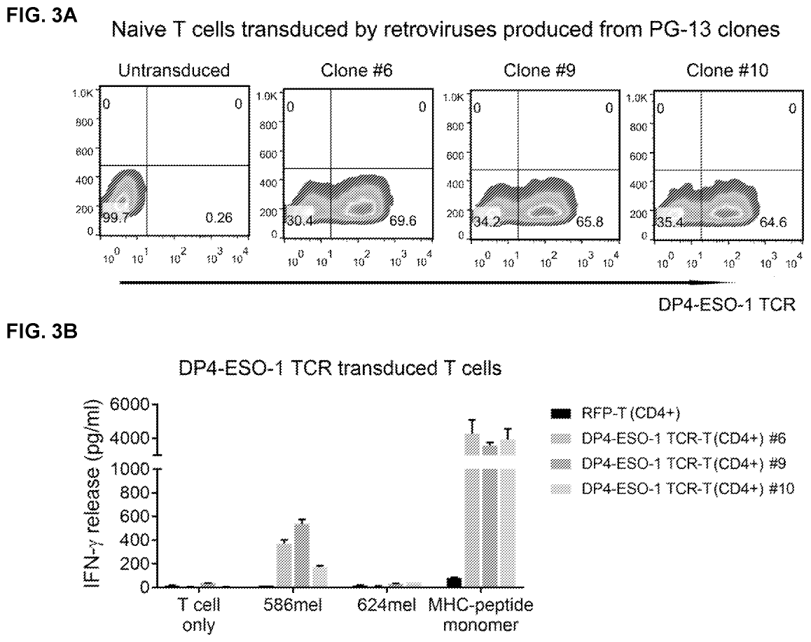

A two-step transduction strategy was applied to deliver TCR into naïve T cells. The TCR construct was transfected into Phoenix-Eco cell line with lipofectamine to generate primary viral supernatant. 48 h later, viral supernatant was collected and added to the culture medium of PG-13 cell line to infect them. After the infection, PG-13 cell line started to secret secondary viral supernatant. The viral supernatant from PG-13 cell line was coated onto 24-well non-tissue culture plates that were pre-coated with Retronectin. Naïve CD4+ T cells, which were bead-isolated from human PBMCs and pre-stimulated by CD3 antibody, were infected in the well by coated retroviruses. To improve the transduction efficiency to naïve T cells, the PG-13 cell line transduced with the TCR-encoding construct was cloned by limited dilution in 96-well plates. Single PG-13 cells were expanded in each well for 15 or more days to generate 100% TCR-encoding PG-13 cell lines. The transduction efficiency of naïve T cells from different PG-13 clones was determine by staining with TRBV30-specific antibody and flow cytometry, which reached to 60-70% on average (FIG. 3A). The activity of TCR-transduced CD4+ T cells was tested with 586mel, 624mel and DP4-ESO monomer, showing the specific recognition of the TCR (FIG. 3B).

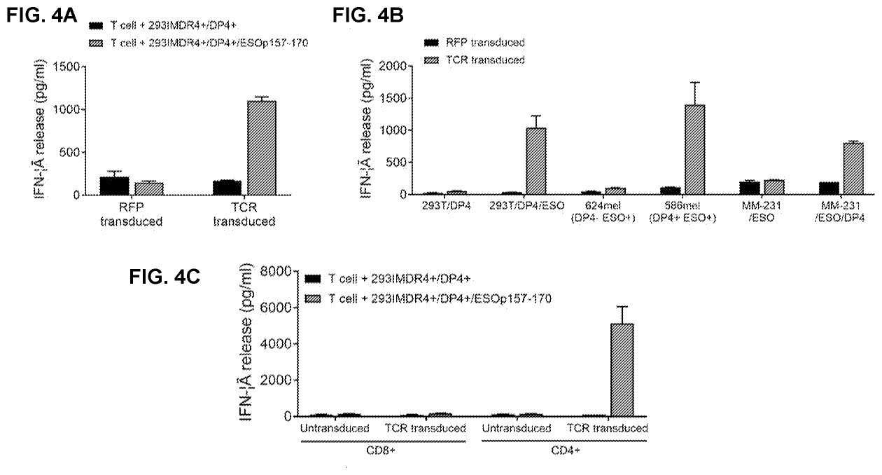

The activity of TCR-transduced T cells was further tested with a serial of assays to check whether they had similar function as DP4 ESO-reactive T cell line. Firstly, the transduced T cells showed specific recognition against the HLA-DP4 restricted epitope NY-ESO-1₁₅₇₋₁₇₀ (FIG. 4A). Secondly, the transduced T cells specifically recognized naturally HLA-DP4 processed NY-ESO-1, both when assayed with plasmids transfected in artificial APCs or HLA-DP4 positive or negative tumor cells (FIG. 4B). To confirm the cloned TCR functioned as a CD4+ TCR, bead-isolated CD8+ and CD4+ T cells were transduced respectively. The assay showed only CD4+ T cells transduced with this TCR were functional with the HLA-DP4 restricted epitope, which was consistent with the expectation (FIG. 4C). All the results confirmed the ability of the TCR-engineered CD4+ T cells to recognize HLA-DP4 presented NY-ESO-1 specifically. In one embodiment, the invention of this HLA-DP4 restricted NY-ESO-1 TCR serves as a new strategy for clinical response in cancer immunotherapy.

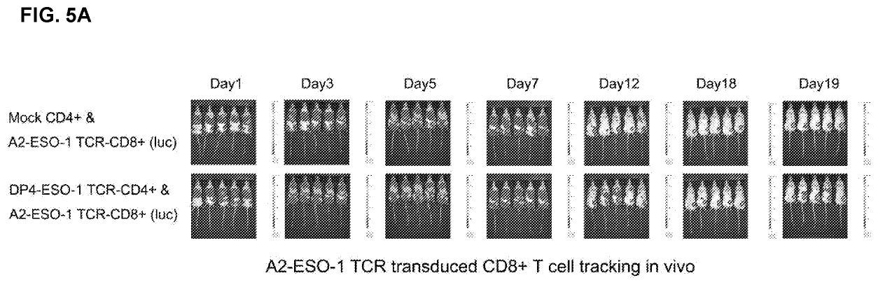

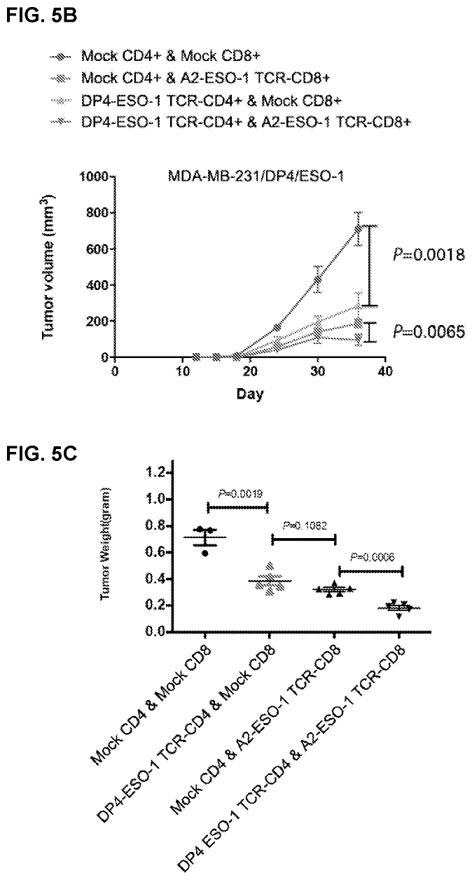

Humanized mice were used to assess the potency and safety of NY-ESO-1 TCRs due to the limitations of using transgenic mice. Humanized NSG (NOD SCID IL2γ−/−) mice were obtained and used for testing human cancer cell growth. MDA-MB-231/DP4/ESO tumor cells in 50 μl growth medium/matrigel (50%) were prepared and orthotopically injected into the 4th fat pad of female NSG mice (n=6 per group) on day 1. Following injection, it was observed that this human tumor line could grow in NSG mice. On day 5, tumor-bearing NSG mice were treated with 4 groups of human T cells (1. untransduced CD8+ and CD4+ T cells; 2. A2-ESO-1 TCR-CD8+ and untransduced CD4+ T cells; 3. untransduced CD8+ and DP4-ESO-1 TCR-CD4+ T cells; 4. A2-ESO-1 TCR-CD8+ and DP4-ESO-1 TCR-CD4+) at 2×10⁶ cells per mouse by i. v. injection, followed with 3 doses of IL-2 by i.p. injection to boost T cells proliferation. The tumor growth in each group was monitored every 3-5 days and the T-cell migration was tracked by luciferase transduced in CD8+ T cells. The results showed that injected A2-ESO-TCR-engineered CD8+ T cells gradually but significantly migrated to the tumor site after injection, indicating the specificity and safety of engineered T cells in vivo (FIG. 5A). A2-ESO-1 TCR-engineered CD8+ T cells also dramatically inhibited tumor growth as we expected (FIG. 5B, 5C). It was surprisingly found that DP4-ESO-1 TCR-CD4+ T cells exhibited significant suppression of tumor growth as well as A2-ESO-1 TCR-engineered CD8+ T cells (FIG. 5B, 5C), which indicated CD4+ cytolytic activity was activated in this case. Furthermore, the combination of A2-ESO-1 TCR-CD8+ T cells and DP4-ESO-1 TCR CD4+ T cells achieved the maximum inhibition on tumor growth when the mice were sacrificed (FIG. 5B, 5C), indicating the combined usage of CD8+ and CD4+ T cells transduced with TCRs targeting the same antigen was better than either transduced TCR-T cell type alone. It confirmed that CD4+ T cells provided efficient help to the antitumor activity of CD8+ T cells, providing surprising synergistic results.

Six- to eight-week-old female NSG mice were purchased from Jackson Laboratory or bred in the animal facility at Houston Methodist Research Institute. All procedures were approved by Houston Methodist Research Institute Animal Care and Use Committee (IACUC) and performed under lab standard protocol. MDA-MB-231/DP4/ESO tumor cells in 50 μl growth medium/matrigel (50%) were orthotopically injected into the 4th fat pad of female NSG mice (n=6 per group) on day 1. Tumor-bearing NSG mice were i. v. injected with 4 groups of human T cells (1. untransduced CD8+ and CD4+; 2. A2-ESO-1 TCR-CD8+ and untransduced CD4+; 3. untransduced CD8+ and DP4-ESO-1 TCR-CD4+; 4. A2-ESO-1 TCR-CD8+ and DP4-ESO-1 TCR-CD4+) at 2×10⁶ cells per mouse on day 5, followed with 3 doses of IL-2 by i.p. injection. The tumor growth was monitored every 3-5 days and the T-cell migration was tracked by luciferase transduced in CD8+ T cells.

### Example 2: Identification, Construction and Application of T Cells and A2-CT83 TCR-T Cells

### Identification of Novel HLA-A2 Restricted CT83 Epitopes Recognized by T Cells

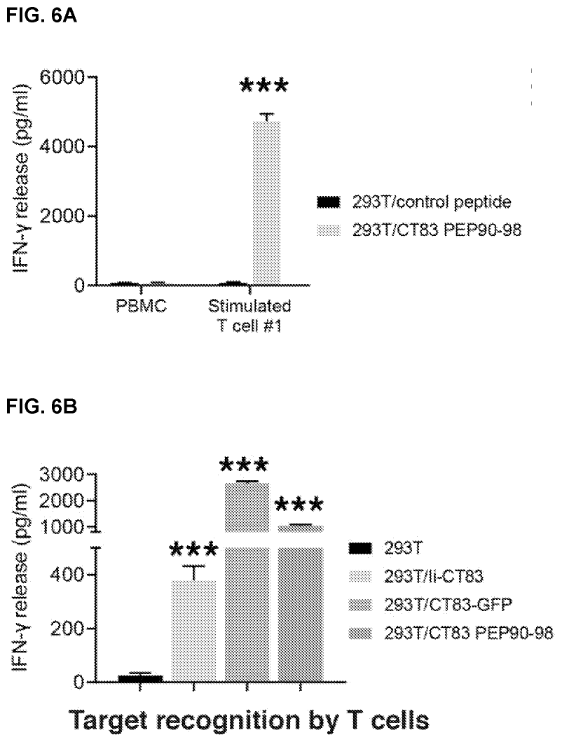

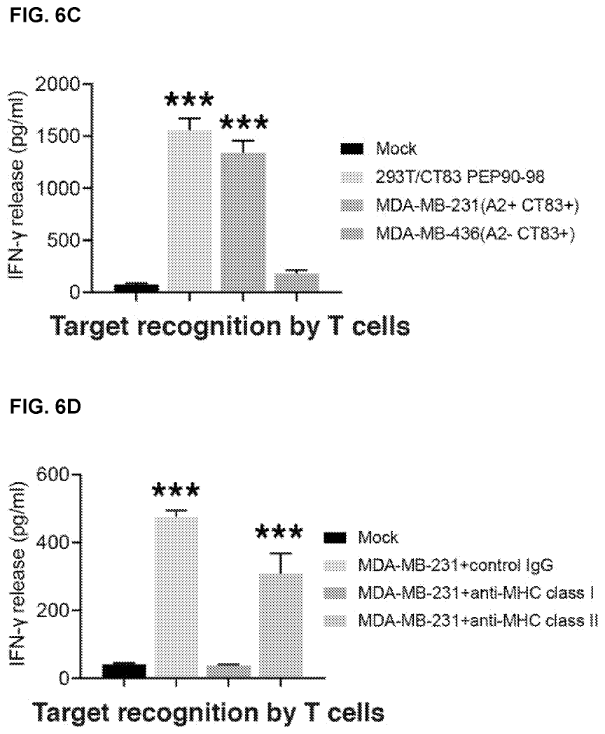

Because HLA-A2 is a predominant HLA class I molecule and highly expressed in approximately 50% of general human population, experiments were conducted to identify novel HLA-A2-restricted T cell epitopes from CT83. For this reason, a series of CT83 peptides were synthesized, containing potential HLA-A2 binging motifs (CT83 PEP66-74, PEP79-87 and PEP90-98). CD8+ T cells were isolated from PBMCs of HLA A2+ healthy donors, and stimulated with autologous dendritic cells (DCs) loaded with peptides for 10 days. T cell medium containing IL-7 and IL-15 (5 ng/ml each) were added every 2-3 days. For the second stimulation, autologous PBMCs were irradiated (60 Gy) and pulsed with 1 μg/ml peptide for 2-4 h. After washes, these irradiated peptide-pulsed PBMCs were added and incubated with the first stimulated T cells for 10 days. These T cells were fed with T cell medium containing IL-2 (30 IU/ml), IL-7 (5 ng/ml) and IL-15 (5 ng/ml) every 2-3 days. To test their specific recognition of CT83, in vitro stimulated T cells were incubated with HLA-A2⁺ HEK293T cells with or without CT83 peptide₉₀₋₉₈. The results showed that in vitro peptide stimulated T cells specifically recognized 293T/CT83 peptide (PEP90-98), but did not respond to 293T cells (FIG. 6A). T cells specific for CT83 PEP66-74 or CT83 PEP79-87 were not generated (data not shown). To determine whether these T cells recognize endogenously processed and presented CT83 PEP90-98 by HLA-A2, they were tested against 293T cells transfected with either invariant chain (Ii) fused CT83 or CT83-GFP (full-length CT83 and green fluorescent protein (GFP) linked with P2A sequence). 293T/control peptide and 293T/CT83 PEP90-98 served as negative and positive controls. The results showed that CT83-specific T cells recognized 293T/Ii-CT83 and 293T/CT83-GFP cells, as well as 293T/CT83 PEP90-98, but not 293T/control peptide (FIG. 6B). Next, it was determined whether these CT83-specific T cells could recognize breast cancer cells, and it was found that MDA-MB-231 cells (expressing CT83 and HLA-A2), but not MDA-MB-468 cells (expressing CT83, but not HLA-A2), were capable of stimulating CT83-specific T cells to secrete interferon-γ (IFN-γ) (FIG. 6C), suggesting that T cells recognize CT83 PEP90-98 presented by HLA-A2 molecules. To further validate this point, an antibody blocking experiment was performed and the results showed that T cell recognition of MDA-MB-231 cells could be completely blocked by anti-MHC-I, but not by anti-MHC-II or control antibody (FIG. 6D), indicating that these T cells are specific and capable of recognizing breast cancer cells naturally expressing CT83 and HLA-A2 molecules.

### Vaccination Using CT83 PEP90-98 Inhibits Breast Cancer Growth

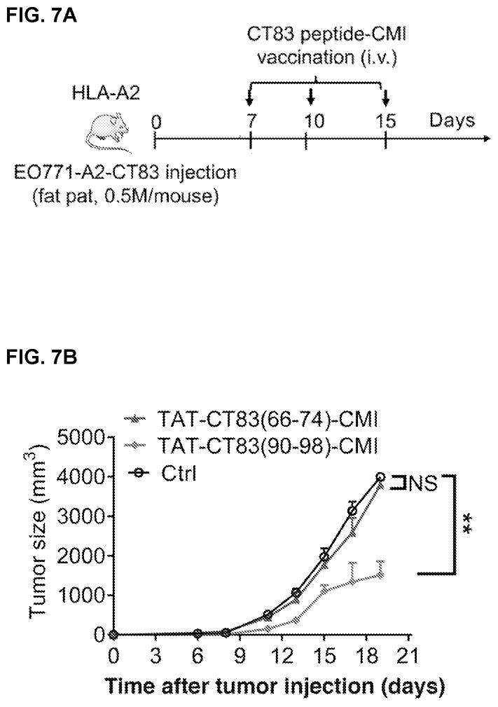

To further test whether the A2-CT83 PEP90-98 can induce antitumor immunity in HLA-A2 transgenic (Tg) mice, E0771-A2-CT83 murine breast cancer cells (0.5×10⁶ cells/mouse) were orthotopically injected into the mammary fat-pad of HLA-A2 mice on day 0. The tumor-bearing mice were treated with self-assembled nanoparticle vaccines containing TAT-CT83 PEP90-98 or TAT-CT83 PEP66-74, along with TLR ligands (CpG, MPLA and poly(I:C), CMI for short) on day 7, 10 and 15 (FIG. 7A). The results surprisingly showed that TAT-CT83 PEP90-98-CMI, but not TAT-CT83 PEP66-74-CMI, significantly inhibited tumor growth (FIG. 7B). CT83 PEP66-74 has been reported to induce T cell response using RNA vaccines³⁵, but no activity was induced for CT83 PEP90-98. Therefore, this was the first demonstration that the CT83 PEP90-98 epitope is capable of inducing T cell response and inhibiting tumor growth in vitro and in vivo.

### Identification and Characterization of A2-CT83 TCRs Using Single Cell Barcoding Technology

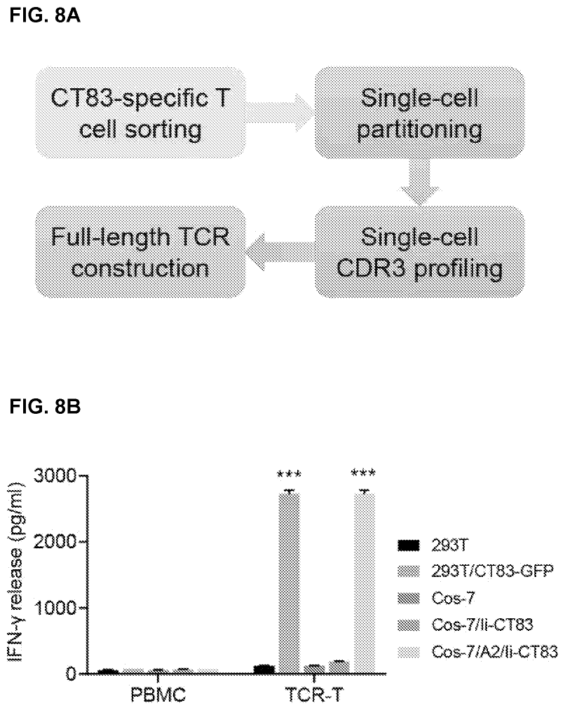

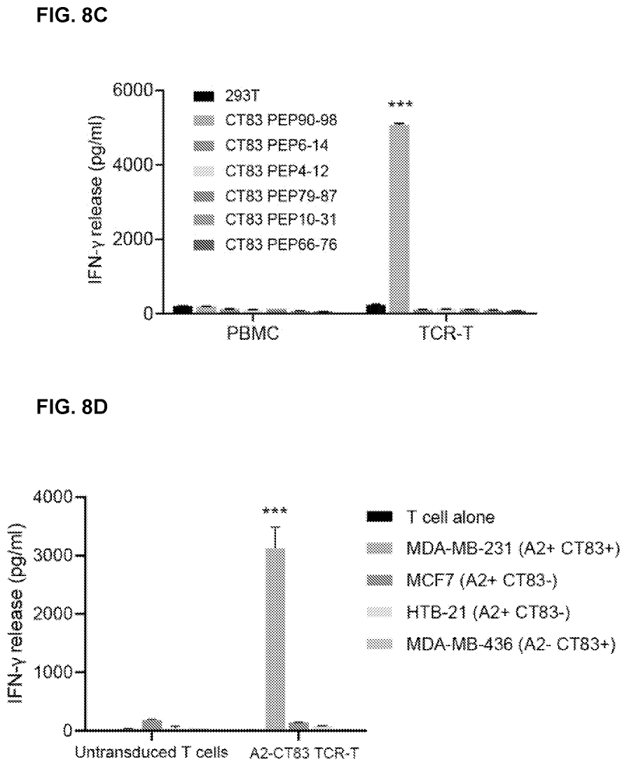

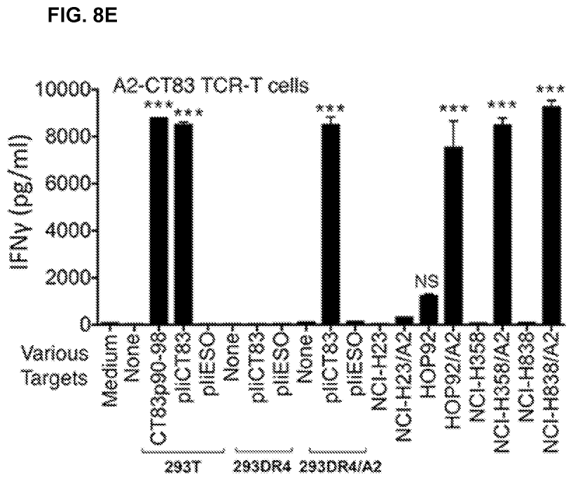

To identify TCRs specific for CT83, A2-CT83-specific T cells were purified by FACS sorting after stimulation of them with 293T/CT83 cells and intracellular staining with anti-IFN-γ (FIG. 8A). The purified T cells (several thousands) were partitioned into nanoliter-scale Gel Beads-in-emulsion (GEMs) on Chromium Next GEM Chip G which was processed in a 10× Genomics Chromium Controller. After multiple steps of cell lysis, reverse transcription (RT), PCR application and Barcoded cDNA for TCR V(D)J library construction according to the manufacture's protocols, and the final enriched TCR V(D)J library was sequenced using an Illumina sequencer (HiSeq 2500). After TCR sequence alignment and analysis, dominant and subdominant paired TCRα and TCRβ were identified, and then full-length TCRα and TCRβ using TCR subtype-specific primers were generated. These full-length TCRs were cloned into retroviral expression vector pMSGV1 or lentiviral expression vector pFU3W (U3 promoter-driven) (FIG. 8A). The results demonstrated that A2-CT83 TCR-T cells were capable of recognizing 293T cells transfected with CT-83-GFP and Cos-7 cells transfected with HLA-A2 and CT83, but not 293T, Cos-7 or Cos-7 transfected with HLA-A2 only (FIG. 8B). The results further showed that A2-CT83 TCR-T cells recognized 293T/CT83 PEP90-98, but not 293T cells or 293T cells pulsed with other CT83 peptides (FIG. 8C). To demonstrate whether these A2-CT83 TCR-T cells are capable of recognizing HLA-A2 and CT83 expressing MDA-MB-231 cells, we co-cultured these T cells with different breast cancer cells. Indeed, A2-CT83 TCR-T cells specifically recognized MDA-MB-231 cells, but not MCF7 (A2⁺ CT83⁻), HTB-21 (A2⁺ CT83⁻) or MDA-MB-436 (A2⁻ CT83⁺) cells (FIG. 8D). Furthermore, A2-CT83 TCR-T cells were tested for their ability to recognize lung cancer cells, and the results showed that these A2-CT83 TCR-T cells could specifically recognize CT83+ and HLA-A2+ lung cancer cells (HOP92/A2, NCI-H358/A and NCI-H838/A2), but not CT83+ HLA-A2− tumor cells (HOP92, NCI-H358 and NCI-838) (FIG. 8E). These results indicate that the A2-CT83 TCR is capable of specifically recognizing the naturally processed CT83 epitope by HLA-A2, which is identified in this invention, in both transfected cell models and natural tumor cell lines.

### Antitumor Activity of A2-CT83 TCR-T Cells In Vivo

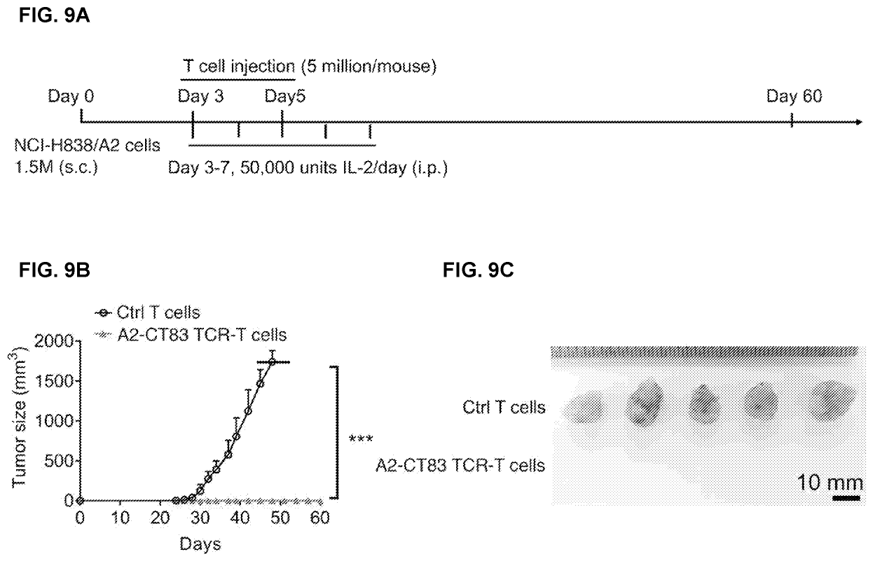

To further demonstrate the antitumor activity of A2-CT83 TCR-T cells in vivo, immunocompromised NSG mice were used as a tumor model. NCI-H838/A tumor cells were injected on day 0, followed by administration of A2-CT83 TCR-T cells (5×10e6 per mouse, i.v.) on day 3 and 5, along with IL-2 (50,000 international units/day, i.p.) from days 3-7 (FIG. 9A). The results showed that A2-CT83 TCR-T cells completely inhibited tumor growth (FIGS. 9B and 9C). By contrast, tumor-bearing mice treated with control T cells developed large tumor mass (FIGS. 9B and 9C). These studies suggest that A2-CT83 TCR-T cells have potent antitumor activity in vivo.

### Example 3 Identification, Construction and Application of A2-HCMV TCRs

Nucleic acids and proteins of human cytomegalovirus (HCMV) could be detected in glioblastoma. Two proteins (pp65 and IE-1) of HCMV particles were targeted for antigen-specific TCR recognition.

### Selection of HLA-A2-Restricted Pp65- and IE-1-Specific T Cells and Clones

The stimulation of pp65- and IE-1-specific T cells was similar as DP4-ESO-1 T cells. CD8+ T cells isolated from PBMCs from healthy donors (with HLA-A2 typing) were pulsed and stimulated with two epitopes (pp65 (495-503) and IE-1 (316-324)) with HLA-A2 restriction respectively. The peptides encoding the two epitopes were synthesized with >95% purity. Matured dendritic cells isolated from autologous PBMCs were resuspended in T cell culture medium and pulsed with the peptides at a concentration of 10 ug/ml overnight respectively. Pulsed cells were than irradiated for 3 min at 60Gy and co-cultured with CD8+T cells at a ratio of 1:5. Primed CD8+ T cells were re-stimulated with 10 ug/ml peptides at day 7 and harvested on day 14.

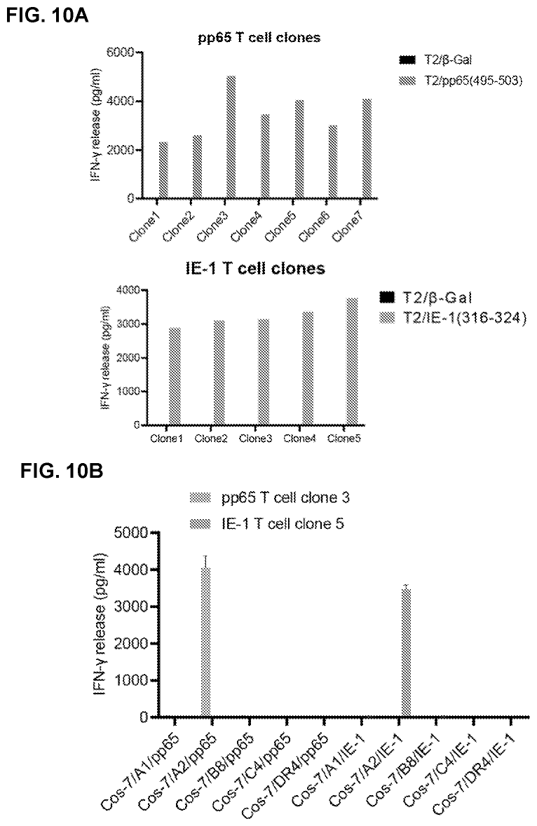

After in vitro stimulation, peptide-reactive CD8+ T cells (15% for pp65 and 8% for IE-1) were sorted, for generating T cell clones by limited dilution (FIG. 10A and FIG. 10B). Seven T cell clones specifically recognized pp65 (495-503) and five T cell clones specifically recognized IE-1 (316-324) when the peptides were loaded on T2 cells, in contrast to β-gal control peptide (FIG. 10A). T cell clone #3, reactive to pp65, and T cell clone #5, reactive to IE-1, were selected for further in vitro activity test respectively. The two T cell clones specifically recognized Cos-7 cells co-transfected with HLA-A2 and pp65, or HLA-A2 and IE-1, respectively, compared to the transfection with other HLA molecules (FIG. 10B). These two T cell clones were used for further TCR cloning.

### Identification, Cloning and In-Vitro Activity of A2-Pp65 TCR and A2-IE-1 TCR

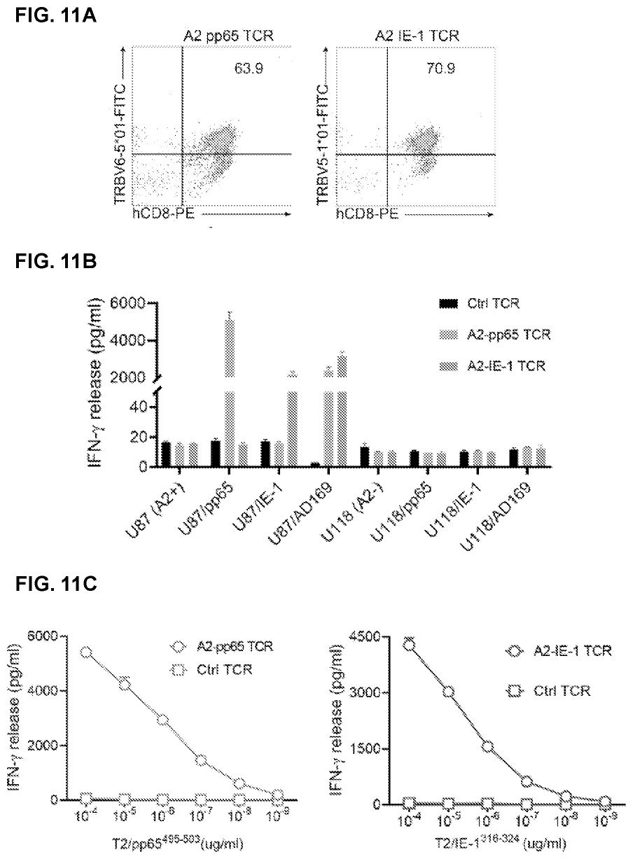

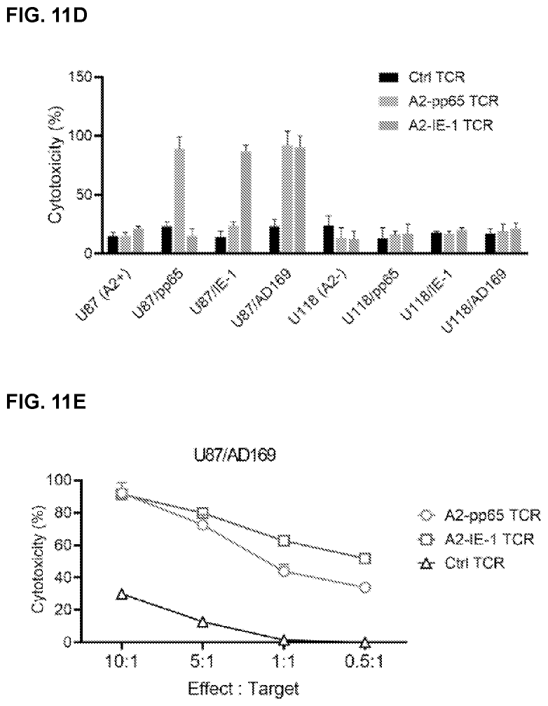

Identification of A2-pp65 TCRs and A2-1E-1 TCRs from T cell clones was performed using methods similar to those described above. After CDR3 sequencing, the TCR repertoires of both TCRs (TRAV24, TRBV6-5 of pp65 T cell clone #3; TRAV25 and TRBVS-1 of 1E-1 T cell clone #5) were identified. The full-length TCRs were amplified with specific primers and then constructed into the pMSGV vector, respectively. The retroviral TCR transductions in human T cells were conducted as described above. The transduction efficiency of both TCRs were checked by FACS after staining with TCR-specific antibodies respectively. Both TCRs showed transduction efficiency >60% (FIG. 11A), which indicated good candidates for in vitro assays. After incubation, A2-pp65 TCR-transduced or A2-IE-1 TCR-transduced T cells were able to recognized U87 glioblastoma line (HLA-A2+) transfected with pp65 or IE-1 respectively, but had no recognition against U118 glioblastoma line (HLA-A2−) transfected with pp65 or IE-1 (FIG. 11B). Particularly, both TCR-transduced T cells specifically recognized U87 cell line infected by HCMV strain AD169, compared to U118 cell line after infection (FIG. 11B). Both TCR-transduced T cells had dose-dependent recognition patterns with T2 cells pulsed with pp65 (495-503) or IE-1 (316-324) peptides, respectively (FIG. 11C). To determine the cytolytic ability, cytotoxicity of A2-pp65 TCR-transduced or A2-IE-1 TCR-transduced T cells were assayed as well. Both TCR-transduced T cells showed almost 100% cell lysis on U87 cells either transfected with pp65 or IE-1 or infected by HCMV strain AD169 respectively (FIG. 11D). The cytotoxicity of both TCR-transduced T cells on AD169-infected U87 cells also proceeded in a dose-dependent manner (FIG. 11E).

### Antitumor Activity of A2-Pp65 TCR and A2-IE-1 TCR Transduced T Cells In Vivo

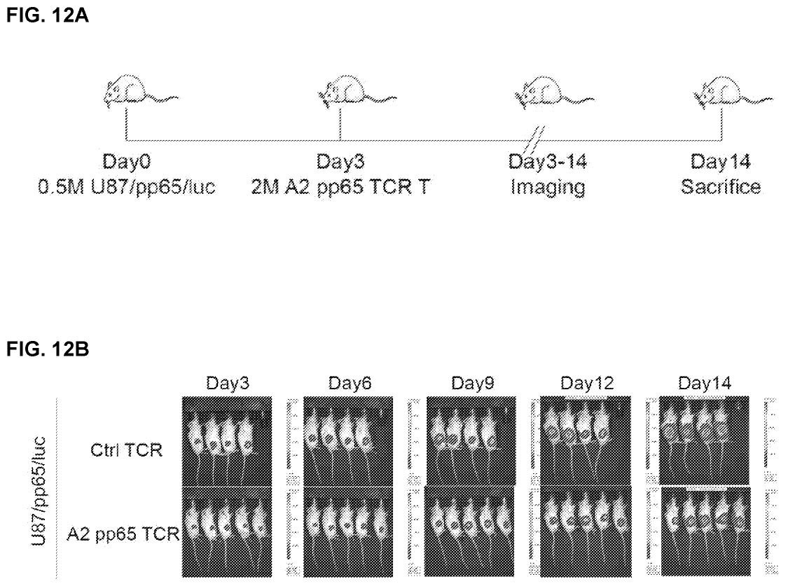

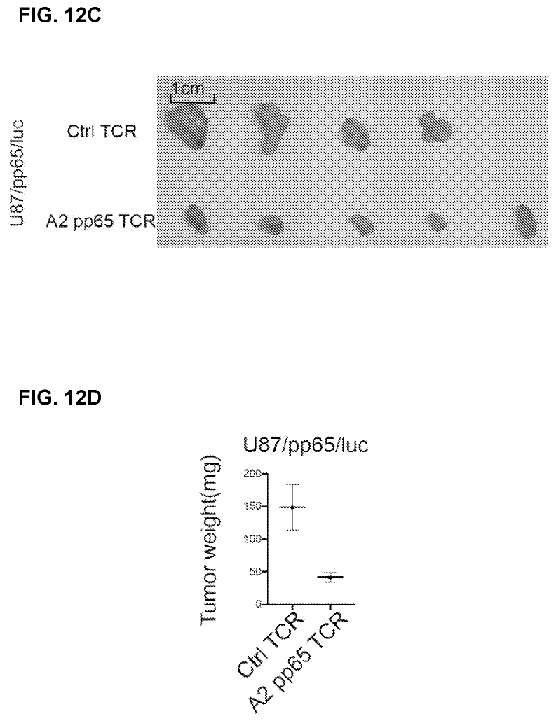

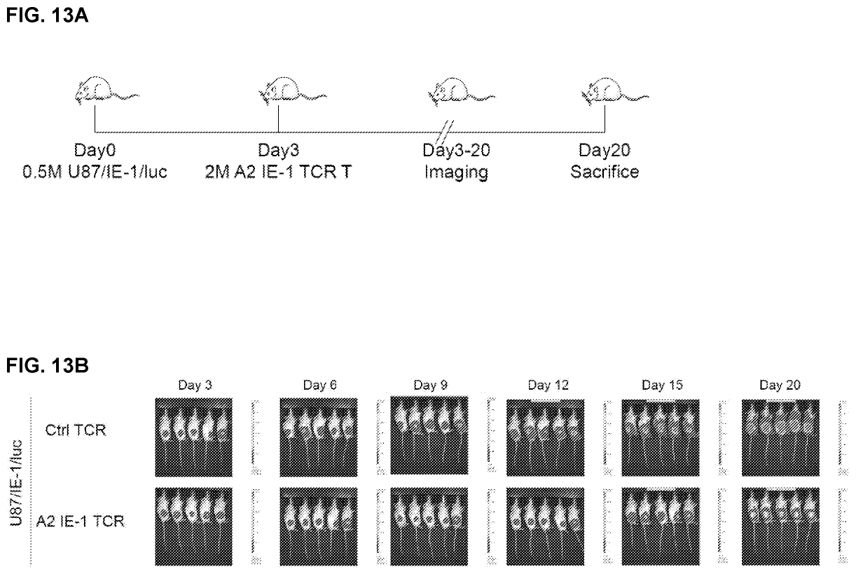

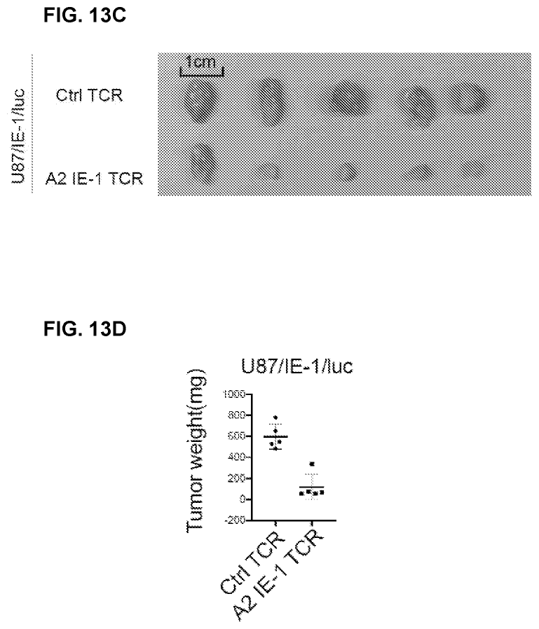

To assess the antitumor activity of HCMV antigen-expressing tumors by A2-pp65 TCR-transduced and A2-IE-1 TCR-transduced T cells in vivo, we used a xenograft model in which transplanted tumors were established in immunodeficient mice with U87 cells expressing pp65 or IE-I, and luciferase. Tumors were established in SCID/Beige mice for 3 days and then treated by adoptive transfer of A2-pp65 TCR, A2-IE-1 TCR or control TCR transduced human T cells by i.v. injection of 2×10⁶ T cells per mouse (FIG. 12A and FIG. 13A). The migration of injected T cells was observed every 3 days until sacrifice of the mice (FIG. 12B and FIG. 13B). Tumor growth was efficiently suppressed (n=5) in both TCR treatment groups compared to the control group receiving control TCR-transduced T cells (n=3) from the same healthy donor. A tumor size decrease was observed in all animals treated with A2-pp65 TCR-transduced or A2-IE-I TCR-transduced T cells (FIG. 12C, 12D and FIG. 13C, 13D). These results indicate the antitumor activity of both TCRs and their further application in the treatment of glioblastoma.

### Example 4 Reduced TCR Mispairing with Endogenous TCRs to Improve TCR Expression, Specificity and Function

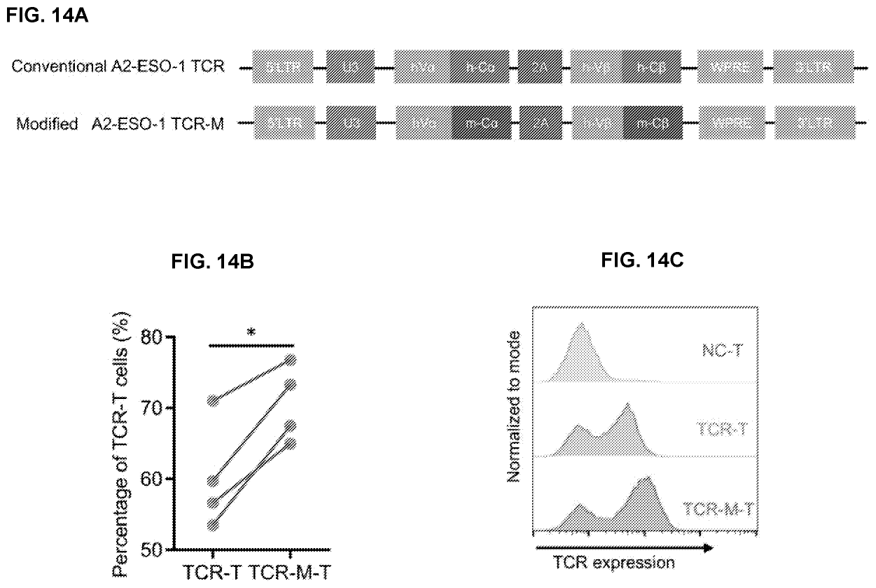

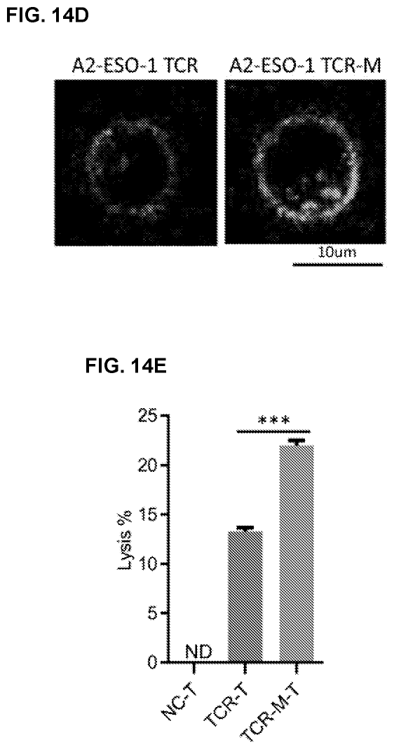

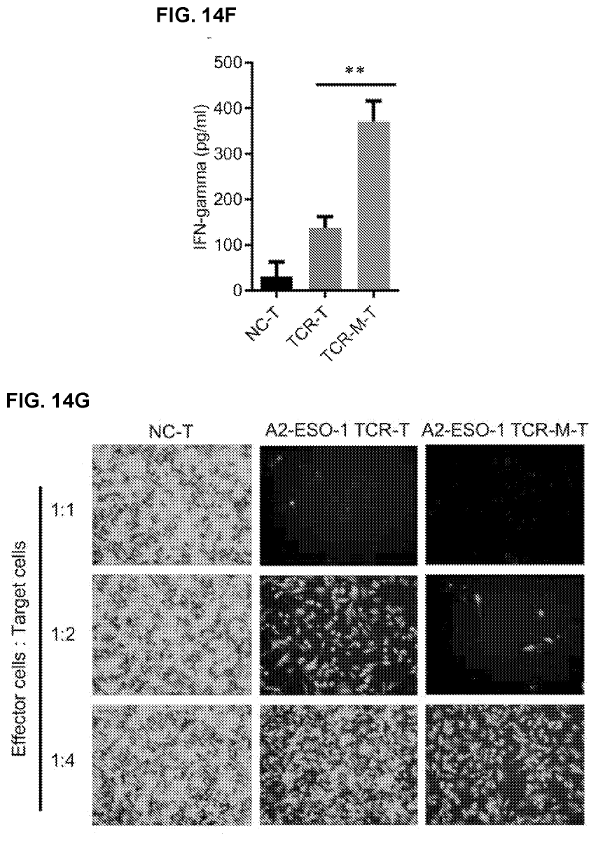

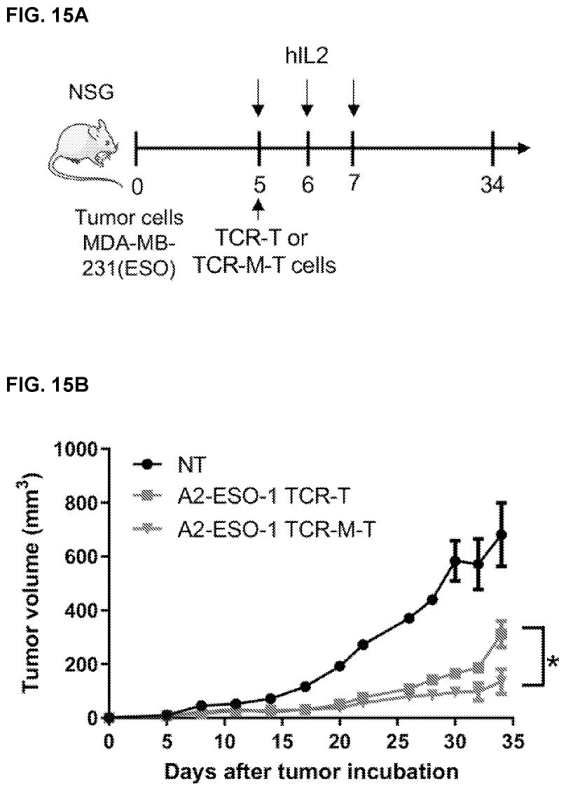

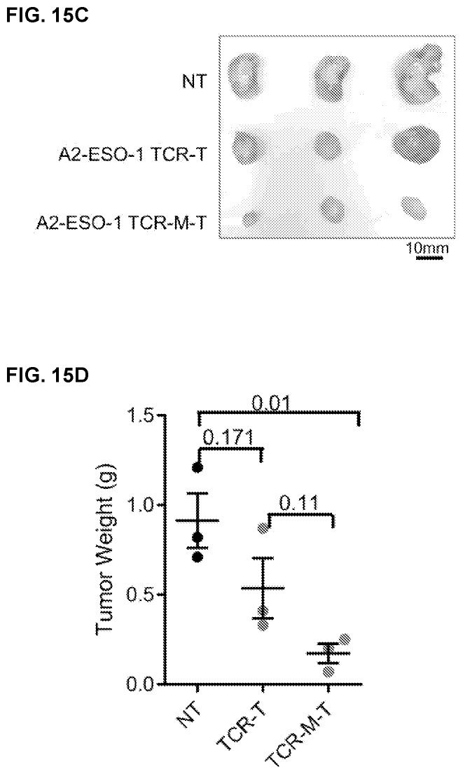

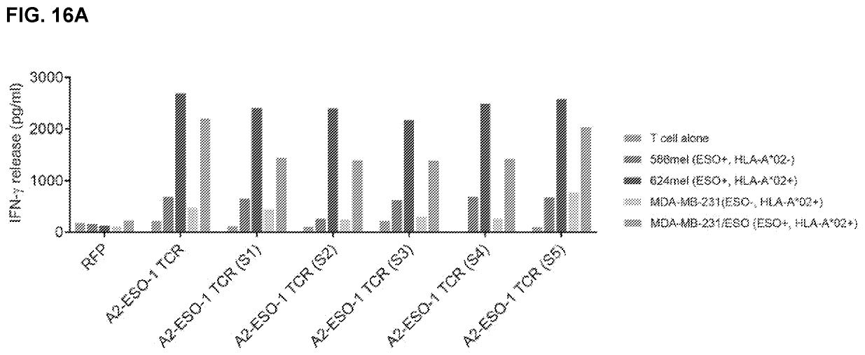

Because each T cell contains its own endogenous TCR, it is likely that CT83-specific TCRα and TCRβ may mispair with endogenous TCRα and TCRβ, which either generates a TCR(α/β) which is non-functional, or a new TCR(α/β) with unexpected antigen specificity. Spear, T. T., Foley, K. C., Garrett-Mayer, E. & Nishimura, M. I. TCR modifications that enhance chain pairing in gene-modified T cells can augment cross-reactivity and alleviate CD8 dependence. J. Leukoc. Biol. 103, 973-983 (2018; Bethune, M. T. et al. Domain-swapped T cell receptors improve the safety of TCR gene therapy. Elife 5 (2016). Two strategies are useful to avoid this problem: one approach is to knockout endogenous TCR(α/β) using CPRISPR/Cas9 technology (Legut, M., Dolton, G., Mian, A. A., Ottmann, O. G. & Sewell, A. K. CRISPR-mediated TCR replacement generates superior anticancer transgenic T cells. Blood 131, 311-322 (2018)); an alternative approach is to generate chimeric TCRs by fusing the NY-ESO-1 or CT83 TCR variable region to a non-human, e.g., murine TCR constant region, thus reducing the mispairing between endogenous TCR (HC) and NY-ESO-1 TCR (MC) or CT83 TCR(MC). Using the latter approach, it was demonstrated that replacement of human NY-ESO-1 TCR with murine constant region improved TCR expression and functional recognition of tumor cells (FIG. 14A-14G). Importantly, A2-ESO-1 TCR-M engineered T cells showed stronger antitumor activity in vivo than A2-ESO-1 TCR engineered T cells (FIG. 15A-15D).

### A2-ESO-1 TCR and A2-CT83 TCRs by Use of Murine TCR Constant Regions

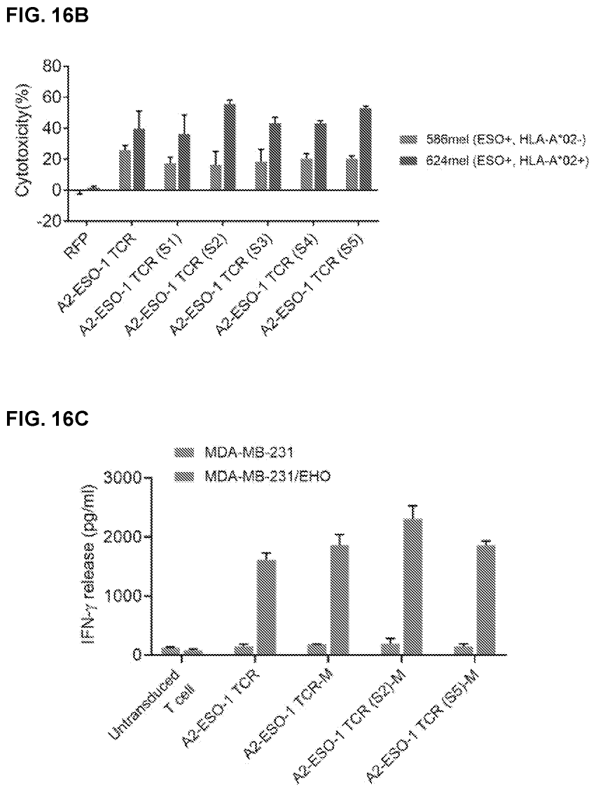

Five constructs of A2-ESO-1 TCR were engineered with different amino acid substitutions in variable region of alpha or beta chains. The pMSGV-A2-ESO-1 TCR construct was used as template for PCR amplification of TCR fragments with primers containing site-directed mutations. After overlapping of multiple PCR amplicons, five variants of A2-ESO-1 were cloned back to pMSGV vector (Sub 1: encoding S53W mutation in alpha chain; Sub 2: encoding G50A, A51E mutations in beta chain; Sub 3: encoding G50A mutation in beta chain; Sub 4: encoding A97L mutation in beta chain; Sub 5: encoding G50A, A51E, A97L mutations in beta chain). The results showed that these A2-ESO-1 TCR-transduced T cells were capable of recognizing NY-ESO-1-expressing 624mel (HLA-A2+) and MDA-MB-231(HLA-A2+), but not 586mel (HLA-A2−) tumor cells (FIG. 16A). Consistently, cytotoxity was demonstrated for the A2-ESO-1 TCR (containing amino acid substitutions)-transduced T cells against NY-ESO-1-expressing 624mel (HLA-A2+) and MDA-MB-231(HLA-A2+) (FIG. 16B).

To reduce potential mispairing between A2-ESO-1 TCRs with endogeneous human TCRs (without defined antigen specificity), human A2-ESO-1 TCR constant regions were replaced with murine TCR constant regions and chimeric A2-ESO-1 TCR (human TCR alpha or beta variable regions fused with murine TCR alpha or beta constant regions, respectively) were generated. It was found that T cells transduced with TCRs with murine constant regions (A2-ESO-1 TCR-M, A2-ESO-1 TCR (S2)-M and A2-ESO-1 TCR (S5)-M) were capable of recognizing MDA-MB-231/ESO cells (FIG. 16C), suggesting that murine constant region-substitution enhances the expression and function of TCRs in human T cells.

### Antitumor Activity of A2-CT83 TCR-M T Cells (Murine Constant Regions)

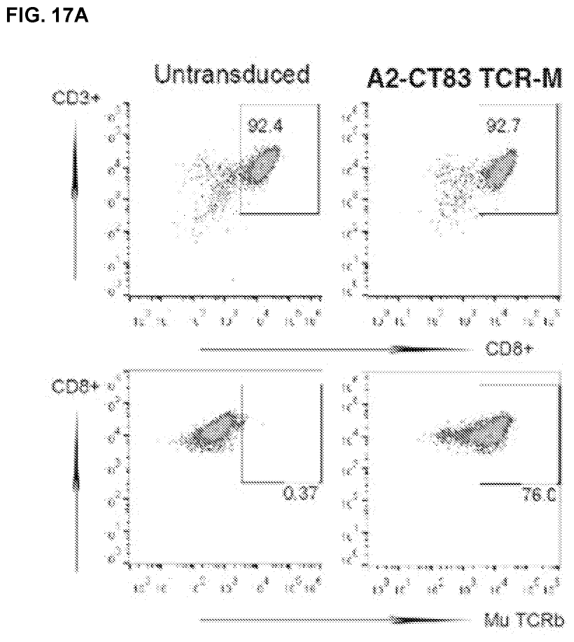

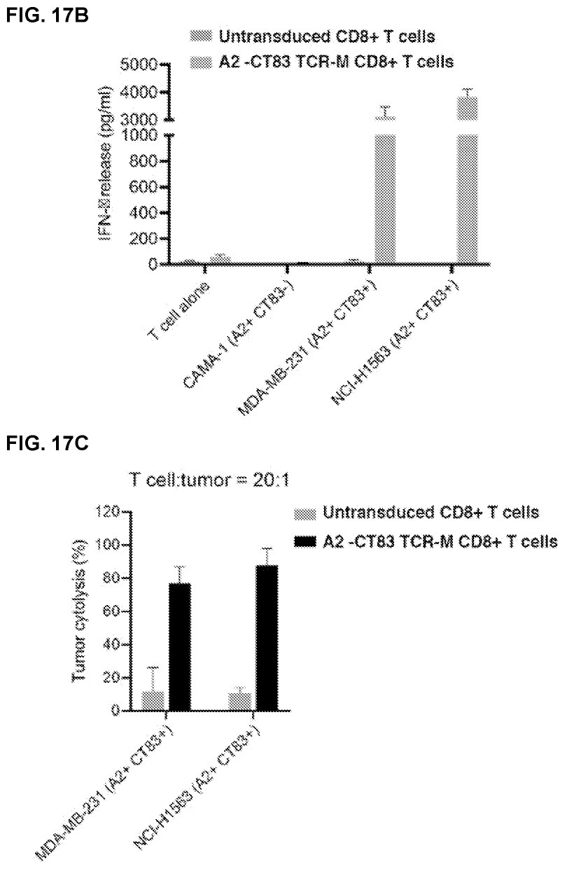

The FACS analysis showed the transduction efficiency of A2-CT83 TCR-M (murine constant regions) in human T cells was above 70% (FIG. 17A). The A2-CT83 TCR-M-transduced T cells strongly recognized HLA-A2+ and CT83+ tumor cells (MDA-MB-231 and NCI-H1563), but did not recognize CT83− tumor cells (CAMA-1) (FIG. 17B). Consistently, the results showed 60-80% of tumor cell lysis against MDA-MB-231 and NCI-H1563 (FIG. 17C). These results indicate that A2-CT83 TCR-M T cells are potent and specific against tumor cells, with reduced TCR mispairing.

### Example 4: Modulation of CAR-T Cell Signaling for T Cell Persistence by Replacement of CD3ζ Signaling with Signaling Domain Derived from a ZAP70 Kinase Domain

### TCR Complexes Comprising Anti-CD19 scFv-CD28-ZAP300 (1928ZAP300) and Anti-CD19-CD28-ZAP327 (1928ZAP327)

CAR constructs contain a single chain variable fragment (ScFv) for antigen recognition, a transmembrane domain, and an intracellular T-cell activation moiety (consisting of CD28 or 4-1BB costimulatory signaling domain fused with CD3ζ signaling domain. Sadelain, M., Brentjens, R. & Riviere, I. The basic principles of chimeric antigen receptor design. Cancer Discov 3, 388-398, doi:10.1158/2159-8290.CD-12-0548 (2013). The CD3ζ chain is one important component of the TCR-CD3 complex, which plays a critical role in signaling transducing. The function of CD3ζ is to recruit Zap70 after phosphorylated in the ITAMs when it is activated after TCR engagement. Current CAR constructs contain a CD3ζ chain for T cell signaling transduction and recruit ZAP70. Experiments were conducted to demonstrate that a CD3z signaling domain could be replaced with other signaling domains to enhance CAR-T cell activation and persistence, for example, to demonstrate that CAR constructs containing a signaling moiety derived from Zap70 would enhance CAR-T cell signaling and CAR-T cell activation and persistence.

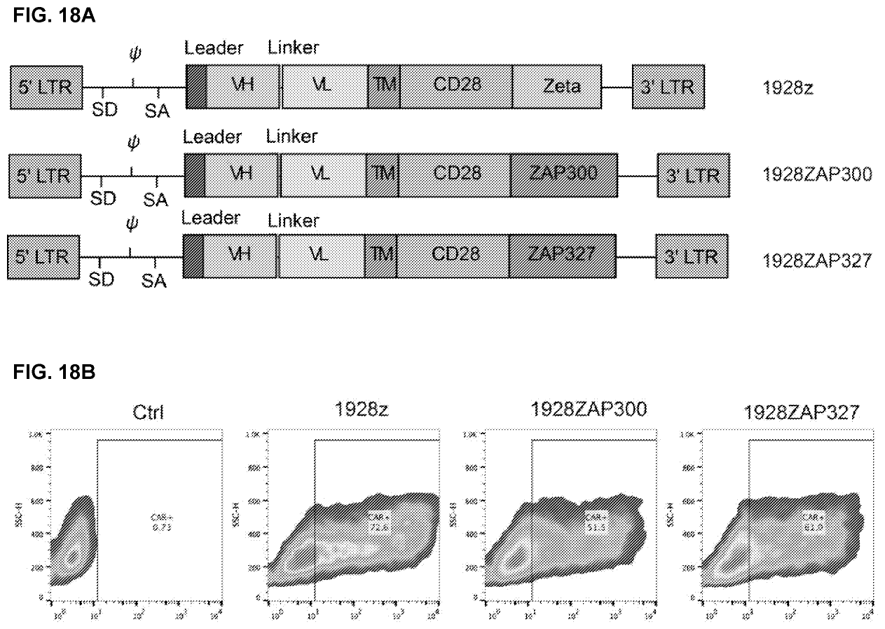

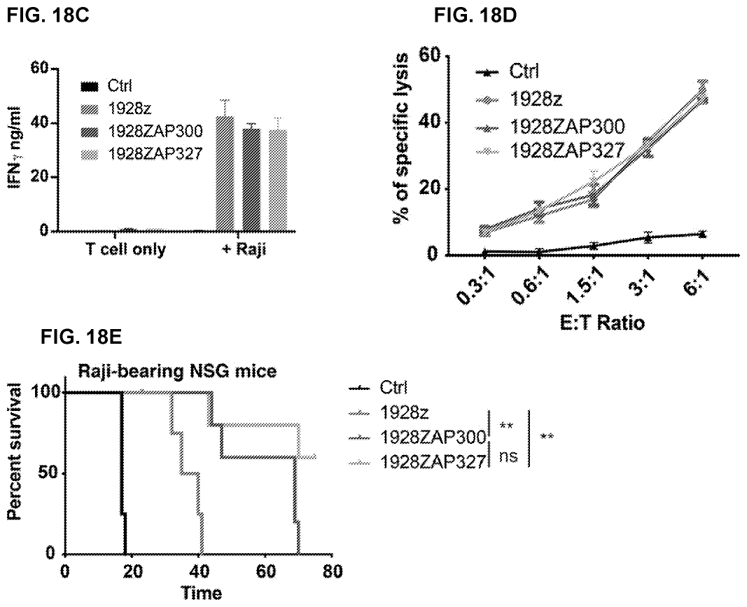

To this end, signaling domains derived from ZAP70 and LAT were screened and anti-CD19 scFv-CD28-ZAP300 (from amino acid residue position 300 to the C-terminal) and anti-CD19-CD28-ZAP327 (position 327 to the C-terminal of the ZAP protein) were identified (FIG. 11A). These Zap70 kinase domains (starting from 300 a.a. and 327 a.a. of Zap70) were fused to the C terminus of CD3 chain with CD28 as costimulatory domain to generate anti-CD19 scFv-CD28-ZAP300 (1928ZAP300) and anti-CD19-CD28-ZAP327 (1928ZAP327) (FIG. 18A). Flow cytometry demonstrated comparable transduction efficiency of 1928ZAP300 and 1928ZAP327 to conventional anti-CD19-CD28-CD3ζ (1928Z for short) for CAR expression (FIG. 18B). Furthermore, these CAR-T cells directly killed Raji tumor target cells in a non-radioactive LDH cytotoxicity assay with a series dilution of E:T ratios (FIG. 18C). Thus, FIGS. 18C and 18D show the antigen-specific recognition and tumor cell lysis after CAR-T cells were cocultured with Raji tumor cells, demonstrating that ZAP300 and ZAP327 CAR-T cells are functional and specific for the target antigen in in vitro experimental studies.

The results further showed that 1928ZAP300 and 1928ZAP327 CAR-T cells outperformed 1928z CAR-T cells in in vivo experiments, and surprisingly and dramatically prolonged overall survival in a Raji lymphoma mouse model (FIG. 18D). Raji-bearing NSG mice were treated with T cells transduced with none, 1928Z, 1928ZAP300 or 1928ZAP327 CAR-T cells, and it was found that tumor-bearing NSG mice treated with control T cells died within 20 days after tumor injection. Treatment of tumor-bearing mice with 1928Z CAR-T cells died around day 40. By contrast, treatment of tumor-bearing mice with 1928ZAP300 or 1928ZAP327 CAR-T cells significantly prolong mouse survival to 70 days or more after tumor injection (FIG. 18E). In particular, more than 60% of tumor-bearing mice treated with 1928ZAP327 CAR-T cells survived in more than 80 days. These studies indicate that replacing CD3ζ chain with Zap70 kinase domains (ZAP300 and ZAP327) markedly enhances antitumor activity in vivo.

### 19bbZAP327 CAR-T Cells Produce Low Amounts of Cytokines and More Potent Antitumor Immunity

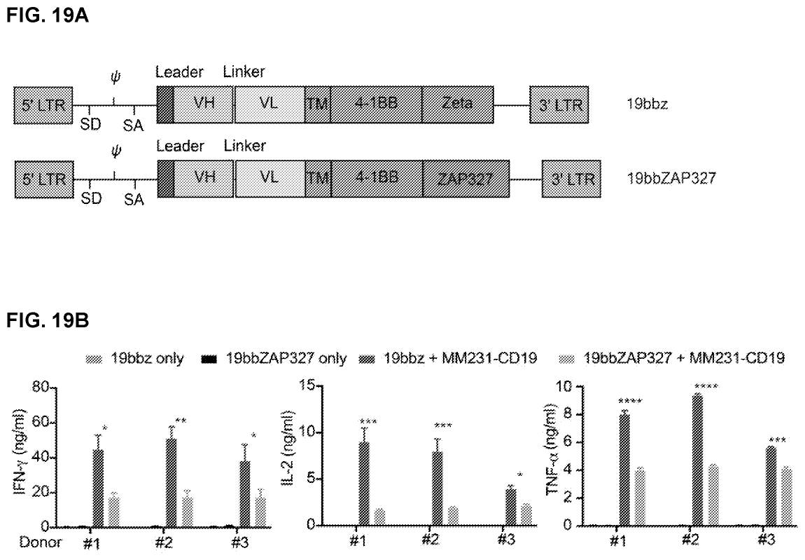

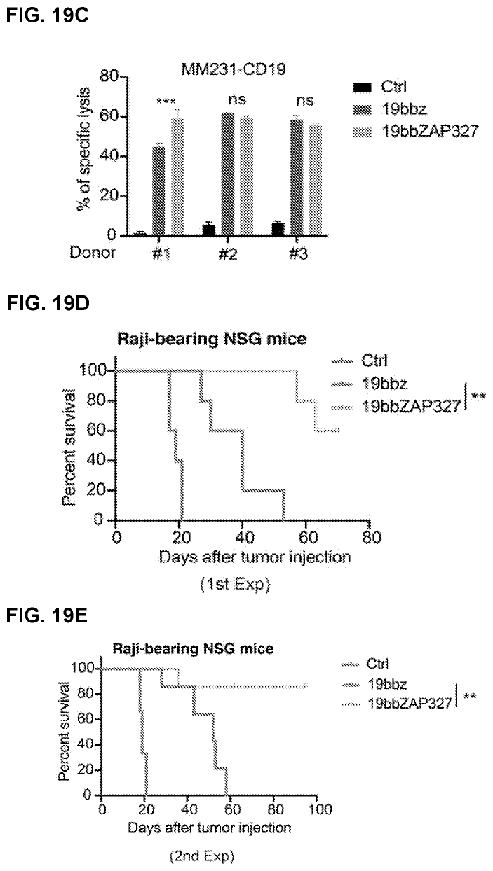

To test whether Zap70 kinase domain could also work in 4-1BB containing CAR construct, a CAR construct was generated with a Zap70 kinase domain (e.g., ZAP327) fusion to 4-1BB domain (19bbZAP327) (FIG. 19A). The results showed that 19bbZAP327 CAR-T cells produced significantly lower amounts of IFN-γ, IL-2 and TNF-a, compared with 19bbz CAR-T cells after stimulation with tumor cells (FIG. 19B). 19bbZAP327 and 19bbz CAR-T cells showed comparable specific tumor lysis in vitro (FIG. 19C). Importantly, tumor-bearing mice treated with 19bbZAP327 demonstrated superior antitumor activity compared to 19bbz CAR-T cells (FIGS. 19D and 19E). Mice treated with 19bbZAP327 CAR-T cells markedly prolonged mouse survival compared with mice treated with 19bbZ CAR-T cells (FIGS. 19D and 19E). Taken together, our results show that 19bbZAP327 CAR-T cells produce low amounts of cytokines and more potent antitumor immunity, compared with conventional 19bbz CAR-T cells.

Furthermore, ZAP327 could be fused to TCR constructs to enhance T cell signaling. These studies demonstrate that ZAP327 signaling domain can enhance CAR-T and TCR-T cell signaling, function and persistence. In other embodiments, ZAP300 or other signaling domain derived from ZAP70 may be used in CAR or TCR constructs to enhance anti-tumor activity whie reducing the amounts of cytokines produced.

### ZAP327 Signaling Domain Promotes T Cell Memory Function and Persistence In Vivo

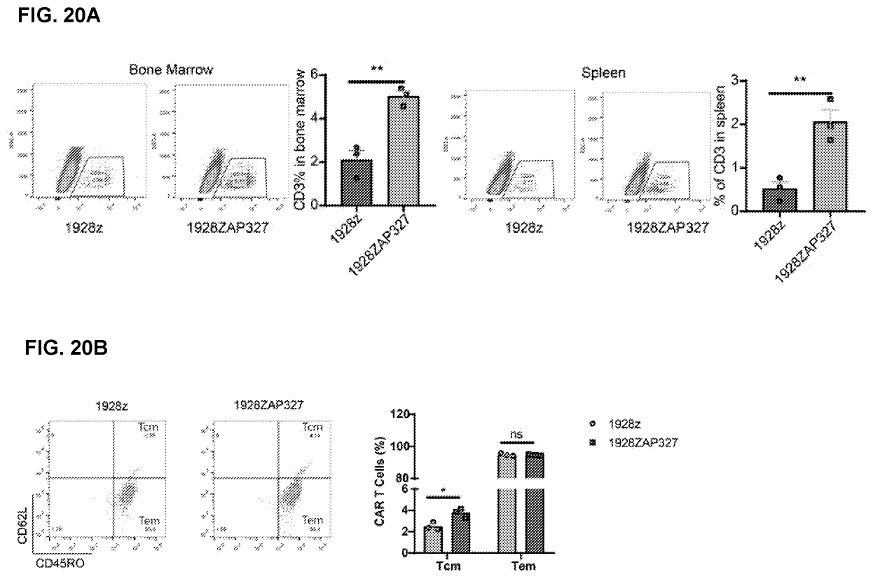

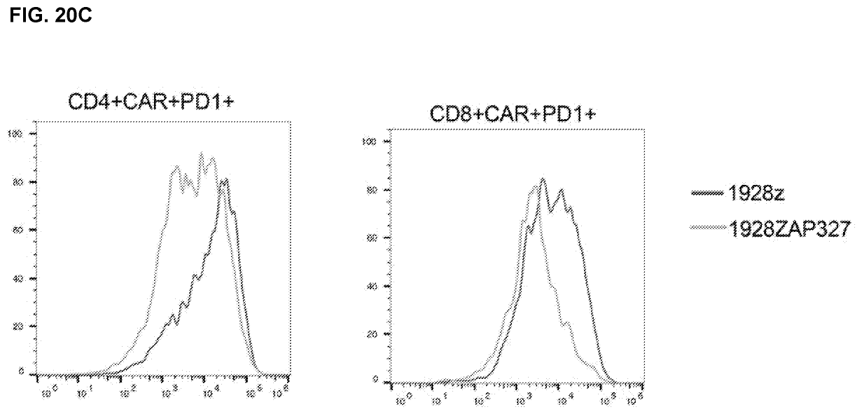

To understand why 1928ZAP327 CAR-T cells produce more potent antitumor immunity, we tested T cell survival at day 30 after T cell transfer. We showed that higher percentages of 1928ZAP327 CAR-T cells in bone marrows and spleens, compared with mice treated with 1928z 30 days after T cell transfer (FIG. 20A). Furthermore, the percentages of central memory 1928ZAP327 CAR-T cells were higher than 1928z CAR-T cells (FIG. 20B). 1928ZAP327 CAR-T cells expressed lower amounts of PD-1 molecules (an exhaustion marker) than 1928z CAR-T cells (FIG. 20C), suggesting that ZAP327 signaling domain promotes T cell memory function and persistence in vivo. In other embodiments, ZAP300 or other signaling domain derived from ZAP70 may be used in CAR or TCR constructs to promote T cell memory function and persistence in vivo.

### Example 6 Modulation of TCR-T Cell Function In Vivo by Knocking Down the Expression of Metabolic and Epigenetic Genes PD1, VHL, PPP2R2D or JMJD3

### Knockdown of PD1, VHL, PPP2R2D Enhance TCR-T Cell Function

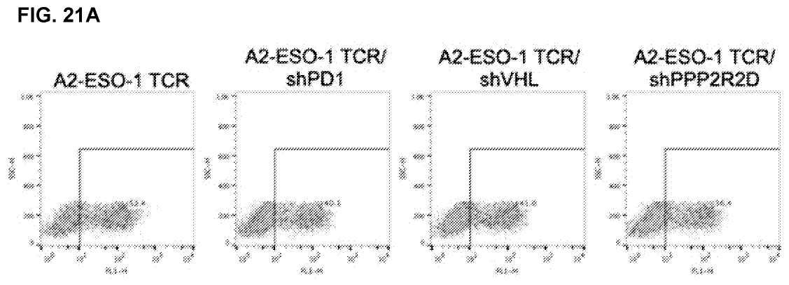

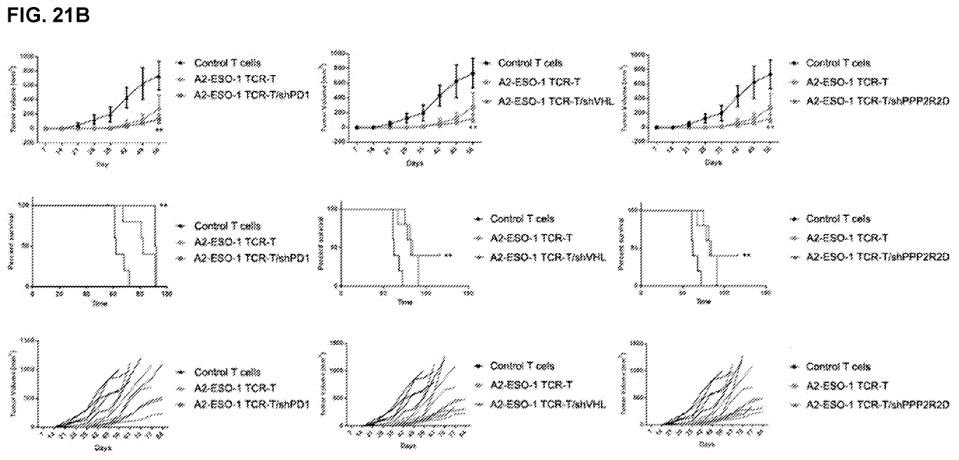

The shRNAs of PD1, VHL and PPP2R2D were constructed and cloned into the downstream of A2-ESO-1 TCR expressing vector. The retroviral particles were produced and used to transduce naive human T cells. The transduction efficiency was determined by A2-ESO-1 TCR staining and FACS analysis. We showed that transduction efficiency of T cells (FIG. 21A) and used for animal experiments. Breast cancer-bearing mice were generated by s.c. injection of engineered MDA-MB-231/NY-ESO-1/luciferase cells into NSG mice. Three days later, naive T cells, A2-ESO-1 TCR-T cells with or without knockdown of PD1, VHL and PPP2R2D were intratumorally injected. Tumor burden was assessed by luciferase in vivo imaging at indicated time points and the mice survival was monitored (FIG. 21B). All A2-ESO-1 TCR-T cells with PD1, VHL or PPP2R2D knockdown respectively showed better or similar tumor suppression than A2-ESO-1 TCR-T cells alone. Combining with PD1, VHL or PPP2R2D knockdown, TCR-T cells showed longer mice survival time. In VHL and PPP2R2D groups, some mice even survived till the experiment ended (FIG. 21B).

### Knockdown or Knockout of JMJD3 Enhances T Cell Function and Persistence In Vivo

In addition, we show that CAR constructs containing with JMJD3 shRNA or LSD1 shRNA could prolong T cell persistence (memory T cell function), thus enhancing antitumor immunity and mouse survival.

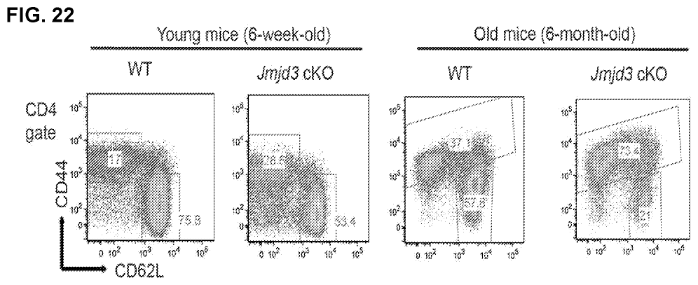

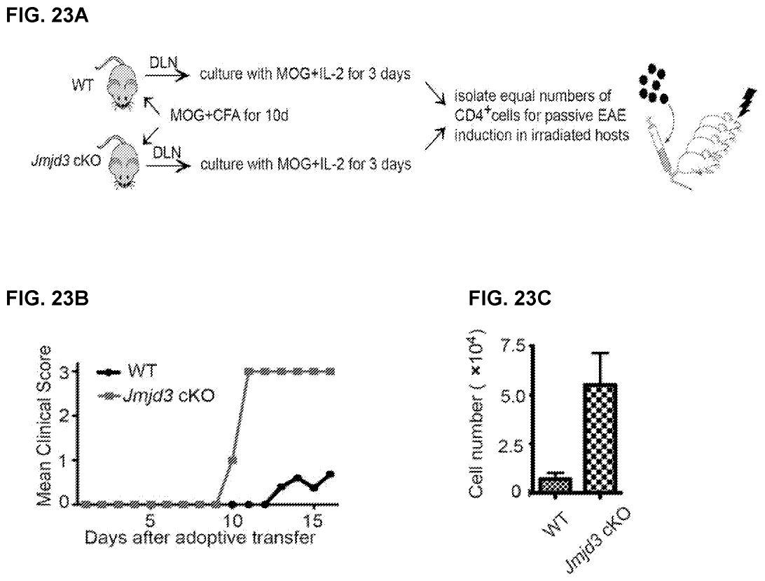

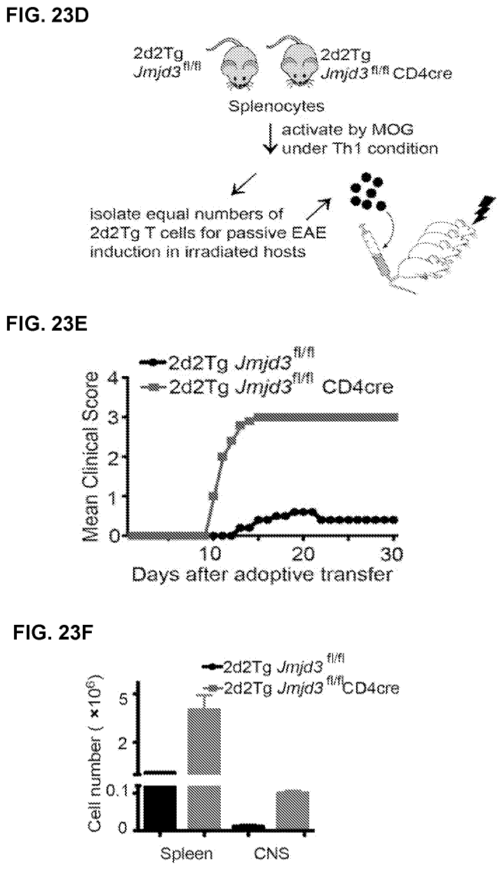

As described herein, it was demonstrated that Jmjd3 conditional knock out (KO) in CD4+ T cells enhanced CD44+CD62L− memory T cell population, compared with wild-type (WT) mice (FIG. 22). When WT and Jmjd3 cKO T cells were used, the results further showed that Jmjd3 cKO 2d2 transgenic CD4+ T cells stimulated in vivo with MOG peptide plus complete Freund's adjuvant (FIG. 23A) markedly enhanced clinical scores in an EAE mouse model (FIG. 23B), which was closely associated higher number of Jmjd3 cKO T cells, compared with WT 2dT cells after T cell transfer (FIG. 23C). Similar results were obtained using an in vitro T cell stimulation (FIGS. 23D, 23E and 23F). These results indicate that Jmjd3 KO markedly enhances T cell survival and persistence.

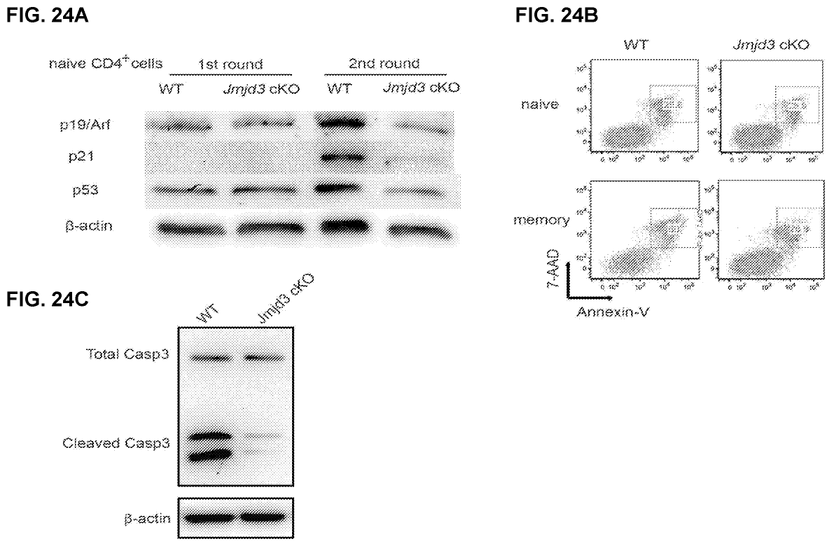

To determine the molecular mechanisms responsible for the enhanced T cell survival and persistence, it was further demonstrated that Jmjd3 cKO T cells markedly reduced the levels of p19, p21 and p53 (key proteins regulating T cell apoptosis) after 2nd stimulation with anti-CD3 and CD28 antibodies (FIG. 24A). Indeed, the results showed that Jmjd3 cKO T cells had much lower levels of T cell apoptosis, compared with WT T cells after 2nd stimulation with anti-CD3 and CD28 antibodies (FIG. 24B). To provide direct evidence for reduced T cell apoptosis in Jmjd3 cKO T cells, it was also determined that Jmjd3 cKO T cells had very low level of cleaved Caspase 3, compared with WT T cells after anti-CD3 and CD28 stimulation (FIG. 24C). These results indicate that Jmjd3 KO markedly enhances T cell survival and persistence by reducing Caspase 3 activation.

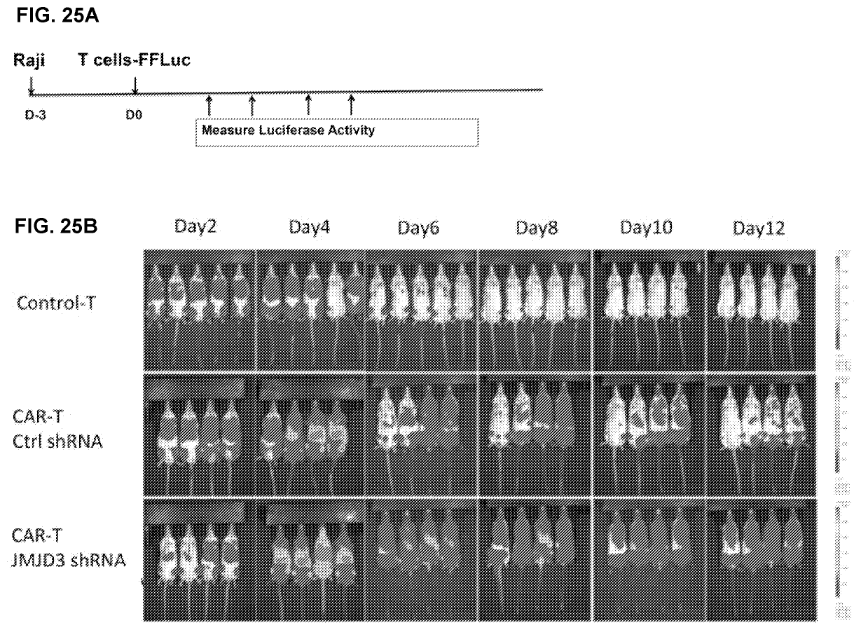

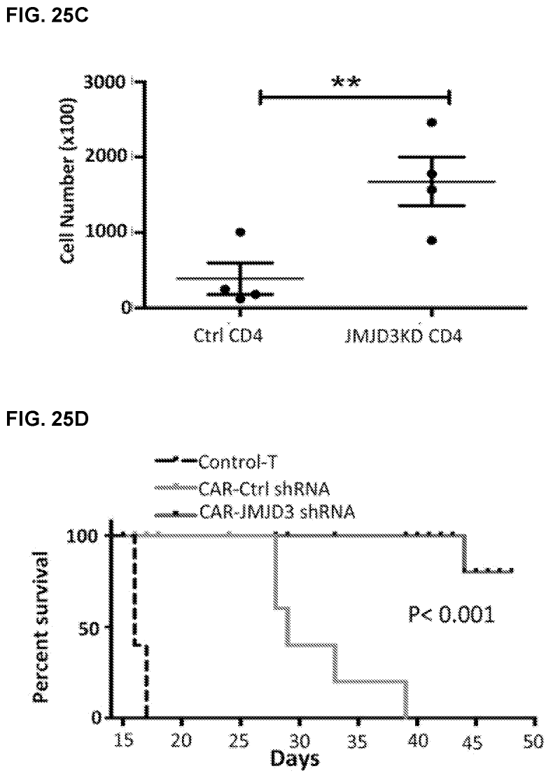

Next, it was determined whether knockdown of JMJD3 enhances CAR-T cell survival and persistence, thus augmenting antitumor immunity. FIG. 25A shows experiment design using Raji tumor cells for monitoring luciferase-labeled T cell survival. 1928z-shJMJD3 CAR-T cells showed strong proliferation at day 4 after T cell transfer into Raji tumor-bearing NSG mice, but maintained high levels of T cells (FIGS. 25B and 25C). By contrast, 1928z-control-sh CAR-T cells markedly reduced T cell number after 6 days of T cell transfer (FIGS. 25B and 25C). Consistent with these observations, the results showed that 1928z-shJMJD3 CAR-T cells markedly inhibited tumor growth and prolong mouse survival, compared with mice treated with 1928z-control sh CAR-T cells (FIG. 25D).

These studies indicate that knockdown or knockout of negative regulators or epigenetic factors can modulate CAR-T and TCR-T cell function, persistence and antitumor immunity in vivo.

### Example 7 Redirecting T Cell Trafficking to Tumor Sites by Forced Expression of Chemokine Receptors

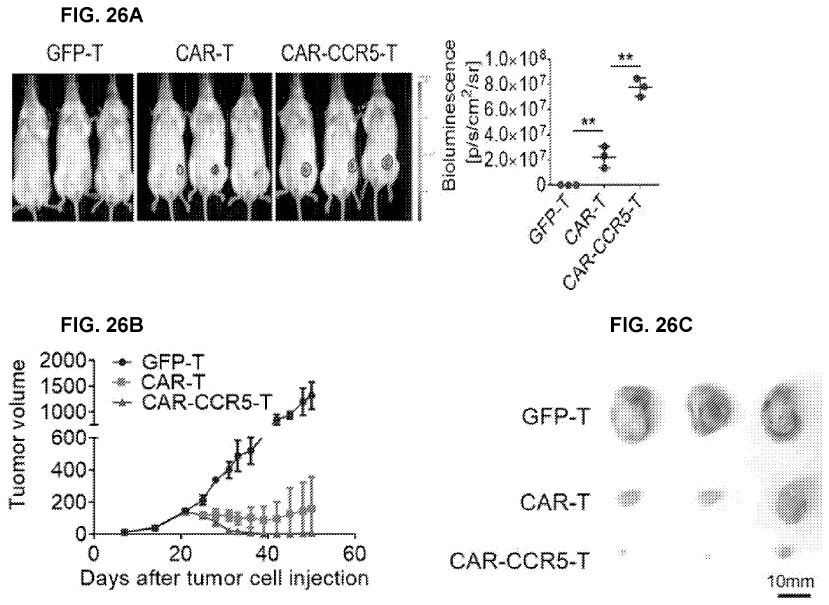

To study the function of chemokine receptors in anti-tumor immunity, CAR constructs with chemokine receptor expression were engineered. The results showed that CCR5 could significantly enhanced 1928z CAR-T cell trafficking into the tumor sites, compared with control tumor-specific T cells (FIG. 26A). Using MDA-MB-231/CD19 tumor cells, it was demonstrated that 1928z-CCR5 CAR-T cells surprisingly markedly inhibited growth of solid tumor cells (FIGS. 26B and 26C). These results suggest that forced chemokine receptor expression enhances T cell trafficking into tumor cells.

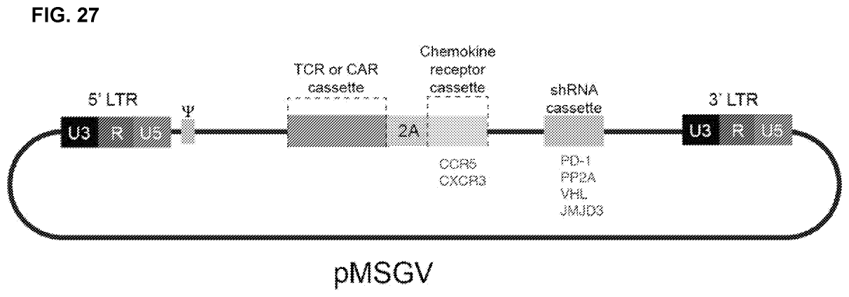

To further enhance T cell survival once they migrate into tumor sites, we could combine T cell trafficking with T cell persistence using a strategy illustrated in FIG. 27. Chemokine receptor and shRNA KD could be inserted into TCR or CAR constructs.

### F. Sequences

SEQ ID NO: 1
HLA-DP4-restricted NY-ESO-1 epitope (157-170)
SLLMWITQCFLPVF
SEQ ID NO: 2
HLA-A2-restrcited CT83 epitope (90-98)
KLVELEHTL
SEQ ID NO: 3
HLA-DP4-redtricted NY-ESO-1 TCR Alpha chain variable domain (TRAV34-TRAJ26)
METVLQVLLGILGFQAAWVSSQELEQSPQSLIVQEGKNLTINCTSSKTLYGLYWYKQ
KYGEGLIFLMMLQKGGEEKSHEKITAKLDEKKQQSSLHITASQPSHAGIYLCGADIVD
YGQNFVFGPGTRLSVLPY
SEQ ID NO: 4
HLA-DP4-redtricted NY-ESO-1 TCR Beta chain variable domain (TRBV30-TRBJ2-7)
MLCSLLALLLGTFFGVRSQTIHQWPATLVQPVGSPLSLECTVEGTSNPNLYWYRQAA
GRGLQLLFYSVGIGQISSEVPQNLSASRPQDRQFILSSKKLLLSDSGFYLCAWRRRGY
EQYFGPGTRLTVTE
SEQ ID NO: 5
HLA-A2-restrcited CT83 TCR Alpha chain variable domain (TRAV5-TRAJ28)
MKTFAGFSFLFLWLQLDCMSRGEDVEQSLFLSVREGDSSVINCTYTDSSSTYLYWYK
QEPGAGLQLLTYIFSNMDMKQDQRLTVLLNKKDKHLSLRIADTQTGDSAIYFCAEKS
GYSGAGSYQLTFGKGTKLSVIPN
SEQ ID NO: 6
HLA-A2-restrcited CT83 TCR Beta chain variable domain (TRBV29-1-TRBJ1-1)
MLSLLLLLLGLGSVFSAVISQKPSRDICQRGTSLTIQCQVDSQVTMMFWYRQQPGQS
LTLIATANQGSEATYESGFVIDKFPISRPNLTFSTLTVSNMSPEDSSIYLCSVQDSEAFF
GQGTRLTVVE
SEQ ID NO: 7
21-nucleotide core of PD1 shRNA
CCGTGTCACACAACTGCCCAA
SEQ ID NO: 8
21-necleotide core of VHL shRNA
CAGGAGCGCATTGCACATCAA
SEQ ID NO: 9
21-necleotide core of PPP2R2D shRNA
AAGGTCATTACTCAGAATAAA
SEQ ID NO: 10
Human TCR Alpha constant domain (TRAC)
IQNPDPAVYQLRDSKSSDKSVCLFTDFDSQTNVSQSKDSDVYITDKTVLDMRSMDFK
SNSAVAWSNKSDFACANAFNNSIIPEDTFFPSPESSCDVKLVEKSFETDTNLNFQNLS
VIGFRILLLKVAGFNLLMTLRLWSS
SEQ ID NO: 11
Human TCR Beta constant domain 1 (TRBC1)
DLNKVFPPEVAVFEPSEAEISHTQKATLVCLATGFFPDHVELSWWVNGKEVHSGVST
DPQPLKEQPALNDSRYCLSSRLRVSATFWQNPRNHFRCQVQFYGLSENDEWTQDRA
KPVTQIVSAEAWGRADCGFTSVSYQQGVLSATILYEILLGKATLYAVLVSALVLMA
MVKRKDF
SEQ ID NO: 12
Human TCR Beta constant domain 2 (TRBC2)
DLKNVFPPEVAVFEPSEAEISHTQKATLVCLATGFYPDHVELSWWVNGKEVHSGVST
DPQPLKEQPALNDSRYCLSSRLRVSATFWQNPRNHFRCQVQFYGLSENDEWTQDRA
KPVTQIVSAEAWGRADCGFTSESYQQGVLSATILYEILLGKATLYAVLVSALVLMAM
VKRKDSRG
SEQ ID NO: 13
Murine TCR Alpha constant domain (trac)
IQNPEPAVYQLKDPRSQDSTLCLFTDFDSQINVPKTMESGTFITDKTVLDMKAMDSKS
NGAIAWSNQTSFTCQDIFKETNATYPSSDVPCDATLTEKSFETDMNLNFQNLSVMGL
RILLLKVAGFNLLMTLRLWSS
SEQ ID NO: 14
Murine TCR Beta constant domain 1 (trbc1)
DLRNVTPPKVSLFEPSKAEIANKQKATLVCLARGFFPDHVELSWWVNGKEVHSGVS
TDPQAYKESNYSYCLSSRLRVSATFWHNPRNHFRCQVQFHGLSEEDKWPEGSPKPV
TQNISAEAWGRADCGITSASYQQGVLSATILYEILLGKATLYAVLVSTLVVMAMVKR
KNS
SEQ ID NO: 15
Murine TCR Beta constant domain 2 (trbc2)
DLRNVTPPKVSLFEPSKAEIANKQKATLVCLARGFFPDHVELSWWVNGKEVHSGVS
TDPQAYKESNYSYCLSSRLRVSATFWHNPRNHFRCQVQFHGLSEEDKWPEGSPKPV
TQNISAEAWGRADCGITSASYHQGVLSATILYEILLGKATLYAVLVSGLVLMAMVK
KKNS
SEQ ID NO: 16
ZAP300
TSPDKPRPMPMDTSVYESPYSDPEELKDKKLFLKRDNLLIADIELGCGNFGSVRQGV
YRMRKKQIDVAIKVLKQGTEKADTEEMMREAQIMHQLDNPYIVRLIGVCQAEALML
VMEMAGGGPLHKFLVGKREEIPVSNVAELLHQVSMGMKYLEEKNFVHRDLAARNV
LLVNRHYAKISDFGLSKALGADDSYYTARSAGKWPLKWYAPECINFRKFSSRSDVW
SYGVTMWEALSYGQKPYKKMKGPEVMAFIEQGKRMECPPECPPELYALMSDCWIY
KWEDRPDFLTVEQRMRACYYSLASKVEGPPGSTQKAEAACA
SEQ ID NO: 17
ZAP327
DKKLFLKRDNLLIADIELGCGNFGSVRQGVYRMRKKQIDVAIKVLKQGTEKADTEE
MMREAQIMHQLDNPYIVRLIGVCQAEALMLVMEMAGGGPLHKFLVGKREEIPVSNV
AELLHQVSMGMKYLEEKNFVHRDLAARNVLLVNRHYAKISDFGLSKALGADDSYY
TARSAGKWPLKWYAPECINFRKFSSRSDVWSYGVTMWEALSYGQKPYKKMKGPEV
MAFIEQGKRMECPPECPPELYALMSDCWIYKWEDRPDFLTVEQRMRACYYSLASKV
EGPPGSTQKAEAACA
SEQ ID NO: 18
AAAAATCCCTCCCCGGCGGCGGCGGCGGCGGCGGCGGCGCCGGCGGTGGTGGCG
GCCCCGGGGCTGAGCGCTCGGCTGCAGCGGCGCGGAGGCCGTCTCCCTGGTCTG
CCGCGGTCCCCGCCCGTCCCGCCGCCGGCTGCCATGGCAGGAGCCGGAGGCGGC
GGCTGCCCCGCGGGCGGCAACGACTTCCAGTGGTGCTTCTCGCAGGTCAAGGGG
GCCATCGACGAGGACGTGGCCGAAGCGGACATCATTTCCACCGTTGAGTTTAATT
ACTCTGGAGATCTTCTTGCAACAGGAGACAAGGGCGGCAGAGTTGTTATTTTTCA
GCGTGAACAAGAGAATAAAAGCCGCCCTCATTCTAGGGGAGAATATAATGTTTA
CAGCACCTTTCAAAGTCATGAACCGGAGTTTGACTATTTGAAAAGTCTAGAAATT
GAGGAAAAAATTAATAAAATTAGGTGGTTACCACAACAGAATGCTGCTCATTTTC
TACTGTCTACAAATGATAAAACTATAAAATTATGGAAAATAAGTGAACGGGATA
AAAGAGCAGAAGGTTATAACCTGAAAGACGAAGATGGAAGACTTCGAGACCCAT
TTAGGATCACGGCGCTACGGGTCCCAATATTGAAGCCCATGGATCTTATGGTAGA
AGCGAGTCCACGGCGAATTTTTGCAAATGCTCACACATATCATATAAATTCCATT
TCAGTAAATAGTGATCATGAAACATATCTTTCTGCAGATGACCTGAGAATTAATT
TATGGCACTTAGAAATCACAGATAGAAGCTTTAACATCGTGGACATCAAGCCTGC
TAACATGGAGGAGCTGACCGAAGTCATCACTGCAGCCGAGTTCCACCCGCACCA
GTGCAACGTGTTCGTCTACAGCAGTAGCAAAGGGACCATCCGCCTGTGTGACATG
CGCTCCTCGGCCCTGTGCGACAGACACTCCAAGTTTTTTGAAGAGCCTGAAGATC
CCAGCAGTAGGTCCTTCTTCTCAGAAATAATTTCATCCATATCCGATGTAAAATT
CAGTCATAGTGGGCGGTACATGATGACCAGAGACTACCTGTCGGTGAAGGTGTG
GGACCTCAACATGGAGAGCAGGCCGGTGGAGACCCACCAGGTCCACGAGTACCT
GCGCAGCAAGCTCTGCTCTCTCTATGAGAACGACTGCATCTTTGACAAGTTTGAG
TGTTGCTGGAACGGTTCGGATAGCGCCATCATGACCGGGTCCTATAACAACTTCT
TCAGGATGTTTGATAGAGACACGCGGAGGGATGTGACCCTGGAGGCCTCGAGAG
AGAGCAGCAAACCGCGCGCCAGCCTCAAACCCCGGAAGGTGTGTACGGGGGGTA
AGCGGAGGAAAGACGAGATCAGTGTGGACAGTCTGGACTTCAACAAGAAGATCC
TGCACACAGCCTGGCACCCCGTGGACAATGTCATTGCCGTGGCTGCCACCAATAA
CTTGTACATATTCCAGGACAAAATCAACTAGAGACGCGAACGTGAGGACCAAGT
CTTGTCTTGCATAGTTAAGCCGGACATTTTTCTGTCAGAGAAAAGGCATCATTGT
CCGCTCCATTAAGAACAGTGACGCACCTGCTACTTCCCTTCACAGACACAGGAGA
AAGCCGCCTCCGCTGGAGGCCCGGTGTGGTTCCGCCTCGGCGAGGCGCGAGACA
GGCGCTGCTGCTCACGTGGAGACGCTCTCGAAGCAGAGTTGACGGACACTGCTC
CCAAAAGGTCATTACTCAGAATAAATGTATTTATTICAGTCCGAGCCTTCCTTTCC
AATTTATAGACCAAAAAATTAACATCCAAGAGAAAAGTTATTGTCAGATACCGC
TCTTTCTCCAACTTTCCCTCTTTCTCTGCCATCACACTTGGGCCTTCACTGCAGCGT
GGTGTGGCCACCGTCCGTGTCCTCTCGGCCTTCCTCCGAGTCCAGGTGGACTCTG
TGGATGTGTGGATGTGGCCCGAGCAGGCTCAGGCGGCCCCACTCACCCACAGCA
TCCGCCGCCACCCCTTCGGGTGTGAGCGCTCAATAAAAACAACACACTATAAAGT
GTTTTTAAATCCAAACAGAAGTATTGTCTTTTTATTTAATTTTATTGCATAGAATG
AAGTTATGCAGGGTTCTTCTTTGGAACTAACTGTTTGAGAAATGTGTGTCCTTCTT
TGGCAGCGTGGGGGTATGTGTGCAGCATTTCTGTCCCCAAGCTGCTGCCCGCTGT
GTTTCTGTAGAAGTAGCCCATCAGATACACCAGGTAAGGTCTGGGAAAAGCCAG
AACCGAGGCCACCTCTGAGAAAGAAAATTGCAAGGAAGAACCAAAATTGCTGCC
ATCCAGTAAGCCTGGTTTAAAGTAGCTTCAAATTCACTACAGTTGAAGAAATACG
TGCTTGCTTTCTAAGGCTTTTGAAAAAGCACTTTGGGGAAAGTATACTTTTTAAA
CATTGCTAAAATTACATGCATGCTAAATTTCATCCTGCATTTCAAGCCAGCAAAA
TTAAGCAATTTTAATTACACGATGCCACCAGTACGTTGGTTTATTTTCAAAGTAG
GATCTTTGATACCAGAAATCAAGATTTTCCAAGACAAATAATACAGAGCTGTACC
AATGCTGAGTGACCAGAGCTTGCTTCCGTGGGATTTAACACACGCCCACTACGTG
TCCCCGAGGGGAGTGGGGAGTCGGGCTCCCGTGCCCCTGTGGAGATGGAGGTGT
GTGCTGATCCCCCGTCCGCCTGTGGAGATGAAGGTGTGTGTTGATTCCTCCTGCT
GCTGTGGAGATGGAGGTGTGTGCTGATCGCCCGTCACCCTGTCTAGATGAAGGTA
TGTGCTGATTGCCTGTGCCCCTGTGGAGAAGGAGGTGTGTGCTGATCCCCTGTGC
CCTGTGGAGATGGAGGTGTGTGCTGCTCCCTCGTGCCCCTGTGGAGATGGAGGTG
TGTGCTGATCCCCCATCCCATCCCCCTGTCTAGATGAAGGTGTGTGCTGATTCCTC
ATGCCCCTGTCTGGATGAAGGTGTGTGCTGATCCCCCGTCCCCCTGTGGAGATGA
AGGTGTGTGCTGATCCCCCGTGCCCCTGTGGAGATGAAGGTGCGTGCTGATCCCC
CATCTCCCTGTGGAGATGAAGGTGTGTGCTGATCCCCATCCCCCTGTGGAGATGA
AGGGATATGCTGATCCCCTGTCCCACTGTGGAGATGAAGGGGTGTGCTGATCCCC
CGTCCCCCTGTGGAGATGAAGGTGTGTGCTGATCCCCCATCTCCCTGTGGAGATG
AAGGGGTGTGCCAATCCCCCGCGCCCCTGTGGAGATGGAGGCGTGTGCTGATCC
CCATCCCCCTGTGGAGATGAAGGTGTGTGCTGATCCCCCATCCCCATGTGGAGAT
GAAGGGGTGTGTTGATCCCCTGTCCCCCTGTGGAGATGGTGTGTGCTGATCCCCG
TCCCCCTGTGGAGATGAAGGTGTGTGCTGATCCCCCATCTCCCTGTGAAGATGAA
GGGGTGTGCTGATCCCCCGTGCCCCTGTGGACATGGAGGCGTGTGCTGATCCCCC
GTCCCCCTGTGGAGATGAAGGTGTGTGCTGATCCCCCATCCCCATGTGGAGATGA
AGGGGTGTGTTGATCCCCCGTCCCCCTGTGGAGATGAAGGTGTGTGCTGATCCCC
GTCCCCCTGTGGAGATGAAGGGGTGTGCTGATCCCCCATGCCCTGTGGAGATGGA
GGTGTGTGCTGATCCCTCGTGCCCCTGTGGAGATAGAGGTGTGTACTGATCCCCT
GTTCCCCTATCTAGATGAAGGTGTGTACTGCTGATCCCCCGTCCCCCTGTGGAGA
TGAAGGTGTGTGCTGATCCCCCATCCCCCTGTGGAGATGAAGGGGTGTGCTGATC
CCCCGTCTTCCTGTGGAGATGAAGGTGTGTGCTGATTCCTCATGCCCCTGTGGAG
ATGGAGGTGTGTGCTGATCCCCCATCCCCCTGTGGGGATGAAGGTGTGTGCTGAT
CCCCCATCCCCCTGTGGAGATGAAGGGGTATGCTGATCCCCCGTCCCCCTGTGGA
GATGAAGGTGTGTGCTGATCCCCCATCCCCCTGTGGAGATGAAGGGGTATGCTGA
TCCCCTGTCCCCCTGTGGGGATGAAGGTGTGTGCTGATCTCTTGTCCCCCTGTGGA
GATGGAGGTGTGTGCTGATCCCCCATCCCCCTGTGGAGATGAAGATGTGTGCTGA
TCCCCCGTCCTCCTGTGGAGATGAAGGTGTGTGCTGCTTCCTCGTGCCCCTGTGG
AGATGGAGGTGTGTGCTGGTCCCCCGTCCCCCTGTGGAGATGGAGGTGTGTGCTG
ATCCCCCGTCCCCCTGTGGAGATGGAGGCGTGTGCTGATCCCCCATCCCCCTGTG
GAGATGAAGGCGTGTGCTGATCCGCCATCCCACTGTGGAGATGAAGGCGTGTGC
TGATCCCCCATCCCCCTGTGGAGATAAAGGTGTGTGCTGATCCCCCATCCCCCTG
TGGAGATGAAGGCGTGTGCTGATCCGCCATCCCACTGTGGAGATGAAGGCGTGT
GCTGATCCCCCATCCCCCTGTGGAGATGAAGGCGTGTGCTCATCCCCCATCCCCC
TGTGGAGATGAAGGCGTGTGCTGATCCGCCATCCCACTGTGGAGATGAAGGTGT
GTGCTGATCCCCCATCCCCCTGTGGAGATGAAGGTGTGTGCTGATCCCCGGTCCC
CCTGTGGAGATGAAGGTGTGTGCTGATCCCCCGTCCTCCTGTGGAGATGAAGGCG
TGTGCTGATCCCCCGTCCCCCTGTGGAGATGAAGGCGTGTGCTGATCCGCCGTCC
CCCTGTGGAGATGAAGGCGTGTGCTGATCCGCCATCCCCCTGTGGAGATGAAGG
CGTGTGCTCATCCCCCATCCCCCTGTGGAGATGAAGGCGTGTGCTGATCCGCCAT
CCCACTGTGGAGATGAAGGTGTGTGCTGATCCCCCATCCCCCTGTGGAGATGAAG
GCGTGTGCTGATCCCCGGTCCCCCTGTGGAGATGAAGGTGTGTGTTGATCCCCCA
TCCCCCTGTGGAGGTGAAGGTGTGTGCTGATTACTAATCCACATGGGCAAGAACA
GGATGTCCGTCATTACCCTGAATCAATGCTAACACAGTGCCACTTGAGTTCACAT
TAAGTTAGAAATTGAGAATCTAAAGGTACCTTTATTTTAACTAAAAAATAATTTA
TATACTGTATATTGATTGTGACACAATTTACAAAGTCTGAGGTGTGGAACAGTTA
TTTAAGCATTAGTCAACCCTGGTCCTTAAGACAGTTCTAGTAAAATGGGATTGTA
TATATTTGTTCAACTATTTTGACCAAAAAGTTCAATAAATTTTAAATGTTTAACTG
GA
SEQ ID NO: 19
AAAAATCCCTCCCCGGCGGCGGCGGCGGCGGCGGCGGCGCCGGCGGTGGTGGCG
GCCCCGGGGCTGAGCGCTCGGCTGCAGCGGCGCGGAGGCCGTCTCCCTGGTCTG
CCGCGGTCCCCGCCCGTCCCGCCGCCGGCTGCCATGGCAGGAGCCGGAGGCGGC
GGCTGCCCCGCGGGCGGCAACGACTTCCAGTGGTGCTTCTCGCAGGTCAAGGGG
GCCATCGACGAGGACGTGGCCGAAGGGCGTGGAGAGGAGAGCTCGGGGCTGCT
GCCACAGATCATGCTGAGCCGCCAGCAGCTCCACCTGCCCTTGGCTTTGCTCATC
CGTGGAAATGACAACAGCGTGAAAACGGCAGATGATGTACAAGGATTATGATGA
AAATAGTCTTGACCTCACGGACCCTCTGAGAGGTTCTTGACCGTTGCACCCTCAA
GGGCCCACAGACCGCACTTTGAGAACTGCTGCTTGAATGCAAGCTATTAGCAGTA
GGAGTGTCAAGGACAGGGCAGACTTCAGGATTAACTGTTCCTTCTCATCTTTACC
AGATTGTACTGGTTTTGTCTCAAAATACAAAAATAGAGTGTTTATTATGTAAAGA
AAATCAAATGATAGAACAGCTGAGAACAGCTGGATCAAGAGCACAGAGCGGAA
GTTCCTCTTCATCCCACTGCTGTCCTGGAGTCTTCCTCAGGACCCACTGCTCCTAA
CTGTATCTTTGTAGATCATTTTCTATGTGTTTATTTATTTATATTTTAAAACCTAGT
TTGTGTATAAGCAATCTTTTTTAATCCACAAATGTGGAATCTTATTCTAGACTTTG
AATAAGATTTTCTGTATTAGTCAGTATTGTTTTTTGCTACTTCTTTTCCTTTCCTTT
CCTTTTTTGGAAATAGGGTCTTACTCCTCTGTCACCCAGGCTGGAGTGCAGTGGT
GCGATCAGAGCTCACTGCAGCCTGATCAGGCTCAAGCAATCCTCTCCCTGCTCAG
CCTCCTGAGTAGCTGGAACTATAGGCTTGCACTGCCATGCCCAGCTTTTTTCTATT
TTTTGTAGAGACAGGGTTTTGCTGTGTTGCCCAGGATGGTCTTGAACTCCTGGAC
TCAAGTGATCCTCCCACCTCGGCCTCCCAAAATGCTGGGAATACAAGCATGAGCC
ATGGCATGTGGCCGCCACTTGCCTTTTTTCATTAAGAGTCTATAATTGGAGATCTT
ACAGGGAACTGGGCTTTCTGCTCAGCCATACTCGAGCTCATTTGACCCCCGGCCA
CTGCCCCTTGAAATCGGACATCATTTCCACCGTTGAGTTTAATTACTCTGGAGATC
TTCTTGCAACAGGAGACAAGGGCGGCAGAGTTGTTATTTTTCAGCGTGAACAAG
AGAATAAAAGCCGCCCTCATTCTAGGGGAGAATATAATGTTTACAGCACCTTTCA
AAGTCATGAACCGGAGTTTGACTATTTGAAAAGTCTAGAAATTGAGGAAAAAAT
TAATAAAATTAGGTGGTTACCACAACAGAATGCTGCTCATTTTCTACTGTCTACA
AATGATAAAACTATAAAATTATGGAAAATAAGTGAACGGGATAAAAGAGCAGA
AGGTTATAACCTGAAAGACGAAGATGGAAGACTTCGAGACCCATTTAGGATCAC
GGCGCTACGGGTCCCAATATTGAAGCCCATGGATCTTATGGTAGAAGCGAGTCC
ACGGCGAATTTTTGCAAATGCTCACACATATCATATAAATTCCATTTCAGTAAAT
AGTGATCATGAAACATATCTTTCTGCAGATGACCTGAGAATTAATTTATGGCACT
TAGAAATCACAGATAGAAGCTTTAACATCGTGGACATCAAGCCTGCTAACATGG
AGGAGCTGACCGAAGTCATCACTGCAGCCGAGTTCCACCCGCACCAGTGCAACG
TGTTCGTCTACAGCAGTAGCAAAGGGACCATCCGCCTGTGTGACATGCGCTCCTC
GGCCCTGTGCGACAGACACTCCAAGTTTTTTGAAGAGCCTGAAGATCCCAGCAGT
AGGTCCTTCTTCTCAGAAATAATTTCATCCATATCCGATGTAAAATTCAGTCATA
GTGGGCGGTACATGATGACCAGAGACTACCTGTCGGTGAAGGTGTGGGACCTCA
ACATGGAGAGCAGGCCGGTGGAGACCCACCAGGTCCACGAGTACCTGCGCAGCA
AGCTCTGCTCTCTCTATGAGAACGACTGCATCTTTGACAAGTTTGAGTGTTGCTG
GAACGGTTCGGATAGCGCCATCATGACCGGGTCCTATAACAACTTCTTCAGGATG
TTTGATAGAGACACGCGGAGGGATGTGACCCTGGAGGCCTCGAGAGAGAGCAGC
AAACCGCGCGCCAGCCTCAAACCCCGGAAGGTGTGTACGGGGGGTAAGCGGAGG
AAAGACGAGATCAGTGTGGACAGTCTGGACTTCAACAAGAAGATCCTGCACACA
GCCTGGCACCCCGTGGACAATGTCATTGCCGTGGCTGCCACCAATAACTTGTACA
TATTCCAGGACAAAATCAACTAGAGACGCGAACGTGAGGACCAAGTCTTGTCTT
GCATAGTTAAGCCGGACATTTTTCTGTCAGAGAAAAGGCATCATTGTCCGCTCCA
TTAAGAACAGTGACGCACCTGCTACTTCCCTTCACAGACACAGGAGAAAGCCGC
CTCCGCTGGAGGCCCGGTGTGGTTCCGCCTCGGCGAGGCGCGAGACAGGCGCTG
CTGCTCACGTGGAGACGCTCTCGAAGCAGAGTTGACGGACACTGCTCCCAAAAG
GTCATTACTCAGAATAAATGTATTTATTICAGTCCGAGCCTTCCTTTCCAATTTAT
AGACCAAAAAATTAACATCCAAGAGAAAAGTTATTGTCAGATACCGCTCTTTCTC
CAACTTTCCCTCTTTCTCTGCCATCACACTTGGGCCTTCACTGCAGCGTGGTGTGG
CCACCGTCCGTGTCCTCTCGGCCTTCCTCCGAGTCCAGGTGGACTCTGTGGATGT
GTGGATGTGGCCCGAGCAGGCTCAGGCGGCCCCACTCACCCACAGCATCCGCCG
CCACCCCTTCGGGTGTGAGCGCTCAATAAAAACAACACACTATAAAGTGTTTTTA
AATCCAAACAGAAGTATTGTCTTTTTATTTAATTTTATTGCATAGAATGAAGTTAT
GCAGGGTTCTTCTTTGGAACTAACTGTTTGAGAAATGTGTGTCCTTCTTTGGCAGC
GTGGGGGTATGTGTGCAGCATTTCTGTCCCCAAGCTGCTGCCCGCTGTGTTTCTGT
AGAAGTAGCCCATCAGATACACCAGGTAAGGTCTGGGAAAAGCCAGAACCGAG
GCCACCTCTGAGAAAGAAAATTGCAAGGAAGAACCAAAATTGCTGCCATCCAGT
AAGCCTGGTTTAAAGTAGCTTCAAATTCACTACAGTTGAAGAAATACGTGCTTGC
TTTCTAAGGCTTTTGAAAAAGCACTTTGGGGAAAGTATACTTTTTAAACATTGCT
AAAATTACATGCATGCTAAATTTCATCCTGCATTTCAAGCCAGCAAAATTAAGCA
ATTTTAATTACACGATGCCACCAGTACGTTGGTTTATTTTCAAAGTAGGATCTTTG
ATACCAGAAATCAAGATTTTCCAAGACAAATAATACAGAGCTGTACCAATGCTG
AGTGACCAGAGCTTGCTTCCGTGGGATTTAACACACGCCCACTACGTGTCCCCGA
GGGGAGTGGGGAGTCGGGCTCCCGTGCCCCTGTGGAGATGGAGGTGTGTGCTGA
TCCCCCGTCCGCCTGTGGAGATGAAGGTGTGTGTTGATTCCTCCTGCTGCTGTGG
AGATGGAGGTGTGTGCTGATCGCCCGTCACCCTGTCTAGATGAAGGTATGTGCTG
ATTGCCTGTGCCCCTGTGGAGAAGGAGGTGTGTGCTGATCCCCTGTGCCCTGTGG
AGATGGAGGTGTGTGCTGCTCCCTCGTGCCCCTGTGGAGATGGAGGTGTGTGCTG
ATCCCCCATCCCATCCCCCTGTCTAGATGAAGGTGTGTGCTGATTCCTCATGCCCC
TGTCTGGATGAAGGTGTGTGCTGATCCCCCGTCCCCCTGTGGAGATGAAGGTGTG
TGCTGATCCCCCGTGCCCCTGTGGAGATGAAGGTGCGTGCTGATCCCCCATCTCC
CTGTGGAGATGAAGGTGTGTGCTGATCCCCATCCCCCTGTGGAGATGAAGGGAT
ATGCTGATCCCCTGTCCCACTGTGGAGATGAAGGGGTGTGCTGATCCCCCGTCCC
CCTGTGGAGATGAAGGTGTGTGCTGATCCCCCATCTCCCTGTGGAGATGAAGGGG
TGTGCCAATCCCCCGCGCCCCTGTGGAGATGGAGGCGTGTGCTGATCCCCATCCC
CCTGTGGAGATGAAGGTGTGTGCTGATCCCCCATCCCCATGTGGAGATGAAGGG
GTGTGTTGATCCCCTGTCCCCCTGTGGAGATGGTGTGTGCTGATCCCCGTCCCCCT
GTGGAGATGAAGGTGTGTGCTGATCCCCCATCTCCCTGTGAAGATGAAGGGGTGT
GCTGATCCCCCGTGCCCCTGTGGACATGGAGGCGTGTGCTGATCCCCCGTCCCCC
TGTGGAGATGAAGGTGTGTGCTGATCCCCCATCCCCATGTGGAGATGAAGGGGT
GTGTTGATCCCCCGTCCCCCTGTGGAGATGAAGGTGTGTGCTGATCCCCGTCCCC
CTGTGGAGATGAAGGGGTGTGCTGATCCCCCATGCCCTGTGGAGATGGAGGTGT
GTGCTGATCCCTCGTGCCCCTGTGGAGATAGAGGTGTGTACTGATCCCCTGTTCC
CCTATCTAGATGAAGGTGTGTACTGCTGATCCCCCGTCCCCCTGTGGAGATGAAG
GTGTGTGCTGATCCCCCATCCCCCTGTGGAGATGAAGGGGTGTGCTGATCCCCCG
TCTTCCTGTGGAGATGAAGGTGTGTGCTGATTCCTCATGCCCCTGTGGAGATGGA
GGTGTGTGCTGATCCCCCATCCCCCTGTGGGGATGAAGGTGTGTGCTGATCCCCC
ATCCCCCTGTGGAGATGAAGGGGTATGCTGATCCCCCGTCCCCCTGTGGAGATGA
AGGTGTGTGCTGATCCCCCATCCCCCTGTGGAGATGAAGGGGTATGCTGATCCCC
TGTCCCCCTGTGGGGATGAAGGTGTGTGCTGATCTCTTGTCCCCCTGTGGAGATG
GAGGTGTGTGCTGATCCCCCATCCCCCTGTGGAGATGAAGATGTGTGCTGATCCC
CCGTCCTCCTGTGGAGATGAAGGTGTGTGCTGCTTCCTCGTGCCCCTGTGGAGAT
GGAGGTGTGTGCTGGTCCCCCGTCCCCCTGTGGAGATGGAGGTGTGTGCTGATCC
CCCGTCCCCCTGTGGAGATGGAGGCGTGTGCTGATCCCCCATCCCCCTGTGGAGA
TGAAGGCGTGTGCTGATCCGCCATCCCACTGTGGAGATGAAGGCGTGTGCTGATC
CCCCATCCCCCTGTGGAGATAAAGGTGTGTGCTGATCCCCCATCCCCCTGTGGAG
ATGAAGGCGTGTGCTGATCCGCCATCCCACTGTGGAGATGAAGGCGTGTGCTGAT
CCCCCATCCCCCTGTGGAGATGAAGGCGTGTGCTCATCCCCCATCCCCCTGTGGA
GATGAAGGCGTGTGCTGATCCGCCATCCCACTGTGGAGATGAAGGTGTGTGCTG
ATCCCCCATCCCCCTGTGGAGATGAAGGTGTGTGCTGATCCCCGGTCCCCCTGTG
GAGATGAAGGTGTGTGCTGATCCCCCGTCCTCCTGTGGAGATGAAGGCGTGTGCT
GATCCCCCGTCCCCCTGTGGAGATGAAGGCGTGTGCTGATCCGCCGTCCCCCTGT
GGAGATGAAGGCGTGTGCTGATCCGCCATCCCCCTGTGGAGATGAAGGCGTGTG
CTCATCCCCCATCCCCCTGTGGAGATGAAGGCGTGTGCTGATCCGCCATCCCACT
GTGGAGATGAAGGTGTGTGCTGATCCCCCATCCCCCTGTGGAGATGAAGGCGTGT
GCTGATCCCCGGTCCCCCTGTGGAGATGAAGGTGTGTGTTGATCCCCCATCCCCC
TGTGGAGGTGAAGGTGTGTGCTGATTACTAATCCACATGGGCAAGAACAGGATG
TCCGTCATTACCCTGAATCAATGCTAACACAGTGCCACTTGAGTTCACATTAAGT
TAGAAATTGAGAATCTAAAGGTACCTTTATTTTAACTAAAAAATAATTTATATAC
TGTATATTGATTGTGACACAATTTACAAAGTCTGAGGTGTGGAACAGTTATTTAA
GCATTAGTCAACCCTGGTCCTTAAGACAGTTCTAGTAAAATGGGATTGTATATAT
TTGTTCAACTATTTTGACCAAAAAGTTCAATAAATTTTAAATGTTTAACTGGA
SEQ ID NO: 20
HLA-A2-restrcited CT83 TCR Alpha chain variable domain fused with murine Alpha
constant domain
MKTFAGFSFLFLWLQLDCMSRGEDVEQSLFLSVREGDSSVINCTYTDSSSTYLYWYK
QEPGAGLQLLTYIFSNMDMKQDQRLTVLLNKKDKHLSLRIADTQTGDSAIYFCAEKS
GYSGAGSYQLTFGKGTKLSVIPNIQNPEPAVYQLKDPRSQDSTLCLFTDFDSQINVPK
TMESGTFITDKTVLDMKAMDSKSNGAIAWSNQTSFTCQDIFKETNATYPSSDVPCDA
TLTEKSFETDMNLNFQNLSVMGLRILLLKVAGFNLLMTLRLWSS
SEQ ID NO: 21
HLA-A2-restrcited CT83 TCR Beta chain variable domain fused with murine Beta constant
domain 2
MLSLLLLLLGLGSVFSAVISQKPSRDICQRGTSLTIQCQVDSQVTMMFWYRQQPGQS
LTLIATANQGSEATYESGFVIDKFPISRPNLTFSTLTVSNMSPEDSSIYLCSVQDSEAFF
GQGTRLTVVEDLRNVTPPKVSLFEPSKAEIANKQKATLVCLARGFFPDHVELSWWV
NGKEVHSGVSTDPQAYKESNYSYCLSSRLRVSATFWHNPRNHFRCQVQFHGLSEED
KWPEGSPKPVTQNISAEAWGRADCGITSASYHQGVLSATILYEILLGKATLYAVLVS
GLVLMAMVKKKNS
SEQ ID NO: 22
HLA-A2-restricted NY-ESO-1 TCR (S2) Alpha chain variable domain fused with murine
Alpha constant domain
METLLGLLILWLQLQWVSSKQEVTQIPAALSVPEGENLVLNCSFTDSAIYNLQWFRQ
DPGKGLTSLLLIQSSQREQTSGRLNASLDKSSGRSTLYIAASQPGDSATYLCAVRPQT
GGSYIPTFGRGTSLIVHPYIQNPEPAVYQLKDPRSQDSTLCLFTDFDSQINVPKTMESG
TFITDKTVLDMKAMDSKSNGAIAWSNQTSFTCQDIFKETNATYPSSDVPCDATLTEK
SFETDMNLNFQNLSVMGLRILLLKVAGFNLLMTLRLWSS
SEQ ID NO: 23
HLA-A2-restricted NY-ESO-1 TCR (S2) (G50A, A51E) Beta chain variable domain fused
with murine Beta constant domain 2
MAPRLLCCAALSLLWAGPVNAGVTQTPKFQVLKTGQSMTLQCAQDMNHEYMSWY
RQDPGMGLRLIHYSVAEGITDQGEVPNGYNVSRSTTEDFPLRLLSAAPSQTSVYFCAS
SYVGAAGELFFGEGSRLTVLEDLRNVTPPKVSLFEPSKAEIANKQKATLVCLARGFFP
DHVELSWWVNGKEVHSGVSTDPQAYKESNYSYCLSSRLRVSATFWHNPRNHFRCQ
VQFHGLSEEDKWPEGSPKPVTQNISAEAWGRADCGITSASYHQGVLSATILYEILLGK
ATLYAVLVSGLVLMAMVKKKNS
SEQ ID NO: 24
HLA-A2-restricted NY-ESO-1 TCR (S5) Alpha chain variable domain fused with murine
Alpha constant domain
METLLGLLILWLQLQWVSSKQEVTQIPAALSVPEGENLVLNCSFTDSAIYNLQWFRQ
DPGKGLTSLLLIQSSQREQTSGRLNASLDKSSGRSTLYIAASQPGDSATYLCAVRPQT
GGSYIPTFGRGTSLIVHPYIQNPEPAVYQLKDPRSQDSTLCLFTDFDSQINVPKTMESG
TFITDKTVLDMKAMDSKSNGAIAWSNQTSFTCQDIFKETNATYPSSDVPCDATLTEK
SFETDMNLNFQNLSVMGLRILLLKVAGFNLLMTLRLWSS
SEQ ID NO: 25
HLA-A2-restricted NY-ESO-1 TCR (S5) (G50A, A51E, A97L) Beta chain variable domain
fused with murine Beta constant domain 2
MAPRLLCCAALSLLWAGPVNAGVTQTPKFQVLKTGQSMTLQCAQDMNHEYMSWY
RQDPGMGLRLIHYSVAEGITDQGEVPNGYNVSRSTTEDFPLRLLSAAPSQTSVYFCAS
SYVGLAGELFFGEGSRLTVLEDLRNVTPPKVSLFEPSKAEIANKQKATLVCLARGFFP
DHVELSWWVNGKEVHSGVSTDPQAYKESNYSYCLSSRLRVSATFWHNPRNHFRCQ
VQFHGLSEEDKWPEGSPKPVTQNISAEAWGRADCGITSASYHQGVLSATILYEILLGK
ATLYAVLVSGLVLMAMVKKKNS
SEQ ID NO: 26
HLA-A2-restricted pp65 epitope (495-503)
NLVPMVATV
SEQ ID NO: 27
HLA-A2-restricted pp65 TCR (#1-15) Alpha chain variable domain (TRAV21-TRAJ18)
METLLGLLILWLQLQWVSSKQEVTQIPAALSVPEGENLVLNCSFTDSAIYNLQWFRQ
DPGKGLTSLLLIQSSQREQTSGRLNASLDKSSGRSTLYIAASQPGDSATYLCAVRPQG
STLGRLYFGRGTQLTVWPD
SEQ ID NO: 28
HLA-A2-restricted pp65 TCR (#1-15) Beta chain variable domain (TRBV13-TRBJ1-5)
MLSPDLPDSAWNTRLLCRVMLCLLGAGSVAAGVIQSPRHLIKEKRETATLKCYPIPR
HDTVYWYQQGPGQDPQFLISFYEKMQSDKGSIPDRFSAQQFSGYHSELNMSSLELGD
SALYFCASSLENNQPQHFGDGTRLSILE
Seq ID NO: 29
HLA-A2-restricted pp65 TCR (#132-3) Alpha chain variable domain (TRAV24-TRAJ49)
MEKNPLAAPLLILWFHLDCVSSILNVEQSPQSLHVQEGDSTNFTCSFPSSNFYALHWY
RWETAKSPEALFVMTLNGDEKKKGRISATLNTKEGYSYLYIKGSQPEDSATYLCARN
TGNQFYFGTGTSLTVIPN
SEQ ID NO: 30
HLA-A2-restricted pp65 TCR (#132-3) Beta chain variable domain (TRBV6-5-TRBJ1-2)
MSIGLLCCAALSLLWAGPVNAGVTQTPKFQVLKTGQSMTLQCAQDMNHEYMSWY
RQDPGMGLRLIHYSVGAGITDQGEVPNGYNVSRSTTEDFPLRLLSAAPSQTSVYFCAS
SPITGTGDYGYTFGSGTRLTVVE
SEQ ID NO: 31
HLA-A2-restriced IE-1 epitope (316-324)
VLEETSVML
SEQ ID NO: 32
HLA-A2-restricted IE-1 TCR Alpha chain variable domain (TRAV25-TRAJ42)
MLLITSMLVLWMQLSQVNGQQVMQIPQYQHVQEGEDFTTYCNSSTTLSNIQWYKQ
RPGGHPVFLIQLVKSGEVKKQKRLTFQFGEAKKNSSLHITATQTTDVGTYFCAGHIY
GGSQGNLIFGKGTKLSVKPN
SEQ ID NO: 33
HLA-A2-restricted IE-1 TCR Beta chain variable domain (TRBV5-1-TRBJ2-5)
MGSRLLCWVLLCLLGAGPVKAGVTQTPRYLIKTRGQQVTLSCSPISGHRSVSWYQQ
TPGQGLQFLFEYFSETQRNKGNFPGRFSGRQFSNSRSEMNVSTLELGDSALYLCASSH
HQGPLETQYFGPGTRLLVLE
SEQ ID NO:34
NY-ESO-1 PEP161-180
WITQCFLPVFLAQPPSGQRR
SEQ ID NO:35
NY-ESO-1 PEP156-175
LSLLMWITQCFLPVFLAQPP
SEQ ID NO:36
CT83 PEP6-14
LLASSILCA
SEQ ID NO:37
CT83 PEP4-12
YLLLASSIL
SEQ ID NO:38
CT83 PEP79-87
RILVNLSMV
SEQ ID NO:39
CT83 PEP10-31
SILCALIVFWKYRRFQRNTGEM
SEQ ID NO:40
CT83 PEP66-76
ILNNFPHSIAR
SEQ ID NO: 41
DNA coding of HLA-DP4-redtricted NY-ESO-1 TCR Alpha chain variable domain
ATGGAGACTGTTCTGCAAGTACTCCTAGGGATATTGGGGTTCCAAGCAGCCTGGG
TCAGTAGCCAAGAACTGGAGCAGAGTCCTCAGTCCTTGATCGTCCAAGAGGGAA
AGAATCTCACCATAAACTGCACGTCATCAAAGACGTTATATGGCTTATACTGGTA
TAAGCAAAAGTATGGTGAAGGTCTTATCTTCTTGATGATGCTACAGAAAGGTGGG
GAAGAGAAAAGTCATGAAAAGATAACTGCCAAGTTGGATGAGAAAAAGCAGCA
AAGTTCCCTGCATATCACAGCCTCCCAGCCCAGCCATGCAGGCATCTACCTCTGT
GGAGCAGACATAGTAGACTATGGTCAGAATTTTGTCTTTGGTCCCGGAACCAGGT
TGTCCGTGCTGCCCTAT
SEQ ID NO: 42
DNA coding of HLA-DP4-redtricted NY-ESO-1 TCR Beta chain variable domain
ATGCTCTGCTCTCTCCTTGCCCTTCTCCTGGGCACTTTCTTTGGGGTCAGATCTCA
GACTATTCATCAATGGCCAGCGACCCTGGTGCAGCCTGTGGGCAGCCCGCTCTCT
CTGGAGTGCACTGTGGAGGGAACATCAAACCCCAACCTATACTGGTACCGACAG
GCTGCAGGCAGGGGCCTCCAGCTGCTCTTCTACTCCGTTGGTATTGGCCAGATCA
GCTCTGAGGTGCCCCAGAATCTCTCAGCCTCCAGACCCCAGGACCGGCAGTTCAT
CCTGAGTTCTAAGAAGCTCCTTCTCAGTGACTCTGGCTTCTATCTCTGTGCCTGGA
GGCGCCGGGGTTACGAGCAGTACTTCGGGCCGGGCACCAGGCTCACGGTCACAG
AG
SEQ ID NO: 43
DNA coding of HLA-A2-restrcited CT83 TCR Alpha chain variable domain
ATGAAGACATTTGCTGGATTTTCGTTCCTGTTTTTGTGGCTGCAGCTGGACTGTAT
GAGTAGAGGAGAGGATGTGGAGCAGAGTCTTTTCCTGAGTGTCCGAGAGGGAGA
CAGCTCCGTTATAAACTGCACTTACACAGACAGCTCCTCCACCTACTTATACTGG
TATAAGCAAGAACCTGGAGCAGGTCTCCAGTTGCTGACGTATATTTTTTCAAATA
TGGACATGAAACAAGACCAAAGACTCACTGTTCTATTGAATAAAAAGGATAAAC
ATCTGTCTCTGCGCATTGCAGACACCCAGACTGGGGACTCAGCTATCTACTTCTG
TGCAGAGAAGAGCGGGTACTCTGGGGCTGGGAGTTACCAACTCACTTTCGGGAA
GGGGACCAAACTCTCGGTCATACCAAAT
SEQ ID NO: 44
DNA coding of HLA-A2-restrcited CT83 TCR Beta chain variable domain
ATGCTGAGTCTTCTGCTCCTTCTCCTGGGACTAGGCTCTGTGTTCAGTGCTGTCAT
CTCTCAAAAGCCAAGCAGGGATATCTGTCAACGTGGAACCTCCCTGACGATCCA
GTGTCAAGTCGATAGCCAAGTCACCATGATGTTCTGGTACCGTCAGCAACCTGGA
CAGAGCCTGACACTGATCGCAACTGCAAATCAGGGCTCTGAGGCCACATATGAG
AGTGGATTTGTCATTGACAAGTTTCCCATCAGCCGCCCAAACCTAACATTCTCAA
CTCTGACTGTGAGCAACATGAGCCCTGAAGACAGCAGCATATATCTCTGCAGCGT
TCAAGACAGTGAAGCTTTCTTTGGACAAGGCACCAGACTCACAGTTGTAGAG
SEQ ID NO: 45
DNA coding of HLA-A2-restricted pp65 TCR (#1-15) Alpha chain variable domain
ATGGAGACCCTCTTGGGCCTGCTTATCCTTTGGCTGCAGCTGCAATGGGTGAGCA
GCAAACAGGAGGTGACGCAGATTCCTGCAGCTCTGAGTGTCCCAGAAGGAGAAA
ACTTGGTTCTCAACTGCAGTTTCACTGATAGCGCTATTTACAACCTCCAGTGGTTT
AGGCAGGACCCTGGGAAAGGTCTCACATCTCTGTTGCTTATTCAGTCAAGTCAGA
GAGAGCAAACAAGTGGAAGACTTAATGCCTCGCTGGATAAATCATCAGGACGTA
GTACTTTATACATTGCAGCTTCTCAGCCTGGTGACTCAGCCACCTACCTCTGTGCT
GTGAGGCCTCAGGGCTCAACCCTGGGGAGGCTATACTTTGGAAGAGGAACTCAG
TTGACTGTCTGGCCTGAT
SEQ ID NO: 46
DNA coding of HLA-A2-restricted pp65 TCR (#1-15) Beta chain variable domain
ATGCTTAGTCCTGACCTGCCTGACTCTGCCTGGAACACCAGGCTCCTCTGCCGTG
TCATGCTTTGTCTCCTGGGAGCAGGTTCAGTGGCTGCTGGAGTCATCCAGTCCCC
AAGACATCTGATCAAAGAAAAGAGGGAAACAGCCACTCTGAAATGCTATCCTAT
CCCTAGACACGACACTGTCTACTGGTACCAGCAGGGTCCAGGTCAGGACCCCCA
GTTCCTCATTTCGTTTTATGAAAAGATGCAGAGCGATAAAGGAAGCATCCCTGAT
CGATTCTCAGCTCAACAGTTCAGTGGCTATCATTCTGAACTGAACATGAGCTCCT
TGGAGCTGGGGGACTCAGCCCTGTACTTCTGTGCCAGCAGCTTAGAGAACAATCA
GCCCCAGCATTTTGGTGATGGGACTCGACTCTCCATCCTAGAG
SEQ ID NO: 47
DNA coding of HLA-A2-restricted pp65 TCR (#132-3) Alpha chain variable domain
ATGGAGAAGAATCCTTTGGCAGCCCCATTACTAATCCTCTGGTTTCATCTTGACT
GCGTGAGCAGCATACTGAACGTGGAACAAAGTCCTCAGTCACTGCATGTTCAGG
AGGGAGACAGCACCAATTTCACCTGCAGCTTCCCTTCCAGCAATTTTTATGCCTT
ACACTGGTACAGATGGGAAACTGCAAAAAGCCCCGAGGCCTTGTTTGTAATGAC
TTTAAATGGGGATGAAAAGAAGAAAGGACGAATAAGTGCCACTCTTAATACCAA
GGAGGGTTACAGCTATTTGTACATCAAAGGATCCCAGCCTGAAGACTCAGCCAC
ATACCTCTGTGCCCGAAACACCGGTAACCAGTTCTATTTTGGGACAGGGACAAGT
TTGACGGTCATTCCAAAT
SEQ ID NO: 48
DNA coding of HLA-A2-restricted pp65 TCR (#132-3) Beta chain variable domain
ATGAGCATCGGCCTCCTGTGCTGTGCAGCCTTGTCTCTCCTGTGGGCAGGTCCAG
TGAATGCTGGTGTCACTCAGACCCCAAAATTCCAGGTCCTGAAGACAGGACAGA
GCATGACACTGCAGTGTGCCCAGGATATGAACCATGAATACATGTCCTGGTATCG
ACAAGACCCAGGCATGGGGCTGAGGCTGATTCATTACTCAGTTGGTGCTGGTATC
ACTGACCAAGGAGAAGTCCCCAATGGCTACAATGTCTCCAGATCAACCACAGAG
GATTTCCCGCTCAGGCTGCTGTCGGCTGCTCCCTCCCAGACATCTGTGTACTTCTG
TGCCAGCAGTCCTATCACCGGGACAGGGGACTATGGCTACACCTTCGGTTCGGGG
ACCAGGTTAACCGTTGTAGAG
SEQ ID NO: 49
DNA coding of HLA-A2-restricted IE-1 TCR Alpha chain variable domain
ATGCTACTCATCACATCAATGTTGGTCTTATGGATGCAATTGTCACAGGTGAATG
GACAACAGGTAATGCAAATTCCTCAGTACCAGCATGTACAAGAAGGAGAGGACT
TCACCACGTACTGCAATTCCTCAACTACTTTAAGCAATATACAGTGGTATAAGCA
AAGGCCTGGTGGACATCCCGTTTTTTTGATACAGTTAGTGAAGAGTGGAGAAGTG
AAGAAGCAGAAAAGACTGACATTTCAGTTTGGAGAAGCAAAAAAGAACAGCTCC
CTGCACATCACAGCCACCCAGACTACAGATGTAGGAACCTACTTCTGTGCAGGAC
ACATTTATGGAGGAAGCCAAGGAAATCTCATCTTTGGAAAAGGCACTAAACTCT
CTGTTAAACCAAAT
SEQ ID NO: 50
DNA coding of HLA-A2-restricted IE-1 TCR Beta chain variable domain
ATGGGCTCCAGGCTGCTCTGTTGGGTGCTGCTTTGTCTCCTGGGAGCAGGCCCAG
TAAAGGCTGGAGTCACTCAAACTCCAAGATATCTGATCAAAACGAGAGGACAGC
AAGTGACACTGAGCTGCTCCCCTATCTCTGGGCATAGGAGTGTATCCTGGTACCA
ACAGACCCCAGGACAGGGCCTTCAGTTCCTCTTTGAATACTTCAGTGAGACACAG
AGAAACAAAGGAAACTTCCCTGGTCGATTCTCAGGGCGCCAGTTCTCTAACTCTC
GCTCTGAGATGAATGTGAGCACCTTGGAGCTGGGGGACTCGGCCCTTTATCTTTG
CGCCAGCAGCCACCATCAGGGGCCGTTAGAGACCCAGTACTTCGGGCCAGGCAC
GCGGCTCCTGGTGCTCGAG
SEQ ID NO: 51
P2A
RAKRSGSGATNFSLLKQAGDVEENPGP

What is claimed is:

1. A composition comprising one or a plurality of polypeptides comprising alpha variable regions or one or a plurality of beta variable regions of T-cell receptors (TCRs) specific for NY-ESO-1 (NY-ESO-1 TCR), CT83 (CT83-TCR), HCMV pp65 (HCMV pp65 TCR) or for HCMV IE-1 (HCMV IE-1 TCR), or any combination thereof.

2. The composition of claim 1, wherein the composition comprises: (a) at least one polypeptide comprising the alpha chain or region of a T-cell receptor specific for NY-ESO-1 (NY-ESO-1 TCR) and at least one polypeptide comprising the beta chain of a T-cell receptor specific for NY-ESO-1 (NY-ESO-1 TCR); (b) at least one polypeptide comprising the alpha chain or region of a T-cell receptor specific for CT83 (CT83-TCR) and at least one polypeptide comprising the beta chain of a T-cell receptor specific for CT83 (CT83-TCR); (c) at least one polypeptide comprising the alpha chain or region of a T-cell receptor specific for HCMV pp65 (HCMV pp65 TCR) and at least one polypeptide comprising the beta chain of a T-cell receptor specific for pp65 (pp65 TCR); or (d) at least one polypeptide comprising the alpha chain or region of a T-cell receptor specific for HCMV IE-1 (HCMV IE-1-TCR) and at least one polypeptide comprising the beta chain of a T-cell receptor specific for HCMV IE-1 (HCMV IE-1-TCR).

3. The composition of claim 1, wherein the composition comprises the alpha variable region of a HLA-A2 restricted HCMV pp65 TCR comprising a polypeptide comprising the amino acid sequence
(SEQ ID NO: 27)
METLLGLLILWLQLQWVSSKQEVTQIPAALSVPEGENLVLNCSFTDSAI
YNLQWFRQDPGKGLTSLLLIQSSQREQTSGRLNASLDKSSGRSTLYIAA
SQPGDSATYLCAVRPQGSTLGRLYFGRGTQLTVWPD
or
(SEQ ID NO: 29)
MEKNPLAAPLLILWFHLDCVSSILNVEQSPQSLHVQEGDSTNFTCSFPS
SNFYALHWYRWETAKSPEALFVMTLNGDEKKKGRISATLNTKEGYSYLY
IKGSQPEDSATYLCARNTGNQFYFGTGTSLTVIPN.
fragments or variants thereof which bind to the antigen with the same specificity as the reference (full length and unmodified) receptor, or a polypeptide comprising an amino acid sequence having 85%, 90%, or 95% homology to the polypeptide having the amino acid sequence of SEQ ID NO:27 or SEQ ID NO: 29, or having one or two conservative amino acid substitutions to the amino acid sequence of SEQ ID NO:27 or SEQ ID NO: 29, or having a sequence with one or two conservative amino acid substitutions to the amino acid sequence having 95% homology to the sequence of SEQ ID NO:27 or SEQ ID NO:29; and
optionally further wherein the composition comprises the beta variable region of a HLA-A2 restricted HCMV pp65 TCR comprising a polypeptide comprising the amino acid sequence MLSPDLPDSAWNTRLLCRVMLCLLGAGSVAAGVIQSPRHLIKEKRETATLKCYPIP RHDTVYWYQQGPGQDPQFLISFYEKMQSDKGSIPDRFSAQQFSGYHSELNMSSLEL GDSALYFCASSLENNQPQHFGDGTRLSILE (SEQ ID NO:28) or MSIGLLCCAALSLLWAGPVNAGVTQTPKFQVLKTGQSMTLQCAQDMNHEYMSW YRQDPGMGLRLIHYSVGAGITDQGEVPNGYNVSRSTTEDFPLRLLSAAPSQTSVYF CASSPITGTGDYGYTFGSGTRLTVVE (SEQ ID NO: 30), fragments or variants thereof which bind to the antigen with the same specificity as the reference (full length and unmodified) receptor, a polypeptide having 85%, 90% or 95% homology to a polypeptide comprising the sequence of SEQ ID NO:28 or SEQ ID NO: 30, or having a sequence with one or two conservative amino acid substitutions to SEQ ID NO:28 or SEQ ID NO:30, or one or two conservative amino acid substitutions to that of the amino acid sequence having 95% homology to that of the sequence of SEQ ID NO:28 or SEQ ID NO:.30, wherein the TCR further optionally comprises SEQ ID NO:27 and SEQ ID NO:28, or further optionally comprises SEQ ID NO:29 and SEQ ID NO:30.

4. The composition of claim 1, wherein the composition comprises alpha and beta variable regions of TCRs specific for NY-ESO-1 or CT83, wherein if the TCR is specific for NY-ESO-1 the alpha variable region optionally comprises a DP4-ESO-1 TCR polypeptide comprising the amino acid sequence METVLQVLLGILGFQAAWVSSQELEQSPQSLIVQEGKNLTINCTSSKTLYGLYWYK QKYGEGLIFLMMLQKGGEEKSHEKITAKLDEKKQQSSLHITASQPSHAGIYLCGAD IVDYGQNFVFGPGTRLSVLPY (SEQ ID NO: 3), fragments or variants thereof which bind to the antigen with the same specificity as the reference (full length and unmodified) receptor, a polypeptide comprising an amino acid sequence having 85%, 90%, or 95% homology to the amino acid sequence of SEQ ID NO: 3, a polypeptide having one or two conservative amino acid substitutions to the amino acid sequence of SEQ ID NO: 3, or a polypeptide having one or two conservative amino acid substitutions to the amino acid sequence having 95% homology to the sequence of SEQ ID NO:3 and the beta variable region of DP4-ESO-1 TCR optionally comprises a polypeptide comprising the amino acid sequence MLCSLLALLLGTFFGVRSQTIHQWPATLVQPVGSPLSLECTVEGTSNPNLYWYRQA AGRGLQLLFYSVGIGQISSEVPQNLSASRPQDRQFILSSKKLLLSDSGFYLCAWRRR GYEQYFGPGTRLTVTE (SEQ ID NO: 4), fragments or variants thereof which bind to the antigen with the same specificity as the reference (full length and unmodified) receptor, a polypeptide comprising an amino acid sequence having 85%, 90%, or 95% homology to the amino acid sequence of SEQ ID NO: 4, a polypeptide having one or two conservative amino acid substitutions to the amino acid sequence of SEQ ID NO: 4, or a polypeptide having one or two conservative amino acid substitutions to the amino acid sequence having 95% homology to the sequence of SEQ ID NO:4; and
wherein if the TCR is specific for CT83 the alpha variable region optionally comprises an A2-CT83 TCR a polypeptide comprising the amino acid sequence MKTFAGFSFLFLWLQLDCMSRGEDVEQSLFLSVREGDSSVINCTYTDSSSTYLYWY KQEPGAGLQLLTYIFSNMDMKQDQRLTVLLNKKDKHLSLRIADTQTGDSAIYFCA EKSGYSGAGSYQLTFGKGTKLSVIPN (SEQ ID NO: 5), fragments or variants thereof which bind to the antigen with the same specificity as the reference (full length and unmodified) receptor, a polypeptide comprising an amino acid sequence having 85%, 90%, or 95% homology to the amino acid sequence of SEQ ID NO: 5, a polypeptide having one or two conservative amino acid substitutions to the amino acid sequence of SEQ ID NO: 5, or a polypeptide having one or two conservative amino acid substitutions to the amino acid sequence having 95% homology to the sequence of SEQ ID NO:5 and the beta variable region of the CT83 TCR optionally comprises an A2-CT83 TCR polypeptide comprising the amino acid sequence MLSLLLLLLGLGSVFSAVISQKPSRDICQRGTSLTIQCQVDSQVTMMFWYRQQPGQ SLTLIATANQGSEATYESGFVIDKFPISRPNLTFSTLTVSNMSPEDSSIYLCSVQDSEA FFGQGTRLTVVE (SEQ ID NO: 6), fragments or variants thereof which bind to the antigen with the same specificity as the reference (full length and unmodified) receptor, a polypeptide comprising an amino acid sequence having 85%, 90%, or 95% homology to the amino acid sequence of SEQ ID NO: 6, a polypeptide having one or two conservative amino acid substitutions to the amino acid sequence of SEQ ID NO: 6, or a polypeptide having one or two conservative amino acid substitutions to the amino acid sequence having 95% homology to the sequence of SEQ ID NO:6.

5. The composition of claim 1, wherein the composition comprises the alpha variable region of a HLA-A2 restricted HCMV IE-1 TCR, wherein the alpha variable region of a HLA-A2 restricted HCMV IE-1 TCR is a polypeptide comprising the amino acid sequence MLLITSMLVLWMQLSQVNGQQVMQIPQYQHVQEGEDFTTYCNSSTTLSNIQWYK QRPGGHPVFLIQLVKSGEVKKQKRLTFQFGEAKKNSSLHITATQTTDVGTYFCAGH IYGGSQGNLIFGKGTKLSVKPN (SEQ ID NO: 32), a polypeptide comprising an amino acid sequence having 85%, 90%, or 95% homology to that of the sequence of SEQ ID NO: 32, a polypeptide having one or two conservative amino acid substitutions to SEQ ID NO:32, or a polypeptide having one or two conservative amino acid substitutions to that of the amino acid sequence having 95% homology to that of the sequence of SEQ ID NO:32, and optionally wherein the composition further comprises the beta variable region of a HLA-A2-restricted IE-1 TCR, wherein the beta variable region of the HLA-A2-restricted IE-1 TCR comprises a polypeptide comprising the amino acid sequence MGSRLLCWVLLCLLGAGPVKAGVTQTPRYLIKTRGQQVTLSCSPISGHRSVSWYQ QTPGQGLQFLFEYFSETQRNKGNFPGRFSGRQFSNSRSEMNVSTLELGDSALYLCA SSHHQGPLETQYFGPGTRLLVLE (SEQ ID NO: 33), a polypeptide comprising an amino acid sequence having 85%, 90%, or 95% homology to that of the sequence of SEQ ID NO:336, a polypeptide having one or two conservative amino acid substitutions to SEQ ID NO:33, or a polypeptide having one or two conservative amino acid substitutions to that of the amino acid sequence having 95% homology to that of the sequence of SEQ ID NO:33.

6. A. chimeric TCR polypeptide comprising a cancer antigen specific TCR variable region fused to a constant region selected from a modified human TCR alpha or beta constant region and a non-human TCR alpha or beta constant region, optionally a murine TCR alopha or beta constant region, wherein the alpha and beta variable regions of the TCR variable regions fused to modified or non-human alpha or beta constant chain regions comprise:
a. any combination of alpha and beta TCR variable regions recited in any of claim 3, or 5.; or
b. alpha and beta variable regions of TCRs specific for a cancer antigen selected from NY-ESO-1 and CT83, wherein if the TCR is specific for NY-ESO-1 the alpha variable region optionally comprises a DP4-ESO-1 TCR polypeptide comprising the amino acid sequence METVLQVLLGILGFQAAWVSSQELEQSPQSLIVQEGKNLTINCTSSKTL YGLYWYKQKYGEGLIFLMMLQKGGEEKSHEKITAKLDEKKQQSSLHIT ASQPSHAGIYLCGADIVDYGQNFVFGPGTRLSVLPY (SEQ ID NO: 3), fragments or variants thereof which bind to the antigen with the same specificity as the reference (full length and unmodified) receptor, a polypeptide comprising an amino acid sequence having 85%, 90%, or 95% homology to the amino acid sequence of SEQ ID NO: 3, a polypeptide having one or two conservative amino acid substitutions to the amino acid sequence of SEQ ID NO: 3, or a polypeptide having one or two conservative amino acid substitutions to the amino acid sequence having 95% homology to the sequence of SEQ ID NO:3 and the beta variable region of DP4-ESO-1 TCR optionally comprises a polypeptide comprising the amino acid sequence MLCSLLALLLGTFFGVRSQTIHQWPATLVQPVGSPLSLECTVEGTSNPN LYWYRQAAGRGLQLLFYSVGIGQISSEVPQNLSASRPQDRQFILSSKKL LLSDSGFYLCAWRRRGYEQYFGPGTRLTVTE (SEQ ID NO: 4), fragments or variants thereof which bind to the antigen with the same specificity as the reference (full length and unmodified) receptor, a polypeptide comprising an amino acid sequence having 85%, 90%, or 95% homology to the amino acid sequence of SEQ ID NO: 4, a polypeptide having one or two conservative amino acid substitutions to the amino acid sequence of SEQ ID NO: 4, or a polypeptide having one or two conservative amino acid substitutions to the amino acid sequence having 95% homology to the sequence of SEQ ID NO:4; and
c. wherein if the TCR is specific for CT83 the alpha variable region optionally comprises an A2-CT83 TCR a polypeptide comprising the amino acid sequence MKTFAGFSFLFLWLQLDCMSRGEDVEQSLFLSVREGDSSVINCTYTDSS STYLYWYKQEPGAGLQLLTYIFSNMDMKQDQRLTVLLNKKDKHLSLR IADTQTGDSAIYFCAEKSGYSGAGSYQLTFGKGTKLSVIPN (SEQ ID NO: 5), fragments or variants thereof which bind to the antigen with the same specificity as the reference (full length and unmodified) receptor, a polypeptide comprising an amino acid sequence having 85%, 90%, or 95% homology to the amino acid sequence of SEQ ID NO: 5, a polypeptide having one or two conservative amino acid substitutions to the amino acid sequence of SEQ ID NO: 5, or a polypeptide having one or two conservative amino acid substitutions to the amino acid sequence having 95% homology to the sequence of SEQ ID NO:5 and the beta variable region of the CT83 TCR optionally comprises an A2-CT83 TCR polypeptide comprising the amino acid sequence MLSLLLLLLGLGSVFSAVISQKPSRDICQRGTSLTIQCQVDSQVTMMFW YRQQPGQSLTLIATANQGSEATYESGFVIDKFPISRPNLTFSTLTVSNMS PEDSSIYLCSVQDSEAFFGQGTRLTVVE (SEQ ID NO: 6), fragments or variants thereof which bind to the antigen with the same specificity as the reference (full length and unmodified) receptor, a polypeptide comprising an amino acid sequence having 85%, 90%, or 95% homology to the amino acid sequence of SEQ ID NO: 6, a polypeptide having one or two conservative amino acid substitutions to the amino acid sequence of SEQ ID NO: 6, or a polypeptide having one or two conservative amino acid substitutions to the amino acid sequence having 95% homology to the sequence of SEQ ID NO:6.

7. The chimeric TCR receptor of claim 6, wherein the alpha chain constant region is selected from: a modified human TCR alpha constrant chain region (TRAC) comprising the sequence IQNPDPAVYQLRDSKSSDKSVCLFTDFDSQTNVSQSKDSDVYITDKTVLDMRSMD FKSNSAVAWSNKSDFACANAFNNSIIPEDTFFPSPESSCDVKLVEKSFETDTNLNFQ NLSVIGFRILLLKVAGFNLLMTLRLWSS) (Seq ID NO: 10), and a murine alpha chain constant region (trac) comprising the sequence IQNPEPAVYQLKDPRSQDSTLCLFTDFDSQINVPKTMESGTFITDKTVLDMKAMDS KSNGAIAWSNQTSFTCQDIFKETNATYPSSDVPCDATLTEKSFETDMNLNFQNLSV MGLRILLLKVAGFNLLMTLRLWSS (SEQ ID NO:13); and
wherein the beta chain constant region is selected from: a modified human TCR beta constrant region type 2 having the sequence DLKNVFPPEVAVFEPSEAEISHTQKATLVCLATGFYPDHVELSWWVNGKEVHSGV STDPQPLKEQPALNDSRYCLSSRLRVSATFWQNPRNHFRCQVQFYGLSENDEWTQ DRAKPVTQIVSAEAWGRADCGFTSESYQQGVLSATILYEILLGKATLYAVLVSAL VLMAMVKRKDSRG) (Seq ID NO: 12), a modified human TCR beta constant region type 1 (TRBC1) having the sequence DLNKVFPPEVAVFEPSEAEISHTQKATLVCLATGFFPDHVELSWWVNGKEVHSGV STDPQPLKEQPALNDSRYCLSSRLRVSATFWQNPRNHFRCQVQFYGLSENDEWTQ DRAKPVTQIVSAEAWGRADCGFTSVSYQQGVLSATILYEILLGKATLYAVLVSAL VLMAMVKRKDF) (SEQ ID NO:11), a murine beta chain constant region type 1 (trbc1) having the sequence DLRNVTPPKVSLFEPSKAEIANKQKATLVCLARGFFPDHVELSWWVNGKEVHSG VSTDPQAYKESNYSYCLSSRLRVSATFWHNPRNHFRCQVQFHGLSEEDKWPEGSP KPVTQNISAEAWGRADCGITSASYQQGVLSATILYEILLGKATLYAVLVSTLVVM AMVKRKNS (SEQ ID NO:14), and a murine beta chain constant region type 2 (trbc2) having the sequence
(SEQ ID NO: 15)
DLRNVTPPKVSLFEPSKAEIANKQKATLVCLARGFFPDHVELSWWVNGK
EVHSGVSTDPQAYKESNYSYCLSSRLRVSATFWHNPRNHFRCQVQFHGL
SEEDKWPEGSPKPVTQNISAEAWGRADCGITSASYHQGVLSATILYEIL
LGKATLYAVLVSGLVLMAMVKKKNS.

8. The chimeric TCR receptor of claim 7, wherein the TCRs are specific for a cancer antigen selected from NY-ESO-1, CT83 (“CT83-TCR”), HCMV PP65 and HCMV IE1.

9. The chimeric TCR receptor of claim 7, wherein the chimeric TCR receptor is specific for CT83 and comprises a chimeric alpha chain comprising a HLA-A2 restricted CT83 TCR alpha chain variable region fused with a murine alpha constant domain, comprising a polypeptide selected from a polypeptide having SEQ ID NO:20, a polypeptide comprising an amino acid sequence having 95% sequence identity to the amino acid sequence of SEQ ID NO: 20, a polypeptide having one or two conservative amino acid substitutions to the amino acid sequence of SEQ ID NO: 20, and a polypeptide having one or two conservative amino acid substitutions to the amino acid sequence having 95% sequence identity to the sequence of SEQ ID NO:20, and wherein the chimeric TCR receptor specific for CT83 further comprises a chimeric beta chain comprising a HLA-A2-restricted CT83 TCR beta chain variable domain fused with murine Beta constant domain 2 selected from a polypeptide having SEQ ID NO:21, a polypeptide having 95% sequence identity to SEQ ID NO:21, a polypeptide having one or two conservative amino acid substitutions to the amino acid sequence of to SEQ ID NO:21, and a polypeptide having one or two conservative amino acid substitutions to the amino acid sequence having 95% sequence identity to the sequence of SEQ ID NO:21.

10. The chimeric TCR receptor of claim 7, wherein the chimeric TCR receptor is specific for NY-ESO-1 and comprises a chimeric alpha chain selected from:
a HLA-A2-restricted NY-ESO-1 TCR (S2) alpha chain variable domain fused with murine alpha constant domain having SEQ ID NO: 22,
a polypeptide having 95% sequence identity to SEQ ID NO:22,
a polypeptide having one or two conservative amino acid substitutions to the amino acid sequence of to SEQ ID NO:22,
a polypeptide having one or two conservative amino acid substitutions to the amino acid sequence having 95% sequence identity to the sequence of SEQ ID NO:22;
a HLA-A2-restricted NY-ESO-1 TCR (S5) alpha chain variable domain fused with a murine alpha constant domain having SEQ ID NO:24,
a polypeptide having 95% sequence identity to SEQ ID NO:24,
a polypeptide having one or two conservative amino acid substitutions to the amino acid sequence of to SEQ ID NO:24, and a polypeptide having one or two conservative amino acid substitutions to the amino acid sequence having 95% sequence identity to the sequence of SEQ ID NO:24; and
wherein the chimeric TCR receptor specific for NY-ESO-1 further comprises a chimeric beta chain selected from:
a HLA-A2-restricted NY-ESO-1 TCR (S2) (G50A, A51E) beta chain variable domain fused with murine beta constant domain 2 comprising SEQ ID NO:23,
a polypeptide having 95% sequence identity to SEQ ID NO:23,
a polypeptide having one or two conservative amino acid substitutions to the amino acid sequence of to SEQ ID NO:23,
a polypeptide having one or two conservative amino acid substitutions to the amino acid sequence having 95% sequence identity to the sequence of SEQ ID NO:23;
a HLA-A2-restricted NY-ESO-1 TCR (S5) (G50A, A51E, A97L) Beta chain variable domain fused with murine Beta constant domain 2 having SEQ ID NO:25
a polypeptide having 95% sequence identity to SEQ ID NO:25,
a polypeptide having one or two conservative amino acid substitutions to the amino acid sequence of to SEQ ID NO:25, and
a polypeptide having one or two conservative amino acid substitutions to the amino acid sequence having 95% sequence identity to the sequence of SEQ ID NO:25.

11. The chimeric TCR receptor of any one of claim 6-9 or 10, wherein the TCR is fused to a signaling component optionally selected from ZAP 300 (SEQ ID NO: 16) or ZAP327 (SEQ ID NO: 17).

12. A nucleic acid, vector, or cell comprising a nucleic or vector encoding any of the sequences of the polypeptides in the compositions or chimeric TCRs of any one of claim 6, 9, or 10, optionally further encoding a signaling component wherein the signaling component is optionally selected from ZAP 300 (SEQ ID NO: 16) or ZAP327 (SEQ ID NO: 17).

13. A composition comprising a therapeutically effective amount of one or more TCR T-cells, wherein the TCR T cell has been engineered to express any of the TCR receptors of claim 6, 9 or 10.

14. An isolated nucleic acid encoding a shRNA for knocking down a gene for the enhancement of antitumor activity of TCR-transduced T cells i6 vivo, wherein the nucleic acid sequences of shRNA target immune system negative signaling molecules which are optionally selected from checkpoint proteins and/or immune suppressor proteins.

15. The isolated nucleic acid of claim 14, wherein the shRNA targets are selected from programmed cell death protein (PD1), (SEQ ID NO: 7), von Hippel-Lindau tumor suppressor (VHL) (SEQ ID NO: 8), and/or protein phosphatase 2 regulatory subunit B delta (PPP2R2D) (SEQ ID NO: 9).

16. A method of stimulating an immunological response against a cancer or treating, inhibiting, and/or preventing a cancer, the method comprising administering to a subject a composition comprising a therapeutically effective amount of a composition comprising a TCR alpha or beta variable region polypeptide of any of claim 6, 9, or 10.

17. A composition comprising chimeric antigen receptors or T cells expressing a CAR, wherein the CAR comprises an antigen recognition moiety, a transmembrane domain, and an intracellular T-cell activation moiety, wherein the intracellular T-cell activation moieties is optionally selected from CD28 or 4-1BB costimulatory signaling domain fused with a signaling domain, further wherein the signaling domain is optionally selected from ZAP300 (SEQ ID NO: 16) or ZAP327 (SEQ ID NO: 17), and wherein the antigen recognition moiety is optionally a single chain variable fragment (ScFv).

18. A method of treating cancer in a subject having or suspected of having cancer by administering to the subject a composition comprising the TCR-T T cells or CAR-T T cells of claim 13 or 17.

19. A method of prolonging T cell persistence or reducing T cell exhaustion in a subject by modulating TCR-T cell signaling and function by administering to a subject a composition comprising the TCR-T cells or CAR-T cells of claim 13 or 17.

20. The method of claim 19, wherein TCR or CAR signaling domains are regulated, or knocked down by negative signaling molecules which are selected from: PD-1, VHL, PPP2R2D and epigenetic factors which can include or exclude JMJD3 and LSD1.

21. The method of claim 20, wherein TCR or CAR signaling domains are regulated, or knocked down by negative signaling molecules which are selected from: PD-1, VHL, PPP2R2D and epigenetic factors which can include or exclude JMJD3 and LSD1.

22. A method for prolonging TCR-T and CAR-T cell persistence by direct manipulation of TCR or CAR signaling domains or by knockdown/knockout of negative signaling molecules, wherein the negative signaling molecules are selected from: indoleamine (2,3)-dioxygenase (IDO) (including isoforms IDO1 and IDO2), OX40, CTLA-4 (programmed cytotoxic T-lymphocyte antigen 4), PD-1 (programmed death 1), PD-L1 (programmed death ligand 1), PD-L2, lymphocyte activation gene 3 (LAG3), and B7 homolog 3 (B7-H3).

23. The method of claim 22, wherein the negative signaling molecules are PD-1, VHL, PPP2R2D, and epigenetic factors which can include or exclude JMJD3 and LSD1.

24. The method of claim 23, further comprising the step of forcing expression of chemokine receptors, whereby T cell trafficking into tumor cells is enhanced, wherein forcing the expression of chemokine receptors comprises fusing the CAR or TCR with a chemokine receptor, wherein the chemokine receptor is optionally selected from CCR5, CCR2, and CXCR3.


SEQUENCE LISTING (52 sequences)

SEQ ID NO: 1 - protein, 14 aa, Homo sapiens
SLLMWITQCFLPVF

SEQ ID NO: 2 - protein, 9 aa, Homo sapiens
KLVELEHTL

SEQ ID NO: 3 - protein, 133 aa, Homo sapiens
METVLQVLLGILGFQAAWVSSQELEQSPQSLIVQEGKNLTINCTSSKTLYGLYWYKQKYGEGLIFLMMLQKGGEEKSHEKITAKLDEKKQQSSLHITASQPSHAGIYLCGADIVDYGQNFVFGPGTRLSVLPY

SEQ ID NO: 4 - protein, 129 aa, Homo sapiens
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWRRRGYEQYFGPGTRLTVTE

SEQ ID NO: 5 - protein, 137 aa, Homo sapiens
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEKSGYSGAGSYQLTFGKGTKLSVIPN

SEQ ID NO: 6 - protein, 127 aa, Homo sapiens
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVQDSEAFFGQGTRLTVVE

SEQ ID NO: 7 - DNA, 21 bp, Artificial Sequence
  Description of Artificial Sequence Synthetic oligonucleotide
ccgtgtcacacaactgcccaa

SEQ ID NO: 8 - DNA, 21 bp, Artificial Sequence
  Description of Artificial Sequence Synthetic oligonucleotide
caggagcgcattgcacatcaa

SEQ ID NO: 9 - DNA, 21 bp, Artificial Sequence
  Description of Artificial Sequence Synthetic oligonucleotide
aaggtcattactcagaataaa

SEQ ID NO: 10 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 11 - protein, 176 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 12 - protein, 178 aa, Homo sapiens
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 13 - protein, 136 aa, Mus musculus
IQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 14 - protein, 172 aa, Mus musculus
DLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 15 - protein, 172 aa, Mus musculus
DLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 16 - protein, 320 aa, Homo sapiens
TSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 17 - protein, 293 aa, Homo sapiens
DKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 18 - DNA, 5374 bp, Homo sapiens
aaaaatccctccccggcggcggcggcggcggcggcggcgccggcggtggtggcggccccggggctgagcgctcggctgcagcggcgcggaggccgtctccctggtctgccgcggtccccgcccgtcccgccgccggctgccatggcaggagccggaggcggcggctgccccgcgggcggcaacgacttccagtggtgcttctcgcaggtcaagggggccatcgacgaggacgtggccgaagcggacatcatttccaccgttgagtttaattactctggagatcttcttgcaacaggagacaagggcggcagagttgttatttttcagcgtgaacaagagaataaaagccgccctcattctaggggagaatataatgtttacagcacctttcaaagtcatgaaccggagtttgactatttgaaaagtctagaaattgaggaaaaaattaataaaattaggtggttaccacaacagaatgctgctcattttctactgtctacaaatgataaaactataaaattatggaaaataagtgaacgggataaaagagcagaaggttataacctgaaagacgaagatggaagacttcgagacccatttaggatcacggcgctacgggtcccaatattgaagcccatggatcttatggtagaagcgagtccacggcgaatttttgcaaatgctcacacatatcatataaattccatttcagtaaatagtgatcatgaaacatatctttctgcagatgacctgagaattaatttatggcacttagaaatcacagatagaagctttaacatcgtggacatcaagcctgctaacatggaggagctgaccgaagtcatcactgcagccgagttccacccgcaccagtgcaacgtgttcgtctacagcagtagcaaagggaccatccgcctgtgtgacatgcgctcctcggccctgtgcgacagacactccaagttttttgaagagcctgaagatcccagcagtaggtccttcttctcagaaataatttcatccatatccgatgtaaaattcagtcatagtgggcggtacatgatgaccagagactacctgtcggtgaaggtgtgggacctcaacatggagagcaggccggtggagacccaccaggtccacgagtacctgcgcagcaagctctgctctctctatgagaacgactgcatctttgacaagtttgagtgttgctggaacggttcggatagcgccatcatgaccgggtcctataacaacttcttcaggatgtttgatagagacacgcggagggatgtgaccctggaggcctcgagagagagcagcaaaccgcgcgccagcctcaaaccccggaaggtgtgtacggggggtaagcggaggaaagacgagatcagtgtggacagtctggacttcaacaagaagatcctgcacacagcctggcaccccgtggacaatgtcattgccgtggctgccaccaataacttgtacatattccaggacaaaatcaactagagacgcgaacgtgaggaccaagtcttgtcttgcatagttaagccggacatttttctgtcagagaaaaggcatcattgtccgctccattaagaacagtgacgcacctgctacttcccttcacagacacaggagaaagccgcctccgctggaggcccggtgtggttccgcctcggcgaggcgcgagacaggcgctgctgctcacgtggagacgctctcgaagcagagttgacggacactgctcccaaaaggtcattactcagaataaatgtatttatttcagtccgagccttcctttccaatttatagaccaaaaaattaacatccaagagaaaagttattgtcagataccgctctttctccaactttccctctttctctgccatcacacttgggccttcactgcagcgtggtgtggccaccgtccgtgtcctctcggccttcctccgagtccaggtggactctgtggatgtgtggatgtggcccgagcaggctcaggcggccccactcacccacagcatccgccgccaccccttcgggtgtgagcgctcaataaaaacaacacactataaagtgtttttaaatccaaacagaagtattgtctttttatttaattttattgcatagaatgaagttatgcagggttcttctttggaactaactgtttgagaaatgtgtgtccttctttggcagcgtgggggtatgtgtgcagcatttctgtccccaagctgctgcccgctgtgtttctgtagaagtagcccatcagatacaccaggtaaggtctgggaaaagccagaaccgaggccacctctgagaaagaaaattgcaaggaagaaccaaaattgctgccatccagtaagcctggtttaaagtagcttcaaattcactacagttgaagaaatacgtgcttgctttctaaggcttttgaaaaagcactttggggaaagtatactttttaaacattgctaaaattacatgcatgctaaatttcatcctgcatttcaagccagcaaaattaagcaattttaattacacgatgccaccagtacgttggtttattttcaaagtaggatctttgataccagaaatcaagattttccaagacaaataatacagagctgtaccaatgctgagtgaccagagcttgcttccgtgggatttaacacacgcccactacgtgtccccgaggggagtggggagtcgggctcccgtgcccctgtggagatggaggtgtgtgctgatcccccgtccgcctgtggagatgaaggtgtgtgttgattcctcctgctgctgtggagatggaggtgtgtgctgatcgcccgtcaccctgtctagatgaaggtatgtgctgattgcctgtgcccctgtggagaaggaggtgtgtgctgatcccctgtgccctgtggagatggaggtgtgtgctgctccctcgtgcccctgtggagatggaggtgtgtgctgatcccccatcccatccccctgtctagatgaaggtgtgtgctgattcctcatgcccctgtctggatgaaggtgtgtgctgatcccccgtccccctgtggagatgaaggtgtgtgctgatcccccgtgcccctgtggagatgaaggtgcgtgctgatcccccatctccctgtggagatgaaggtgtgtgctgatccccatccccctgtggagatgaagggatatgctgatcccctgtcccactgtggagatgaaggggtgtgctgatcccccgtccccctgtggagatgaaggtgtgtgctgatcccccatctccctgtggagatgaaggggtgtgccaatcccccgcgcccctgtggagatggaggcgtgtgctgatccccatccccctgtggagatgaaggtgtgtgctgatcccccatccccatgtggagatgaaggggtgtgttgatcccctgtccccctgtggagatggtgtgtgctgatccccgtccccctgtggagatgaaggtgtgtgctgatcccccatctccctgtgaagatgaaggggtgtgctgatcccccgtgcccctgtggacatggaggcgtgtgctgatcccccgtccccctgtggagatgaaggtgtgtgctgatcccccatccccatgtggagatgaaggggtgtgttgatcccccgtccccctgtggagatgaaggtgtgtgctgatccccgtccccctgtggagatgaaggggtgtgctgatcccccatgccctgtggagatggaggtgtgtgctgatccctcgtgcccctgtggagatagaggtgtgtactgatcccctgttcccctatctagatgaaggtgtgtactgctgatcccccgtccccctgtggagatgaaggtgtgtgctgatcccccatccccctgtggagatgaaggggtgtgctgatcccccgtcttcctgtggagatgaaggtgtgtgctgattcctcatgcccctgtggagatggaggtgtgtgctgatcccccatccccctgtggggatgaaggtgtgtgctgatcccccatccccctgtggagatgaaggggtatgctgatcccccgtccccctgtggagatgaaggtgtgtgctgatcccccatccccctgtggagatgaaggggtatgctgatcccctgtccccctgtggggatgaaggtgtgtgctgatctcttgtccccctgtggagatggaggtgtgtgctgatcccccatccccctgtggagatgaagatgtgtgctgatcccccgtcctcctgtggagatgaaggtgtgtgctgcttcctcgtgcccctgtggagatggaggtgtgtgctggtcccccgtccccctgtggagatggaggtgtgtgctgatcccccgtccccctgtggagatggaggcgtgtgctgatcccccatccccctgtggagatgaaggcgtgtgctgatccgccatcccactgtggagatgaaggcgtgtgctgatcccccatccccctgtggagataaaggtgtgtgctgatcccccatccccctgtggagatgaaggcgtgtgctgatccgccatcccactgtggagatgaaggcgtgtgctgatcccccatccccctgtggagatgaaggcgtgtgctcatcccccatccccctgtggagatgaaggcgtgtgctgatccgccatcccactgtggagatgaaggtgtgtgctgatcccccatccccctgtggagatgaaggtgtgtgctgatccccggtccccctgtggagatgaaggtgtgtgctgatcccccgtcctcctgtggagatgaaggcgtgtgctgatcccccgtccccctgtggagatgaaggcgtgtgctgatccgccgtccccctgtggagatgaaggcgtgtgctgatccgccatccccctgtggagatgaaggcgtgtgctcatcccccatccccctgtggagatgaaggcgtgtgctgatccgccatcccactgtggagatgaaggtgtgtgctgatcccccatccccctgtggagatgaaggcgtgtgctgatccccggtccccctgtggagatgaaggtgtgtgttgatcccccatccccctgtggaggtgaaggtgtgtgctgattactaatccacatgggcaagaacaggatgtccgtcattaccctgaatcaatgctaacacagtgccacttgagttcacattaagttagaaattgagaatctaaaggtacctttattttaactaaaaaataatttatatactgtatattgattgtgacacaatttacaaagtctgaggtgtggaacagttatttaagcattagtcaaccctggtccttaagacagttctagtaaaatgggattgtatatatttgttcaactattttgaccaaaaagttcaataaattttaaatgtttaactgga

SEQ ID NO: 19 - DNA, 6411 bp, Homo sapiens
aaaaatccctccccggcggcggcggcggcggcggcggcgccggcggtggtggcggccccggggctgagcgctcggctgcagcggcgcggaggccgtctccctggtctgccgcggtccccgcccgtcccgccgccggctgccatggcaggagccggaggcggcggctgccccgcgggcggcaacgacttccagtggtgcttctcgcaggtcaagggggccatcgacgaggacgtggccgaagggcgtggagaggagagctcggggctgctgccacagatcatgctgagccgccagcagctccacctgcccttggctttgctcatccgtggaaatgacaacagcgtgaaaacggcagatgatgtacaaggattatgatgaaaatagtcttgacctcacggaccctctgagaggttcttgaccgttgcaccctcaagggcccacagaccgcactttgagaactgctgcttgaatgcaagctattagcagtaggagtgtcaaggacagggcagacttcaggattaactgttccttctcatctttaccagattgtactggttttgtctcaaaatacaaaaatagagtgtttattatgtaaagaaaatcaaatgatagaacagctgagaacagctggatcaagagcacagagcggaagttcctcttcatcccactgctgtcctggagtcttcctcaggacccactgctcctaactgtatctttgtagatcattttctatgtgtttatttatttatattttaaaacctagtttgtgtataagcaatcttttttaatccacaaatgtggaatcttattctagactttgaataagattttctgtattagtcagtattgttttttgctacttcttttcctttcctttccttttttggaaatagggtcttactcctctgtcacccaggctggagtgcagtggtgcgatcagagctcactgcagcctgatcaggctcaagcaatcctctccctgctcagcctcctgagtagctggaactataggcttgcactgccatgcccagcttttttctattttttgtagagacagggttttgctgtgttgcccaggatggtcttgaactcctggactcaagtgatcctcccacctcggcctcccaaaatgctgggaatacaagcatgagccatggcatgtggccgccacttgccttttttcattaagagtctataattggagatcttacagggaactgggctttctgctcagccatactcgagctcatttgacccccggccactgccccttgaaatcggacatcatttccaccgttgagtttaattactctggagatcttcttgcaacaggagacaagggcggcagagttgttatttttcagcgtgaacaagagaataaaagccgccctcattctaggggagaatataatgtttacagcacctttcaaagtcatgaaccggagtttgactatttgaaaagtctagaaattgaggaaaaaattaataaaattaggtggttaccacaacagaatgctgctcattttctactgtctacaaatgataaaactataaaattatggaaaataagtgaacgggataaaagagcagaaggttataacctgaaagacgaagatggaagacttcgagacccatttaggatcacggcgctacgggtcccaatattgaagcccatggatcttatggtagaagcgagtccacggcgaatttttgcaaatgctcacacatatcatataaattccatttcagtaaatagtgatcatgaaacatatctttctgcagatgacctgagaattaatttatggcacttagaaatcacagatagaagctttaacatcgtggacatcaagcctgctaacatggaggagctgaccgaagtcatcactgcagccgagttccacccgcaccagtgcaacgtgttcgtctacagcagtagcaaagggaccatccgcctgtgtgacatgcgctcctcggccctgtgcgacagacactccaagttttttgaagagcctgaagatcccagcagtaggtccttcttctcagaaataatttcatccatatccgatgtaaaattcagtcatagtgggcggtacatgatgaccagagactacctgtcggtgaaggtgtgggacctcaacatggagagcaggccggtggagacccaccaggtccacgagtacctgcgcagcaagctctgctctctctatgagaacgactgcatctttgacaagtttgagtgttgctggaacggttcggatagcgccatcatgaccgggtcctataacaacttcttcaggatgtttgatagagacacgcggagggatgtgaccctggaggcctcgagagagagcagcaaaccgcgcgccagcctcaaaccccggaaggtgtgtacggggggtaagcggaggaaagacgagatcagtgtggacagtctggacttcaacaagaagatcctgcacacagcctggcaccccgtggacaatgtcattgccgtggctgccaccaataacttgtacatattccaggacaaaatcaactagagacgcgaacgtgaggaccaagtcttgtcttgcatagttaagccggacatttttctgtcagagaaaaggcatcattgtccgctccattaagaacagtgacgcacctgctacttcccttcacagacacaggagaaagccgcctccgctggaggcccggtgtggttccgcctcggcgaggcgcgagacaggcgctgctgctcacgtggagacgctctcgaagcagagttgacggacactgctcccaaaaggtcattactcagaataaatgtatttatttcagtccgagccttcctttccaatttatagaccaaaaaattaacatccaagagaaaagttattgtcagataccgctctttctccaactttccctctttctctgccatcacacttgggccttcactgcagcgtggtgtggccaccgtccgtgtcctctcggccttcctccgagtccaggtggactctgtggatgtgtggatgtggcccgagcaggctcaggcggccccactcacccacagcatccgccgccaccccttcgggtgtgagcgctcaataaaaacaacacactataaagtgtttttaaatccaaacagaagtattgtctttttatttaattttattgcatagaatgaagttatgcagggttcttctttggaactaactgtttgagaaatgtgtgtccttctttggcagcgtgggggtatgtgtgcagcatttctgtccccaagctgctgcccgctgtgtttctgtagaagtagcccatcagatacaccaggtaaggtctgggaaaagccagaaccgaggccacctctgagaaagaaaattgcaaggaagaaccaaaattgctgccatccagtaagcctggtttaaagtagcttcaaattcactacagttgaagaaatacgtgcttgctttctaaggcttttgaaaaagcactttggggaaagtatactttttaaacattgctaaaattacatgcatgctaaatttcatcctgcatttcaagccagcaaaattaagcaattttaattacacgatgccaccagtacgttggtttattttcaaagtaggatctttgataccagaaatcaagattttccaagacaaataatacagagctgtaccaatgctgagtgaccagagcttgcttccgtgggatttaacacacgcccactacgtgtccccgaggggagtggggagtcgggctcccgtgcccctgtggagatggaggtgtgtgctgatcccccgtccgcctgtggagatgaaggtgtgtgttgattcctcctgctgctgtggagatggaggtgtgtgctgatcgcccgtcaccctgtctagatgaaggtatgtgctgattgcctgtgcccctgtggagaaggaggtgtgtgctgatcccctgtgccctgtggagatggaggtgtgtgctgctccctcgtgcccctgtggagatggaggtgtgtgctgatcccccatcccatccccctgtctagatgaaggtgtgtgctgattcctcatgcccctgtctggatgaaggtgtgtgctgatcccccgtccccctgtggagatgaaggtgtgtgctgatcccccgtgcccctgtggagatgaaggtgcgtgctgatcccccatctccctgtggagatgaaggtgtgtgctgatccccatccccctgtggagatgaagggatatgctgatcccctgtcccactgtggagatgaaggggtgtgctgatcccccgtccccctgtggagatgaaggtgtgtgctgatcccccatctccctgtggagatgaaggggtgtgccaatcccccgcgcccctgtggagatggaggcgtgtgctgatccccatccccctgtggagatgaaggtgtgtgctgatcccccatccccatgtggagatgaaggggtgtgttgatcccctgtccccctgtggagatggtgtgtgctgatccccgtccccctgtggagatgaaggtgtgtgctgatcccccatctccctgtgaagatgaaggggtgtgctgatcccccgtgcccctgtggacatggaggcgtgtgctgatcccccgtccccctgtggagatgaaggtgtgtgctgatcccccatccccatgtggagatgaaggggtgtgttgatcccccgtccccctgtggagatgaaggtgtgtgctgatccccgtccccctgtggagatgaaggggtgtgctgatcccccatgccctgtggagatggaggtgtgtgctgatccctcgtgcccctgtggagatagaggtgtgtactgatcccctgttcccctatctagatgaaggtgtgtactgctgatcccccgtccccctgtggagatgaaggtgtgtgctgatcccccatccccctgtggagatgaaggggtgtgctgatcccccgtcttcctgtggagatgaaggtgtgtgctgattcctcatgcccctgtggagatggaggtgtgtgctgatcccccatccccctgtggggatgaaggtgtgtgctgatcccccatccccctgtggagatgaaggggtatgctgatcccccgtccccctgtggagatgaaggtgtgtgctgatcccccatccccctgtggagatgaaggggtatgctgatcccctgtccccctgtggggatgaaggtgtgtgctgatctcttgtccccctgtggagatggaggtgtgtgctgatcccccatccccctgtggagatgaagatgtgtgctgatcccccgtcctcctgtggagatgaaggtgtgtgctgcttcctcgtgcccctgtggagatggaggtgtgtgctggtcccccgtccccctgtggagatggaggtgtgtgctgatcccccgtccccctgtggagatggaggcgtgtgctgatcccccatccccctgtggagatgaaggcgtgtgctgatccgccatcccactgtggagatgaaggcgtgtgctgatcccccatccccctgtggagataaaggtgtgtgctgatcccccatccccctgtggagatgaaggcgtgtgctgatccgccatcccactgtggagatgaaggcgtgtgctgatcccccatccccctgtggagatgaaggcgtgtgctcatcccccatccccctgtggagatgaaggcgtgtgctgatccgccatcccactgtggagatgaaggtgtgtgctgatcccccatccccctgtggagatgaaggtgtgtgctgatccccggtccccctgtggagatgaaggtgtgtgctgatcccccgtcctcctgtggagatgaaggcgtgtgctgatcccccgtccccctgtggagatgaaggcgtgtgctgatccgccgtccccctgtggagatgaaggcgtgtgctgatccgccatccccctgtggagatgaaggcgtgtgctcatcccccatccccctgtggagatgaaggcgtgtgctgatccgccatcccactgtggagatgaaggtgtgtgctgatcccccatccccctgtggagatgaaggcgtgtgctgatccccggtccccctgtggagatgaaggtgtgtgttgatcccccatccccctgtggaggtgaaggtgtgtgctgattactaatccacatgggcaagaacaggatgtccgtcattaccctgaatcaatgctaacacagtgccacttgagttcacattaagttagaaattgagaatctaaaggtacctttattttaactaaaaaataatttatatactgtatattgattgtgacacaatttacaaagtctgaggtgtggaacagttatttaagcattagtcaaccctggtccttaagacagttctagtaaaatgggattgtatatatttgttcaactattttgaccaaaaagttcaataaattttaaatgtttaactgga

SEQ ID NO: 20 - protein, 273 aa, Artificial Sequence
  Description of Artificial Sequence Synthetic polypeptide
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEKSGYSGAGSYQLTFGKGTKLSVIPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 21 - protein, 299 aa, Artificial Sequence
  Description of Artificial Sequence Synthetic polypeptide
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVQDSEAFFGQGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 22 - protein, 270 aa, Artificial Sequence
  Description of Artificial Sequence Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPQTGGSYIPTFGRGTSLIVHPYIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 23 - protein, 305 aa, Artificial Sequence
  Description of Artificial Sequence Synthetic polypeptide
MAPRLLCCAALSLLWAGPVNAGVTQTPKFQVLKTGQSMTLQCAQDMNHEYMSWYRQDPGMGLRLIHYSVAEGITDQGEVPNGYNVSRSTTEDFPLRLLSAAPSQTSVYFCASSYVGAAGELFFGEGSRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 24 - protein, 270 aa, Artificial Sequence; identical to SEQ ID NO: 22
  Description of Artificial Sequence Synthetic polypeptide

SEQ ID NO: 25 - protein, 305 aa, Artificial Sequence
  Description of Artificial Sequence Synthetic polypeptide
MAPRLLCCAALSLLWAGPVNAGVTQTPKFQVLKTGQSMTLQCAQDMNHEYMSWYRQDPGMGLRLIHYSVAEGITDQGEVPNGYNVSRSTTEDFPLRLLSAAPSQTSVYFCASSYVGLAGELFFGEGSRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 26 - protein, 9 aa, Human betaherpesvirus 5
NLVPMVATV

SEQ ID NO: 27 - protein, 134 aa, Homo sapiens
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPQGSTLGRLYFGRGTQLTVWPD

SEQ ID NO: 28 - protein, 142 aa, Homo sapiens
MLSPDLPDSAWNTRLLCRVMLCLLGAGSVAAGVIQSPRHLIKEKRETATLKCYPIPRHDTVYWYQQGPGQDPQFLISFYEKMQSDKGSIPDRFSAQQFSGYHSELNMSSLELGDSALYFCASSLENNQPQHFGDGTRLSILE

SEQ ID NO: 29 - protein, 133 aa, Homo sapiens
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCARNTGNQFYFGTGTSLTVIPN

SEQ ID NO: 30 - protein, 135 aa, Homo sapiens
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSPITGTGDYGYTFGSGTRLTVVE

SEQ ID NO: 31 - protein, 9 aa, Human betaherpesvirus 5
VLEETSVML

SEQ ID NO: 32 - protein, 132 aa, Homo sapiens
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAGHIYGGSQGNLIFGKGTKLSVKPN

SEQ ID NO: 33 - protein, 134 aa, Homo sapiens
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSHHQGPLETQYFGPGTRLLVLE

SEQ ID NO: 34 - protein, 20 aa, Homo sapiens
WITQCFLPVFLAQPPSGQRR

SEQ ID NO: 35 - protein, 20 aa, Homo sapiens
LSLLMWITQCFLPVFLAQPP

SEQ ID NO: 36 - protein, 9 aa, Homo sapiens
LLASSILCA

SEQ ID NO: 37 - protein, 9 aa, Homo sapiens
YLLLASSIL

SEQ ID NO: 38 - protein, 9 aa, Homo sapiens
RILVNLSMV

SEQ ID NO: 39 - protein, 22 aa, Homo sapiens
SILCALIVFWKYRRFQRNTGEM

SEQ ID NO: 40 - protein, 11 aa, Homo sapiens
ILNNFPHSIAR

SEQ ID NO: 41 - DNA, 399 bp, Homo sapiens
atggagactgttctgcaagtactcctagggatattggggttccaagcagcctgggtcagtagccaagaactggagcagagtcctcagtccttgatcgtccaagagggaaagaatctcaccataaactgcacgtcatcaaagacgttatatggcttatactggtataagcaaaagtatggtgaaggtcttatcttcttgatgatgctacagaaaggtggggaagagaaaagtcatgaaaagataactgccaagttggatgagaaaaagcagcaaagttccctgcatatcacagcctcccagcccagccatgcaggcatctacctctgtggagcagacatagtagactatggtcagaattttgtctttggtcccggaaccaggttgtccgtgctgccctat

SEQ ID NO: 42 - DNA, 387 bp, Homo sapiens
atgctctgctctctccttgcccttctcctgggcactttctttggggtcagatctcagactattcatcaatggccagcgaccctggtgcagcctgtgggcagcccgctctctctggagtgcactgtggagggaacatcaaaccccaacctatactggtaccgacaggctgcaggcaggggcctccagctgctcttctactccgttggtattggccagatcagctctgaggtgccccagaatctctcagcctccagaccccaggaccggcagttcatcctgagttctaagaagctccttctcagtgactctggcttctatctctgtgcctggaggcgccggggttacgagcagtacttcgggccgggcaccaggctcacggtcacagag

SEQ ID NO: 43 - DNA, 411 bp, Homo sapiens
atgaagacatttgctggattttcgttcctgtttttgtggctgcagctggactgtatgagtagaggagaggatgtggagcagagtcttttcctgagtgtccgagagggagacagctccgttataaactgcacttacacagacagctcctccacctacttatactggtataagcaagaacctggagcaggtctccagttgctgacgtatattttttcaaatatggacatgaaacaagaccaaagactcactgttctattgaataaaaaggataaacatctgtctctgcgcattgcagacacccagactggggactcagctatctacttctgtgcagagaagagcgggtactctggggctgggagttaccaactcactttcgggaaggggaccaaactctcggtcataccaaat

SEQ ID NO: 44 - DNA, 381 bp, Homo sapiens
atgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttcaagacagtgaagctttctttggacaaggcaccagactcacagttgtagag

SEQ ID NO: 45 - DNA, 402 bp, Homo sapiens
atggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcctcagggctcaaccctggggaggctatactttggaagaggaactcagttgactgtctggcctgat

SEQ ID NO: 46 - DNA, 426 bp, Homo sapiens
atgcttagtcctgacctgcctgactctgcctggaacaccaggctcctctgccgtgtcatgctttgtctcctgggagcaggttcagtggctgctggagtcatccagtccccaagacatctgatcaaagaaaagagggaaacagccactctgaaatgctatcctatccctagacacgacactgtctactggtaccagcagggtccaggtcaggacccccagttcctcatttcgttttatgaaaagatgcagagcgataaaggaagcatccctgatcgattctcagctcaacagttcagtggctatcattctgaactgaacatgagctccttggagctgggggactcagccctgtacttctgtgccagcagcttagagaacaatcagccccagcattttggtgatgggactcgactctccatcctagag

SEQ ID NO: 47 - DNA, 399 bp, Homo sapiens
atggagaagaatcctttggcagccccattactaatcctctggtttcatcttgactgcgtgagcagcatactgaacgtggaacaaagtcctcagtcactgcatgttcaggagggagacagcaccaatttcacctgcagcttcccttccagcaatttttatgccttacactggtacagatgggaaactgcaaaaagccccgaggccttgtttgtaatgactttaaatggggatgaaaagaagaaaggacgaataagtgccactcttaataccaaggagggttacagctatttgtacatcaaaggatcccagcctgaagactcagccacatacctctgtgcccgaaacaccggtaaccagttctattttgggacagggacaagtttgacggtcattccaaat

SEQ ID NO: 48 - DNA, 405 bp, Homo sapiens
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagtcctatcaccgggacaggggactatggctacaccttcggttcggggaccaggttaaccgttgtagag

SEQ ID NO: 49 - DNA, 396 bp, Homo sapiens
atgctactcatcacatcaatgttggtcttatggatgcaattgtcacaggtgaatggacaacaggtaatgcaaattcctcagtaccagcatgtacaagaaggagaggacttcaccacgtactgcaattcctcaactactttaagcaatatacagtggtataagcaaaggcctggtggacatcccgtttttttgatacagttagtgaagagtggagaagtgaagaagcagaaaagactgacatttcagtttggagaagcaaaaaagaacagctccctgcacatcacagccacccagactacagatgtaggaacctacttctgtgcaggacacatttatggaggaagccaaggaaatctcatctttggaaaaggcactaaactctctgttaaaccaaat

SEQ ID NO: 50 - DNA, 402 bp, Homo sapiens
atgggctccaggctgctctgttgggtgctgctttgtctcctgggagcaggcccagtaaaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagccaccatcaggggccgttagagacccagtacttcgggccaggcacgcggctcctggtgctcgag

SEQ ID NO: 51 - protein, 27 aa, Artificial Sequence
  Description of Artificial Sequence Synthetic peptide
RAKRSGSGATNFSLLKQAGDVEENPGP

SEQ ID NO: 52 - protein, 6 aa, Artificial Sequence
  Description of Artificial Sequence Synthetic 6xHis tag
HHHHHH